(19) United States
(12) Patent Application Publication
(10) Pub. No.: US 2005/0042213 A1
(43) Pub. Date: Feb. 24, 2005

(54) Methods and pharmaceutical compositions for modulating heparanase activation and uses thereof

(75) Inventors: Joel M. Gelder, Jarusalem (IL); Daphna Miron, Rehovot (IL)

Correspondence Address: Martin Moynihan;c/o ANTHONY CASTORINA, SUITE 207, 2001 JEFFERSON DAVIS HIGHWAY, ARLINGTON, VA, 22202, US

(73) Assignee: InSight Biopharmaceuticals Ltd.

(21) Appl. No.: 10/916,598

(22) Filed: Aug. 12, 2004

Related U.S. Application Data
(60) Provisional application No. 60/535,492, filed on Jan. 12, 2004.
(60) Provisional application No. 60/494,800, filed on Aug. 14, 2003.

(51) Int. Cl. A61K 38/48; C12N 9/42

(52) U.S. Cl. 424/94.64

(57) ABSTRACT

Methods of identifying proteases participating in heparanase activation, methods and pharmaceutical compositions useful in modulating heparanase activation and methods of modulating biological processes depending, at least in part, on heparanase activity are disclosed.

This application claims the benefit of priority of U.S. provisional patent application No. ______, filed Aug. 14, 2003, and ______, filed Jan. 12, 2004, both are incorporated by reference herein.

### FIELD AND BACKGROUND OF THE INVENTION

The present invention relates to methods and pharmaceutical compositions for modulating heparanase activation, i.e., inhibiting or accelerating heparanase activation, and to medical uses of such methods and pharmaceutical compositions. The present invention further relates to methodologies by which to identify proteases participating in heparanase activation.

Proteoglycans (PGs):

Proteoglycans (previously named mucopolysaccharides) are remarkably complex molecules and are found in every tissue of the body. They are associated with each other and also with the other major structural components such as collagen and elastin. Some PGs interact with certain adhesive proteins, such as fibronectin and laminin. The long extended nature of the polysaccharide chains of PGs, the glycosaminoglycans (GAGs), and their ability to gel, allow relatively free diffusion of small molecules, but restrict the passage of large macromolecules. Because of their extended structures and the huge macromolecular aggregates they often form, they occupy a large volume of the extracellular matrix relative to proteins (Murry R K and Keeley F W; Biochemistry, Ch. 57. pp. 667-85).

Heparan Sulphate Proteoglycans (HSPGs):

HSPGs are acidic polysaccharide-protein conjugates associated with cell membranes and extracellular matrices. HSPGs bind avidly to a variety of biologic effector molecules, including extracellular matrix components, growth factor, growth factor binding proteins, cytokines, cell adhesion molecules, proteins of lipid metabolism, degradative enzymes, and protease inhibitors. Owing to these interactions, HSPGs play a dynamic role in biology; in fact most functions of the proteoglycans are attributable to the heparan sulphate (HS) chains, contributing to cell-cell interactions and cell growth and differentiation in a number of systems. HS maintains tissue integrity and endothelial cell function. It serves as an adhesion molecule and presents adhesion-inducing cytokines (especially chemokines), facilitating localization and activation of leukocytes. HS modulates the activation and the action of enzymes secreted by inflammatory cells. The functions of HS changes during the course of the immune response are due to changes in the metabolism of HS and to the differential expression of and competition between HS-binding molecules. (Selvan R S et al; Ann. NY Acad. Sci. 1996, 797: 127-39).

HSPGs are also prominent components of blood vessels (Wight T N et al; Arteriosclerosis, 1989, 9: 1-20). In large vessels HSPGs are concentrated mostly in the intima and inner media, whereas in capillaries HSPGs are found mainly in the subendothelial basement membrane, where they support proliferating and migrating endothelial cells and stabilize the structure of the capillary wall. The ability of HSPGs to interact with extracellular matrix (ECM) macromolecules such as collagen, lamin in and fibronectin, and with different attachment sites on plasma membranes suggests a key role for this proteoglycan in the self-assembly and insolubility of ECM components, as well as in cell adhesion and locomotion.

Heparanase—a GAGs Degrading Enzyme:

Degradation of GAGs is carried out by a battery of lysosomal hydrolases. One important enzyme involved in the catabolism of certain GAGs is heparanase. It is an endo-β-glucuronidase that cleaves heparan sulphate at specific interchain sites.

The enzymatic degradation of glycosaminoglycans is reviewed By Ernst et al. (Critical Reviews in Biochemistry and Molecular Biology, 30(5):387-444 (1995). The common feature of GAGs structure is repeated disaccharide units consisting of a uronic acid and hexosamine. Various GAGs differ in the composition of the disaccharide units and in type and level of modifications, such as C5-epimerization and N or O-sulfation. Sulphated GAGs include heparin, heparan sulphate, chondroitin sulphate, dermatan sulphate and keratan sulphate. Heparan sulphate and heparin are composed of repeated units of glucosamine and glucuronic/iduronic acid, which undergo modifications such as C5-epimerization, N-sulfation and O-sulfation. Heparin is characterized by a higher level of modifications than heparan sulphate.

GAGs can be depolymerized enzymatically either by eliminative cleavage with lyases (EC 4.2.2.-) or by hydrolytic cleavage with hydrolases (EC 3.2.1.-). Often, these enzymes are specific for residues in the polysaccharide chain with certain modifications. GAGs degrading lyases are mainly of bacterial origin. In the eliminative cleavage, C5 hydrogen of uronic acid is abstracted, forming an unsaturated C4-5 bond, whereas in the hydrolytic mechanism a proton is donated to the glycosidic oxygen and creating an O5 oxonium ion followed by water addition which neutralizes the oxonium ion and saturates all carbons (Lindhart et al. 1986, Appl. Biochem. Biotech. 12:135-75). The lyases can only cleave linkages on the non-reducing side of the of uronic acids, as the carboxylic group of uronic acid participates in the reaction. The hydrolases, on the other hand, can be specific for either of the two bonds in the repeating disaccharides. In pages 414 and 424 of the review, tables 8 and 14, Ernst et al. list the known GAG degrading enzymes. These tables describe substrate specificity, cleavage mechanism, cleavage linkage, product length and mode of action (endo/exolytic). Heparanase is defined as a GAG hydrolase which cleaves heparin and heparan sulphate at the β1,4 linkage between glucuronic acid and glucosamine. Heparanase is an endolytic enzyme and the average product length is 8-12 saccharides. The other known heparin/heparan sulphate degrading enzymes are beta-glucuronidase, alpha-L iduronidase and alpha-N acetylglucosaminidase, which are exolytic enzymes, each one cleaves a specific linkage within the polysaccharide chain and generate disaccharides. In table 8 the authors list two heparanases; platelet heparanase and tumor heparanase, which share the same substrate and mechanism of action. These two were later on found to be identical at the molecular level (Freeman et al. Biochem J. (1999) 342, 361-268, Vlodavsky et al. Nat. Med. 5(7):793-802, 1999, Hullet et al. Nature Medicine 5(7):803-809, 1999).

Heparin and heparan sulphate fragments generated via heparanase catalyzed hydrolysis are inherently characterized by saturated non-reducing ends, derivatives of N-acetyl-glucosamine. The reducing sugar of heparin or heparan sulphate fragments generated by heparanase hydrolysis contain a hydroxyl group at carbon 4 and it is therefore UV inactive at 232 nm.

Interaction of T and B lymphocytes, platelets, granulocytes, macrophages and mast cells with the subendothelial extracellular matrix (ECM) is associated with degradation of heparan sulphate by heparanase activity. The enzyme is released from intracellular compartments (e.g., lysosomes, specific granules) in response to various activation signals (e.g., thrombin, calcium ionophore, immune complexes, antigens and mitogens), suggesting its regulated involvement in inflammation and cellular immunity. (Vlodavsky I et al; Invasion Metas. 1992; 12(2): 112-27). In contrast, various tumor cells appear to express and secrete heparanase in a constitutive manner in correlation with their metastatic potential. (Nakajima M et al; J. Cell. Biochem. 1988 February; 36(2):157-67). Important processes in the tissue invasion by leukocytes include their adhesion to the luminal surface of the vascular endothelium, their passage through the vascular endothelial cell layer and the subsequent degradation of the underlying basal lamina and extracellular matrix with a battery of secreted and/or cell surface protease and glycosidase activities. Cleavage of HS by heparanase may therefore result in disassembly of the subendothelial ECM and hence may play a decisive role in extravasation of normal and malignant blood-borne cells (Vlodavsky I et al; Inv. Metast. 1992, 12: 112-27, Vlodavsky I et al; Inv. Metast. 1995, 14: 290-302).

It has been previously demonstrated that heparanase may not only function in cell migration and invasion, but may also elicit an indirect neovascular response (Vlodavsky I et al; Trends Biochem. Sci. 1991, 16: 268-71). The ECM HSPGs provide a natural storage depot for bFGF. Heparanase mediated release of active bFGF from its storage within ECM may therefore provide a novel mechanism for induction of neovascularization in normal and pathological situations (Vlodavsky I et al; Cell. Molec. Aspects. 1993, Acad. Press. Inc. pp. 327-343, Thunberg L et al; FEBS Lett. 1980, 117: 203-6). Degradation of heparan sulphate by heparanase results in the release of other heparin-binding growth factors, as well as enzymes and plasma proteins that are sequestered by heparan sulphate in basement membranes, extracellular matrices and cell surfaces. (Selvan R S et al; Ann. NY Acad. Sci. 1996, 797: 127-39).

Expression of Heparanase DNA in Animal Cells:

Stably transfected CHO cells express the human heparanase gene products in a constitutive and stable manner. Several CHO cellular clones are particularly productive in expressing heparanase, as determined by protein blot analysis and by activity assays. Although the heparanase DNA encodes for a large 543 amino acids protein (expected molecular weight about 65 kDa, SEQ ID NO: 8) the results clearly demonstrate the existence of three proteins, one of about 60 kDa (H60, SEQ ID NO: 34), another of about 45 kDa (H45, SEQ ID NO: 33) and yet another one of about 8 kDa (H8, SEQ ID NO: 35). It was found that active heparanase is a mature processed form with an apparent molecular weight of 53 kDa (H53), proteolitically cleaved from the latent heparanase precursor of about 60 kDa. This proteolytic cleavage occurs at two cleavage sites Glu¹⁰⁹-Ser¹¹⁰ (SEQ ID NO: 1) and Gln¹⁵⁷-Lys¹⁵⁸ (SEQ ID NO: 2), yielding a 8 kDa polypeptide at the N-terminus, a 45 kDa polypeptide at the C-terminus and a 6 kDa linker polypeptide (H6, SEQ ID NO: 36) that is released due to the cleavage. The formation of the heterodimer between the 8 and 45 kDa subunits is essential for heparanase enzymatic activity (M B Fairbanks et al. J. Biol. Chem. 274, 29587, 1999).

Further details pertaining to heparanase, heparanase gene and their uses can be found in, for example, PCT/US99/09256; PCT/US98/17954; PCT/US99/09255; PCT/US99/25451; PCT/IL00/00358; PCT/US99/15643; PCT/US00/03542; PCT/US99/06189; PCT/US00/03353; PCT/US00/03542; PCT/IL01/00830; PCT/IL01/00950′ PCT/IL01/00864; PCT/IL01/01169 and PCT/IL02/00362; and in U.S. Pat. Nos. 6,242,238; 5,968,822; 6,153,187; 6,177,545; and 6,190,875, the contents of all of which are hereby incorporated by reference.

Heparanase Activation:

Heparanase maturation involves the removal of the signal peptide, transforming the 65 kDa pre-pro-heparanase into a 60 kDa pro-heparanase (also referred to herein as latent heparanase or mature heparanase). The 60 kDa latent/mature heparanase is activated into an active heparanase as follows: The 60 kDa latent/mature heparanase is proteolytically cleaved twice into a 45 kDa major subunit, a 8 kDa small subunit and a 6 kDa linker that links the 45 kDa major subunit and the 8 kDa small subunit in the latent enzyme. The 45 kDa major subunit and the 8 kDa small subunit hetero-complex to form the 53 kDa active form of heparanase.

The nature of the protease(s) responsible for activating heparanase is yet unknown.

It will, nevertheless, be appreciated that by modulating the activity of these proteases one can modulate the rate of heparanase activation, hence the rate of heparanase activity and hence the rate of biological processes which depend on heparanase activity.

There is thus a widely recognized need for, and it would be highly advantageous to elucidate the mechanism of heparanase activation and to have methods and pharmaceutical compositions for modulating heparanase activation, i.e., inhibiting or increasing heparanase activation.

### SUMMARY OF THE INVENTION

According to one aspect of the present invention there is provided a method of regulating heparanase activity in a tissue, the method comprising modulating heparanase activation, thereby regulating heparanase activity in the tissue.

According to another aspect of the present invention there is provided a method of regulating a biological process depending at least in part on heparanase activity, the method comprising modulating heparanase activation, thereby regulating the biological process depending at least in part on heparanase activity.

According to yet another aspect of the present invention there is provided a method of treating a heparanase associated disease or disorder in a subject, the method comprising modulating in the subject activation of heparanase, thereby treating the heparanase associated disease or disorder in the subject.

According to still another aspect of the present invention there is provided a pharmaceutical composition for use in the treatment of heparanase-associated disease or disorder, the pharmaceutical composition comprising a therapeutically effective amount of an agent capable of modulating heparanase activation and a pharmaceutically acceptable carrier or diluent, the pharmaceutical composition is packaged in a packaging material and is identified in print in or on the packaging material for treating the heparanase-associated disease or disorder.

According to an additional aspect of the present invention there is provided method of treating a heparin binding protein-associated disease or disorder in a subject, the method comprising administering to the subject a therapeutic effective amount of an agent capable of heparin binding to the heparin binding protein, thereby treating the heparin binding protein-associated disease or disorder in the subject.

According to further features in preferred embodiments of the invention described below, the agent capable of inhibiting heparin binding to the heparin binding protein is a heparin binding agent.

According to still further features in the described preferred embodiments the heparin binding agent is a planar, positively charged compound.

According to still further features in the described preferred embodiments the planar, positively charged compound is selected from the group of compounds listed in Table 11.

According to still further features in the described preferred embodiments the agent capable of inhibiting heparin binding to the heparin binding protein is a heparin-binding protein binding agent.

According to still further features in the described preferred embodiments the heparin-binding protein binding agent is selected from the group of compounds which are listed in Table 15.

According to yet an additional aspect of the present invention there is provided a method of identifying a protease activator of heparanase, the method comprising: (a) providing a probe which comprises a mimetic of a cleavable site of heparanase and a cleavage reporting mechanism; (b) subjecting the probe to a protease; and (c) monitoring the cleavage reporting mechanism, whereby if the cleavage reporting mechanism reports of cleavage, the protease is identified as an activator of heparanase.

According to still further features in the described preferred embodiments, the method further comprising: (d) subjecting the probe to a protease in a presence of an effective amount of an inhibitor of the protease; and (e) assaying whether the cleavage reporting mechanism fails to report cleavage, whereby if the cleavage reporting mechanism fails to report cleavage, the protease is identified as an activator of heparanase.

According to still further features in the described preferred embodiments the cleavable site of heparanase is selected from the group consisting of Glu¹⁰⁹-Ser¹¹⁰ (SEQ ID NO: 1) and Gln¹⁵⁷-Lys¹⁵⁸ (SEQ ID NO: 2) in human heparanase or their equivalents in heparanases from non human, animal, origin.

According to still further features in the described preferred embodiments the mimetic is selected from the group consisting of Z-Pro-Lys-Lys-Glu-R (SEQ ID NO: 10) and Z-Glu-His-Tyr-Gln-R (SEQ ID NO: 11), whereby Z represents an optional first member of a FRET pair or an optional protecting group or Z is non existing and R represent a second member of a FRET pair or a self quenched fluorophore.

According to still further features in the described preferred embodiments the cleavage reporting mechanism comprises a quenched fluorophore.

According to still further features in the described preferred embodiments the quenched fluorophore is 7-amino-4-methylcoumarin (AMC).

According to still an additional aspect of the present invention there is provided a compound comprising Z-Pro-Lys-Lys-Glu-R or Z-Glu-His-Tyr-Gln-R, whereby Z represents an optional first member of a FRET pair or an optional protecting group or Z is non existing and R represent a second member of a FRET pair or a self quenched fluorophore.

According to a further aspect of the present invention there is provided a protease substrate mimetic comprising a peptide which comprises at least two amino acids representing a subset or all substrate residues at positions P4, P3, P2, P1, P1′, P2′, P3′, P4′ of the Glu¹⁰⁹-Ser¹¹⁰ (SEQ ID NO: 1) or the Gln¹⁵⁷-Lys¹⁵⁸ (SEQ ID NO: 2) cleavage sites of human heparanase or equivalent sites of a nonhuman heparanase, with the provision that P1 is represented, the protease substrate further comprising a cleavage reporting mechanism being covalently attached to the peptide, the cleavage reporting mechanism for reporting of cleavage of a bond immediately C terminally to P1.

According to still further features in the described preferred embodiments P4, P3, P2, P1, P1′, P2′, P3′ and P4′ are all represented.

According to still further features in the described preferred embodiments only P4, P3, P2 and P1 are represented.

According to still further features in the described preferred embodiments only P3, P2, P1, P1′, P2′, P3′ and are represented.

According to still further features in the described preferred embodiments the cleavage reporting mechanism comprises a Z group covalently attached at the N terminal of the peptide and an R group covalently attached at the C terminal of the peptide, whereby Z represents an optional first member of a FRET pair or an optional protecting group or Z is non existing and R represent a second member of a FRET pair or a self quenched fluorophore.

According to yet a further aspect of the present invention there is provided a method of producing active heparanase, the method comprsinig: (a) providing a pro-heparanase; (b) contacting the pro-heparanase with: (i) at least one protease participating in pro-heparanase activation; and (ii) heparin, heparin mimetic, heparan sulfate and/or heparan sulfate mimetic, thereby producing the heparanase.

According to still further features in the described preferred embodiments step (a) is effected by purifying the pro-heparanase from cells.

According to still further features in the described preferred embodiments the at least one protease participating in pro-heparanase activation is selected from the group consisting of a serine protease, a cysteine protease and an aspartic protease.

According to still further features in the described preferred embodiments the serine protease is elastase or cathepsin G.

According to still further features in the described preferred embodiments the cysteine protease is cathepsin B.

According to still further features in the described preferred embodiments the aspartic protease is cathepsin D.

According to still a further aspect of the present invention there is provided a kit useful for treating a heparanase associated disease or disorder in a subject, the kit comprising a container including at least one protease participating in the pro heparanase activation and/or heparin, heparin mimetic, heparan sulfate and/or heparan sulfate mimetic.

According to still further features in the described preferred embodiments the kit further comprising an additional container including pro-heparanase.

According to still a further aspect of the present invention there is provided a method of inhibiting heparanase activity, comprising contacting the heparanase with a compound having the general formula: 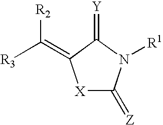
wherein:
X is O, S, NR₄ or NR₅—C(=D);
Y and Z are each independently O, S or NR₄;
R₁ is selected from the group consisting of hydrogen, a substituted or unsubstituted alkyl, a substituted or unsubstituted cycloalkyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
   —(CH₂)n-CH(R₆)-Q₁(OH);
   and
R₂ and R₃ are each independently selected from the group consisting of hydrogen, a substituted or unsubstituted aryl and a substituted or unsubstituted heteroaryl, at least one of R₂ and R₃ being the substituted or unsubstituted aryl or heteroaryl,
   and wherein:
D is O, S or NR₄;
R₄ and R₅ are each independently selected from the group consisting of hydrogen, a substituted or unsubstituted alkyl, a substituted or unsubstituted cycloalkyl and a substituted or unsubstituted aryl;
n is integer that equals 0-20;
R₆ is selected from the group consisting of hydrogen, alkyl and Q₂(OH); and
Q₁ and Q₂ are each inependently selected from the group consisting of C═O and S(═O)₂,
whereas each of the substituted alkyl, substituted alkenyl, substituted allyl, substituted cycloalkyl, substituted aryl, substituted heteroaryl and substituted heteroalicyclic independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido,
provided that either R₁ is the acid-containing moiety or at least one of the R₂ and R₃ comprises at least one C-carboxy group.

According to still a further aspect of the present invention there is provided a method of inhibiting heparanase activity, comprising contacting the heparanase with a compound having the general formula: 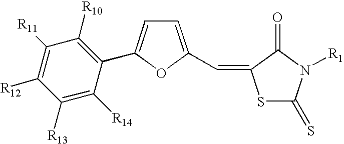
wherein:
R₁ is selected from the group consisting of hydrogen, a substituted or unsubstituted alkyl, a substituted or unsubstituted cycloalkyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
   —(CH₂)n-CH(R₆)-Q₁(OH),
whereas,
n is integer that equals 0-20;
R₆ is selected from the group consisting of hydrogen, alkyl and Q₂(OH); and
Q₁ and Q₂ are each inependently selected from the group consisting of C═O and S(═O)₂; and
R₁₀-R₁₄ are each independently selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido,
provided that either R₁ is the acid-containing moiety or at least one of the R₁₀-R₁₄ is C-carboxy.

According to still a further aspect of the present invention there is provided a method of treating a heparanase associated disease or disorder in a subject, the method comprising providing to the subject a therapeutic effective amount of a compound having the general formula: 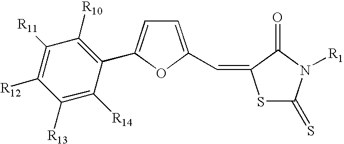
wherein:
R₁ is selected from the group consisting of hydrogen, a substituted or unsubstituted alkyl, a substituted or unsubstituted cycloalkyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
   —(CH₂)n-CH(R₆)-Q₁(OH),
whereas,
n is integer that equals 0-20;
R₆ is selected from the group consisting of hydrogen, alkyl and Q₂(OH); and
Q₁ and Q₂ are each inependently selected from the group consisting of C═O and S(═O)₂; and
R₁₀-R₁₄ are each independently selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido,
provided that either R₁ is the acid-containing moiety or at least one of the R₁₀-R₁₄ is C-carboxy.

According to still a further aspect of the present invention there is provided a method of inhibiting heparanase activation comprising contacting an inactive heparanase with an agent capable of inhibiting heparanase activation, thereby inhibiting heparanase activation.

According to still further features in the described preferred embodiments the inactive heparanase is set forth in SEQ ID NO: 34.

According to still further features in the described preferred embodiments the agent capable of inhibiting heparanase activation is selected from the group consisting of: (i) an agent capable of inhibiting at least one protease participating in the pro-heparanase activation; (ii) an agent capable of inhibiting binding of heparin to pro-heparanase; and/or (iii) an agent capable of inhibiting heparanase heterodimerization.

According to still further features in the described preferred embodiments the agent capable of inhibiting at least one protease participating in the pro-heparanase activation is selected from the group consisting of a cysteine protease inhibitor, an aspartic protease inhibitor and a serine protease inhibitor.

According to still further features in the described preferred embodiments the cysteine protease inhibitor is selected from the group consisitng of CA074, CA074Me, E-64, Cathepsin B inhibitor I (Z-Phe-Ala-CH₂F-A), Cathepsin B inhibitor II (Ac-Leu-Val-lysinal), Leupeptin, Leupeptin analogs, Cathepsin inhibitor I (Phe-Gly-NHO-Bz), Cathepsin inhibitor II (Phe-Gly-NHO-Bz-pMe), Cathepsin inhibitor III (Phe-Gly-NHO-Bz-pOme), Calpain inhibitor I (ALLN, N-Acetyl-Leu-Leu-NIe-CHO) and Calpain inhibitor II (ALLM, N-Acetyl-Leu-Leu-Met-CHO).

According to still further features in the described preferred embodiments the aspartic protease inhibitor is a cathepsin D inhibitor or a cathepsin E inhibitor each selected from the group consisting of Pepstatin A, Pepstatin A Me and a −2macroglobulin.

According to still further features in the described preferred embodiments the serine protease inhibitor is a compound having the general formula: 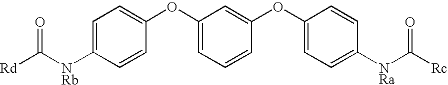
wherein:
Ra and Rb are each independently selected from the group consisting of hydrogen, alkyl, cycloalkyl or aryl; and
Rc and Rd are each independently selected from the group consisting of a substituted and unsubstituted aryl and a substituted and unsubstituted heteroaryl.

According to still further features in the described preferred embodiments each of Rc and Rd is a heteroaryl.

According to still further features in the described preferred embodiments the heteroaryl is 3-pyridine.

According to still further features in the described preferred embodiments each of Rc and Rd is a substituted aryl.

According to still further features in the described preferred embodiments the substituted aryl is a phenyl substituted by an electron withdrawing group.

According to still further features in the described preferred embodiments the agent capable of inhibiting at least one protease participating in the pro-heparanase activation is a peptide.

According to still further features in the described preferred embodiments the peptide is derived from SEQ ID NO: 36.

According to still further features in the described preferred embodiments the peptide is as set forth in SEQ ID NO: 22, 23, 24, 25, 26, 27, 28, 29 or 30.

According to still further features in the described preferred embodiments the peptide is conjugated to an electrophilic group.

According to still further features in the described preferred embodiments the electrophilic group is derived from a chemical group selected from the group consisting of aldehydes, boronates, nitriles, β-lactams, vinyl sulfones, epoxides, halomethylketones, isocoumarin and thiodiazoles.

According to still further features in the described preferred embodiments the agent capable of inhibiting binding of heparin to pro-heparanase, is a heparin-binding agent.

According to still further features in the described preferred embodiments the agent capable of inhibiting binding of heparin to pro-heparanase, is a proheparanse binding agent.

According to still further features in the described preferred embodiments the heparin binding agent is a planar, positively charged compound.

According to still further features in the described preferred embodiments the planar, positively charged compound is selected from the group of compounds listed in Table 11.

According to still further features in the described preferred embodiments the pro-heparanase binding agent is a compound having the general formula: 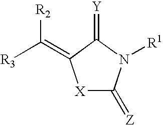
wherein:
X is O, S, NR₄ or NR₅—C(=D);
Y and Z are each independently O, S or NR₄;
R₁ is selected from the group consisting of hydrogen, a substituted or unsubstituted alkyl, a substituted or unsubstituted cycloalkyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
   —(CH₂)n-CH(R₆)-Q₁(OH);
   and
R₂ and R₃ are each independently selected from the group consisting of hydrogen, a substituted or unsubstituted aryl and a substituted or unsubstituted heteroaryl, at least one of R₂ and R₃ being the substituted or unsubstituted aryl or heteroaryl,
   and wherein:
D is O, S or NR₄;
R₄ and R₅ are each independently selected from the group consisting of hydrogen, a substituted or unsubstituted alkyl, a substituted or unsubstituted cycloalkyl and a substituted or unsubstituted aryl;
n is integer that equals 0-20;
R₆ is selected from the group consisting of hydrogen, alkyl and Q₂(OH); and

Q₁ and Q₂ are each inependently selected from the group consisting of C═O and S(═O)₂,
whereas each of the substituted alkyl, substituted alkenyl, substituted allyl, substituted cycloalkyl, substituted aryl, substituted heteroaryl and substituted heteroalicyclic independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

According to still further features in the described preferred embodiments:
X is S;
Y is O; and
Z is S or O.

According to still further features in the described preferred embodiments Z is S.

According to still further features in the described preferred embodiments,
X is NR₅—C=D;
Y is O;
Z is O or S; and
D is O or S.

According to still further features in the described preferred embodiments R₁ is the acid-containing moiety.

According to still further features in the described preferred embodiments n is greater than 1.

According to still further features in the described preferred embodiments n equals 2-5.

According to still further features in the described preferred embodiments R₁ is a substituted or unsubstituted heteroaryl.

According to still further features in the described preferred embodiments heteroaryl is selected from the group consisting of terahydrothiphenyl-1,1-dioxide and 1,5-dimethyl-2-phenyl-1,2-dihydro-3-one-pyrazolyl.

According to still further features in the described preferred embodiments R₁ is a substituted or unsubstituted aryl.

According to still further features in the described preferred embodiments the aryl is selected from the group consisting of unsubstituted phenyl, 3-halophenyl, 3-trihalomethylphenyl and 3-nitrophenyl.

According to still further features in the described preferred embodiments at least one of R₂ and R₃ is a substituted or unsubstituted heteroaryl.

According to still further features in the described preferred embodiments the heteroaryl has the general formula: 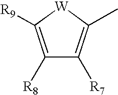
wherein:
W is O or S; and
R₇, R₈ and R₉ are each independently selected from the group consisting of hydrogen, a substituted or unsubstituted alkyl, a substituted or unsubstituted cycloalkyl, a substituted or unsubstituted aryl and a substituted or unsubstituted heteroaryl,
whereas each of the substituted alkyl, substituted cycloalkyl, substituted aryl and substituted heteroaryl independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

According to still further features in the described preferred embodiments:
R₇ and R₈ are each hydrogen; and
R₉ is an aryl having the general formula: 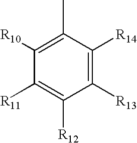
wherein each of R₁₀-R₁₄ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

According to still further features in the described preferred embodiments:
R₁₀ and R₁₄ are each independently selected from the group consisting of hydrogen, alkyl, cycloalkyl, hydroxy, alkoxy, thiohydroxy, thioalkoxy, aryloxy and thioaryloxy; and

R₁₁-R₁₃ are each independently selected from the group consisting of hydrogen, halo, nitro, trihaloalkyl and C-carboxy.

According to still further features in the described preferred embodiments:
R₇ and R₈ are each hydrogen; and
R₉ is a substituted or unsubstituted benzothiazole.

According to still further features in the described preferred embodiments at least one of R₂ and R₃ is a substituted or unsubstituted aryl having the general formula: 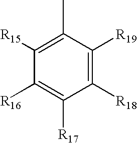
wherein each of R₁₅-R₁₉ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

According to still further features in the described preferred embodiments:
X is S;
Y is O;
Z is S; and
R₁ is the acid-containing moiety.

According to still further features in the described preferred embodiments:
n equals 2-5;
Q₁ is C═O; and
Q₂ is hydrogen.

According to still further features in the described preferred embodiments each of R₁₀-R₁₄ is indpenedently selected from the group consisting of hydrogen, nitro and halo.

According to still further features in the described preferred embodiments:
X is S;
Y is O;
Z is S; and
R₁ is selected from the group consisting of aryl, alkoxy-substituted alkyl, and heteroaryl.

According to still further features in the described preferred embodiments each of R₁₀-R₁₄ is indpenedently selected from the group consisting of hydrogen, nitro, C-carboxy and halo.

According to still further features in the described preferred embodiments at least one of R₁₀-R₁₄ is C-carboxy and the C-carboxy is a carboxylic acid group.

According to still further features in the described preferred embodiments R₁ is phenyl.

According to still further features in the described preferred embodiments R₁ is 3-methoxypropyl.

According to still further features in the described preferred embodiments R₁ is tetrahydrothiphenyl-1,1-dioxide.

According to still further features in the described preferred embodiments R₁ is 1,5-dimethyl-2-phenyl-1,2-dihydro-3-one-pyrazolyl.

According to still further features in the described preferred embodiments the agent capable of inhibiting heparanase heterodimerization is a peptide of no more than 50 amino acids.

According to still further features in the described preferred embodiments the peptide is derived from SEQ ID NO: 33 or 35.

According to still further features in the described preferred embodiments the peptide is as set forth in SEQ ID NO: 16, 17, 18, 19, 20, 21, 31 or 32.

According to still a further aspect of the present invention there is provided a method of modulating an adhesion activity of heparanase, the method comprising modulating heparin binding to heparanase, thereby modulating the adhesion activity of heparanase.

According to still further features in the described preferred embodiments modulating the adhesion activity of heparanase is decreasing adhesion activity of heparanase.

According to still further features in the described preferred embodiments modulating heparin binding to heparanase is effected by a heparin-binding agent.

According to still further features in the described preferred embodiments modulating heparin binding to heparanase is effected by an agent capable of binding a heparin binding domain of heparanase.

According to still further features in the described preferred embodiments the heparin binding agent is a planar, positively charged compound.

According to still further features in the described preferred embodiments the planar, positively charged compound is selected from the group of compounds listed in Table 11.

According to still further features in the described preferred embodiments the agent capable of binding the heparin binding domain of heparanase is a compound having the general formula: 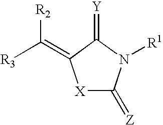
wherein:
X is O, S, NR₄ or NR₅—C(=D);
Y and Z are each independently O, S or NR₄;
R₁ is selected from the group consisting of hydrogen, a substituted or unsubstituted alkyl, a substituted or unsubstituted cycloalkyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
   —(CH₂)n-CH(R₆)-Q₁(OH);
   and
R₂ and R₃ are each independently selected from the group consisting of hydrogen, a substituted or unsubstituted aryl and a substituted or unsubstituted heteroaryl, at least one of R₂ and R₃ being the substituted or unsubstituted aryl or heteroaryl,
   and wherein:
D is O, S or NR₄;
R₄ and R₅ are each independently selected from the group consisting of hydrogen, a substituted or unsubstituted alkyl, a substituted or unsubstituted cycloalkyl and a substituted or unsubstituted aryl;
n is integer that equals 0-20;
R₆ is selected from the group consisting of hydrogen, alkyl and Q₂(OH); and
Q₁ and Q₂ are each inependently selected from the group consisting of C═O and S(═O)₂,
whereas each of the substituted alkyl, substituted alkenyl, substituted allyl, substituted cycloalkyl, substituted aryl, substituted heteroaryl and substituted heteroalicyclic independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

According to still further features in the described preferred embodiments:
X is S;
Y is O; and
Z is S or O.

According to still further features in the described preferred embodiments Z is S.

According to still further features in the described preferred embodiments:
X is NR₅—C=D;
Y is O;
Z is O or S; and
D is O or S.

According to still further features in the described preferred embodiments R₁ is the acid-containing moiety.

According to still further features in the described preferred embodiments n is greater than 1.

According to still further features in the described preferred embodiments n equals 2-5.

According to still further features in the described preferred embodiments R₁ is a substituted or unsubstituted heteroaryl.

According to still further features in the described preferred embodiments the heteroaryl is selected from the group consisting of terahydrothiphenyl-1,1-dioxide and 1,5-dimethyl-2-phenyl-1,2-dihydro-3-one-pyrazolyl.

According to still further features in the described preferred embodiments R₁ is a substituted or unsubstituted aryl.

According to still further features in the described preferred embodiments the aryl is selected from the group consisting of unsubstituted phenyl, 3-halophenyl, 3-trihalomethylphenyl and 3-nitrophenyl.

According to still further features in the described preferred embodiments at least one of R₂ and R₃ is a substituted or unsubstituted heteroaryl.

According to still further features in the described preferred embodiments the heteroaryl has the general formula: 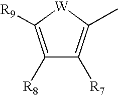
wherein:
W is O or S; and
R₇, R₈ and R₉ are each independently selected from the group consisting of hydrogen, a substituted or unsubstituted alkyl, a substituted or unsubstituted cycloalkyl, a substituted or unsubstituted aryl and a substituted or unsubstituted heteroaryl,
whereas each of the substituted alkyl, substituted cycloalkyl, substituted aryl and substituted heteroaryl independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

According to still further features in the described preferred embodiments:
R₇ and R₈ are each hydrogen; and
R₉ is an aryl having the general formula: 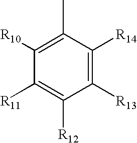
wherein each of R₁₀-R₁₄ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

According to still further features in the described preferred embodiments:
R₁₀ and R₁₄ are each independently selected from the group consisting of hydrogen, alkyl, cycloalkyl, hydroxy, alkoxy, thiohydroxy, thioalkoxy, aryloxy and thioaryloxy; and
R₁₁-R₁₃ are each independently selected from the group consisting of hydrogen, halo, nitro, trihaloalkyl and C-carboxy.

According to still further features in the described preferred embodiments:
R₇ and R₈ are each hydrogen; and
R₉ is a substituted or unsubstituted benzothiazole.

According to still further features in the described preferred embodiments at least one of R₂ and R₃ is a substituted or unsubstituted aryl having the general formula: 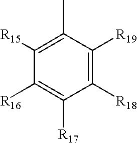
wherein each of R₁₅-R₁₉ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

According to still further features in the described preferred embodiments:
X is S;
Y is O;
Z is S; and
R₁ is the acid-containing moiety.

According to still further features in the described preferred embodiments:
n equals 2-5;
Q₁ is C═O; and
Q₂ is hydrogen.

According to still further features in the described preferred embodiments each of R₁₀-R₁₄ is indpenedently selected from the group consisting of hydrogen, nitro and halo.

According to still further features in the described preferred embodiments:
X is S;
Y is O;
Z is S; and
R₁ is selected from the group consisting of aryl, alkoxy-substituted alkyl, and heteroaryl.

According to still further features in the described preferred embodiments each of R₁₀-R₁₄ is indpenedently selected from the group consisting of hydrogen, nitro, C-carboxy and halo.

According to still further features in the described preferred embodiments at least one of R₁₀-R₁₄ is C-carboxy and the C-carboxy is a carboxylic acid group.

According to still further features in the described preferred embodiments R₁ is phenyl.

According to still further features in the described preferred embodiments R₁ is 3-methoxypropyl.

According to still further features in the described preferred embodiments R₁ is tetrahydrothiophenyl-1,1-dioxide.

According to still further features in the described preferred embodiments R₁ is 1,5-dimethyl-2-phenyl-1,2-dihydro 3-one-pyrazolyl.

The present invention successfully addresses the shortcomings of the presently known configurations by providing a new means for modulating heparanase activation, thereby modulating processes depending on heparanase activity.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in the ir entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a prior art scheme demonstrating the standard nomenclature for substrate residues and their corresponding binding sites by a protease. Reproduced from I. Schechter et al., Biochem. Biophys. Res. Comm., 27, 157, 1967.

FIG. 2 shows a plot demonstrating the kinetics of cleavage of Z-Pro-Lys-Lys-Glu-AMC (Peptide 1, SEQ ID NO: 3) by an extract from a 293 cell-line (known to be able to activate recombinantly expressed heparanase) at different pHs.

FIGS. 3a-b show Western blots demonstrating the processing of pro-heparanase in the presence of heparin (FIG. 3a) or in its absence (FIG. 3b); FIG. 3a—Lane a—Purified recombinant heparanase; Lane b—pro heparanase incubated with Cathepsin D and pepstatin; Lane 1c—pro-heparanase incubated with Cathepsin D; Lane 1d—pro-heparanase incubated with Cathepsin B and CA-074; Lane 1e—pro-heparanase incubated with cathepsin B; Lane 1f—pro-heparanase incubated with cathepsin B, cathepsin D, pepstatin and CA-074; Lane 1g—pro-heparanase incubated with cathepsin B and cathepsin D; FIG. 3b—Lane 2a—pro-heparanase incubated with cathepsin B and cathepsin D; Lane 2b—pro-heparanase incubated with cathepsin B; Lane 2c—pro-heparanase incubated with cathepsin D; Lane 2d—pro-heparanase; Lane 2e—pro-heparanase incubated with heparin; Lane 2f—purified recombinant heparanase;

FIG. 4 shows a bar graph demonstrating the activation of pro-heparanase in vitro. Pro-heparanase was incubated in the presence of 10 mg/ml Heparin sepharose (1) and with the addition of cathepsin B (2), Cathepsin D (3) Cathepsin B and Cathepsin D (4), Cathepsin B and CA-074 (5), Cathepsin D and pepstatin (6) and Cathepsin B, Cathepsin D, pepstatin and CA-074 (7). After 17 hrs of incubation, heparanase activity was determined using the DMB assay as described in U.S. Pat. No. 6,190,875.

FIG. 5 is a western blot depicting H60 processing in the presence of different proteases. Lane a—partially purified H53; Lane b—purified H60; Lane c—H60 in the presence of heparin and cathepsin B; Lane d—H60 in the presence of heparin and cathepsin D; Lane e—H60 in the presence of heparin and elastase; Lane f—H60 in the presence of heparin, cathepsin B and cathepsin D; Lane g—H60 in the presence of heparin, elastase and cathepsin B.

FIG. 6 is a bar graph depicting H60 activation in vitro. Purified H60 was incubated in the presence of heparin and various proteases as indicated in the figure.

FIG. 7 is a Western blot depicting processing of H60 in the presence of heparin or heparinomimetics. Lane A—partially purified H53; Lane B—H60; Lane C—H60 in the presence of heparin; Lane D—H60 in the presence of heparin and cathepsin D; Lane E—H60 in the presence of heparin and cathepsin D and B; Lane F—H60 in the presence of E2269 and cathepsin D and B; Lane G—H60 in the presence of heparin disaccharide IVA and cathepsin D and D; Lane H—H60.

FIG. 8 is a graph depicting the effect of heparin on pro-heparanase conformation as determined by circular dichroism.

FIG. 9 is a graph depicting the effect of the heparinomimetic compound, E2269, on heparin-dependent conformational change of heparanase, as determined by circular dichroism.

FIG. 10 is a schematic illustration of the structures of heparinomimetic molecules, which are capable of replacing heparin in pro-heparanase activation.

FIG. 11 shows a sequence alignment of the amino acid sequence of heparanases as indicated on the figure: Mouse (SEQ ID NO: 5), Bovine (SEQ ID NO: 6), Rat (SEQ ID NO: 7), Human (SEQ ID NO: 8) and Chicken (SEQ ID NO: 9).

FIG. 12 presents the formula of several aspartic proteases inhibitors which exhibit Cathepsin D selectivity, as follows: 41—sulfonamide and carboxamide derivatives; 42—modulated amyloid precursor protein and tau-protein; 43—hydroxypropylamide peptidomimetics; 44—hydroxystatine amide hydroxyphosphonate peptidomimetics; 45, 46—hydroxyamino acid amide derivatives; and 47—peptoid compounds.

FIG. 13 is a graph depicting inhibition of H60 activation by compound 63, as determined in a cell-based assay.

FIG. 14 is a western blot depicting inhibition of H60 processing in transiently transfected 293 cells in the absence of inhibitors (lane a) or presence of compound 5 (lane b), compound 63 (lane c) or compound 112 (lane d). Purified H60 is shown in lane e.

FIG. 15 is a western blot depicting H60 processing in vitro in the presence or absence of inhibitors. Lane a—purified H53; Lane b—H60 in the presence of heparin and cathepsin B; Lane c—H60 in the presence of heparin, cathepsin B and cathepsin D; Lane d—H60 in the presence of heparin, cathepsin B, cathepsin D and compound 5; Lane e—H60 in the presence of heparin, cathepsin B, cathepsin D and compound 63; Lane f—H60 in the presence of heparin, cathepsin B, cathepsin D and compound 112; Lane g—purified H60.

FIG. 16 is a bar graph depicting H60 activity in the presence or absence of inhibitors. Heparin was present in all lanes but Lane 1. Lane 1—purified H60; Lane 2—H60 in the presence of cathepsin B; Lane 3—H60 in the presence of cathepsin D; Lane 4—H60 in the presence of cathepsin B and cathepsin D; Lane 5—H60 in the presence of cathepsin B, cathepsin D and compound 63; Lane 6—H60 in the presence of cathepsin B, cathepsin D and compound 5.

FIG. 17 is a graph depicting inhibition of H60 binding to heparin by compound 5 as determined by an ELISA assay.

FIG. 18 is a western blot depicting inhibition of H60 processing by compound 128. Lane a—purified H53; Lane b—purified H60; Lane c—H60 in the presence of heparin; Lane d—H60 in the presence of heparin and cathepsin B; Lane e—H60 in the presence of heparin and cathepsin D; Lane f—H60 in the presence of heparin, cathepsin B and cathepsin D; Lane g—H60 in the presence of heparin, cathepsin B, cathepsin D and compound 128; Lane h—H60 in the presence of heparin cathepsin G and compound 128; Lane i—H60 in the presence of heparin and cathepsin G.

FIGS. 19a-c are pictures depicting three-dimensional binding models for compound 5 with each of the heparin-binding domains in human heparanase. FIG. 19a shows a binding mode of compound 5 with the heparin-binding domain KKFKNS (residues 158-163); FIG. 19b shows a binding mode of Compound 5 with the heparin-binding domain PRRKTAKM (residues 271-278); FIG. 19c shows a binding mode of compound 5 with the heparin-binding domain SKRRKLRV (residues 426-433).

FIG. 20 is a graph depicting the effect of compound 5 on tumor development in SCID mice.

### DESCRIPTION OF THE PREFERRED EMBODIMENTS

The present invention is of methods and pharmaceutical compositions for modulating heparanase activation, i.e., inhibiting or accelerating heparanase activation. The present invention is further of medical uses of such methods and pharmaceutical compositions. The present invention is still further of methodologies by which to elucidate the mechanism of heparanase activation.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Heparanase is an endo-β-glucouronidase involved in the catabolism of the glycosaminoglycans heparin and heparan sulfate. Active heparanase is a heterodimer consisting of an 8 kDa and a 45 kDa polypeptides, which is produced by a multistep-procedure involving, double proteolytic cleavage of pro-heparanase by yet unknown protease(s) and heterodimerization of active heparanase.

It is appreciated that by further elucidation of the components which participate in heparanase activation and by modulating the activity of each of these components, one can modulate the rate of heparanase activation, hence the rate of heparanase activity and hence the rate of biological processes which depend on heparanase activity.

While reducing the present invention to practice, the present inventors designed an in-vitro assay, which allows the identification of pro-heparanase activators. Using this method the present inventors identified (i) protease activators of heparanase; (ii) a critical role for heparin in pro-heparanase activation. These new targets can be efficiently utilized in the modulation of heparanase and heparanase-dependent biological processes and diseases.

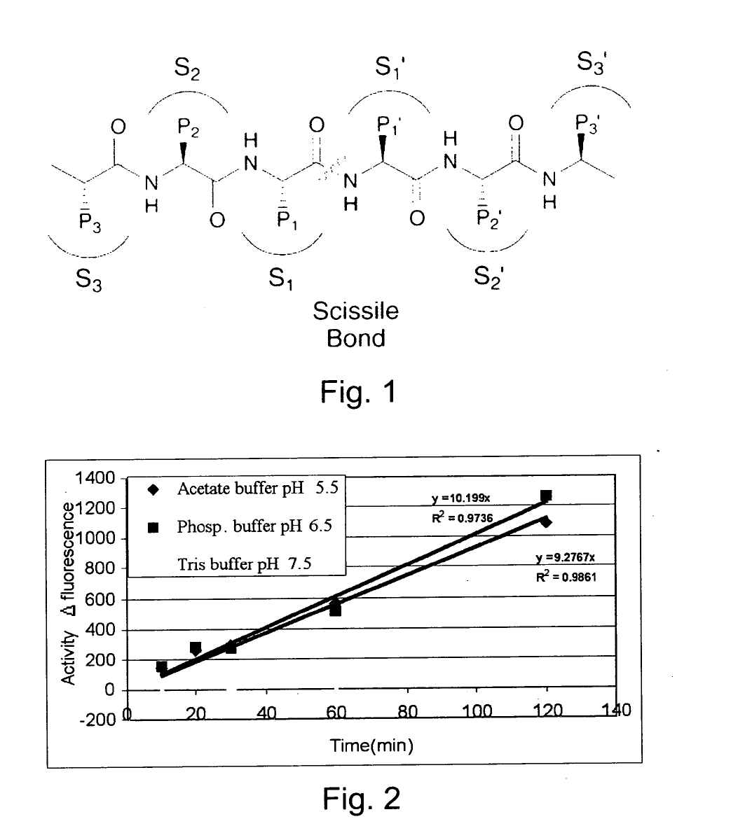

Most proteases are sequence-specific. The size and hydrophobicity/hydrophilicity of enzyme sites define possible binding amino acid side chains of polypeptide substrates. The standard nomenclature used to designate substrate/inhibitor residues (e.g., P4, P3, P2, P1, P1′, P2′, P3′, P4′) that bind to corresponding enzyme subsites (S4, S3, S2, S1, S1′, S2′, S3′, S4′) is shown in FIG. 1 (I. Schechter et al., Biochem. Biophys. Res. Comm., 27, 157, 1967). Recently it has been convincingly demonstrated for a wide range of proteases that aspartic, serine, cysteine, and metalloproteases universally bind their inhibitors/substrates in extended or β-strand conformations; that is, the peptide backbone or equivalent is drawn out in a linear arrangement (J D A Tyndall et al., J. Mol. Recognit., 12, 1, 1999).

As is shown in Example 1 of the Examples section, to investigate which protease(s) participate in heparanase activation, fluorogenic tetrapeptide substrates were synthesized based on the P4-P1 subsites of each of the cleavage sites: Glu¹⁰⁹-Ser¹¹⁰ (SEQ ID NO: 1) and the Gln¹⁵⁷-Lys¹⁵⁸ (SEQ ID NO: 2).

The peptides were labeled by the quenched fluorophore 7-amino-4-methylcoumarin (AMC), such that proteolytic cleavage of the peptide by a protease, only at the P1-AMC site, releases fluorescence. To this end, the peptides were also blocked by an N-terminal protecting group—N-carbobenzyloxy (Z), to avoid exoproteolysis.

The peptide that represents the Glu¹⁰⁹-Ser¹¹⁰ (SEQ ID NO: 1) cleavage site was Z-Pro-Lys-Lys-Glu-AMC (Peptide 1, SEQ ID NO: 3).

The peptide that represents the Gln¹⁵⁷-Lys¹⁵⁸ (SEQ ID NO: 2) cleavage site is Z-Glu-His-Tyr-Gln-AMC (Peptide 2, SEQ ID NO: 4).

Hence, according to one aspect of the present invention there is provided a method of identifying a protease activator of heparanase. The method, according to this aspect of the invention is effected by utilizing a probe which includes a mimetic of a cleavable site of heparanase and a cleavage reporting mechanism to assay the activity of a protease. In such an assay, if cleavage occurs, the reporting mechanism of the probe generates a detectable signal indicative of cleavage and the protease is identified as an activator of heparanase.

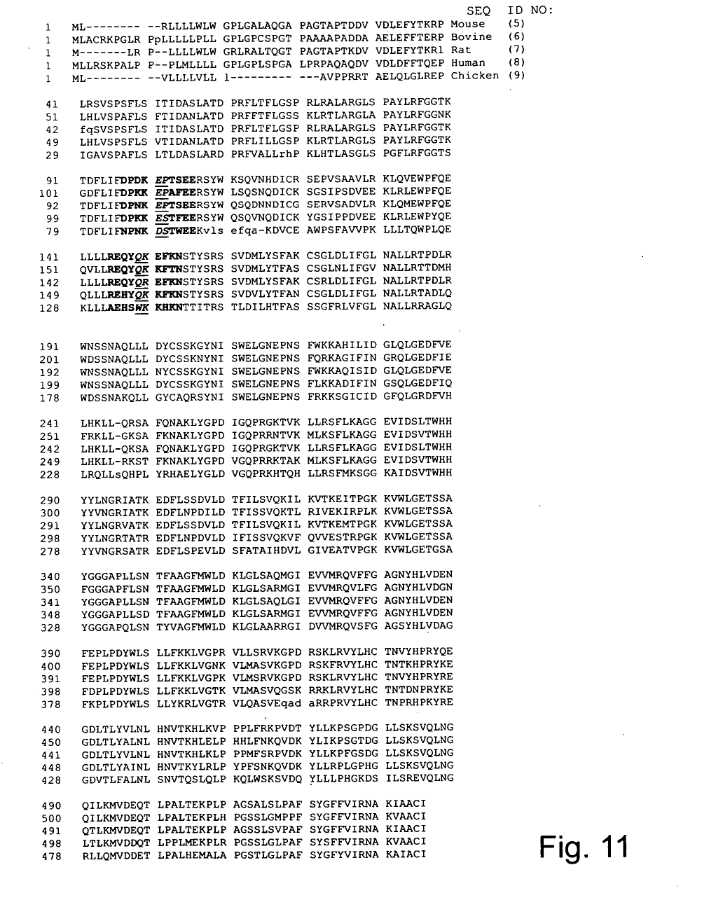

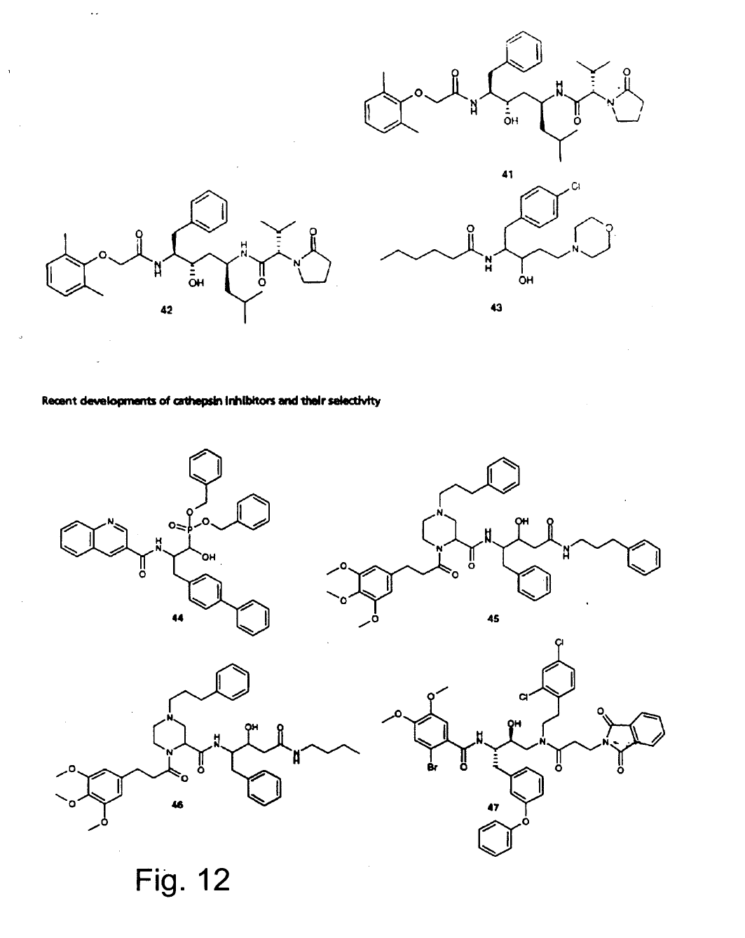

As used herein, the phrase “activator of heparanase” refers to a protease that cleaves human heparanase at the Glu¹⁰⁹-Ser¹¹⁰ (SEQ ID NO: 1) and the Gln¹⁵⁷-Lys¹⁵⁸ (SEQ ID NO: 2) cleavage sites or at the equivalent sites of orthologous heparanases. Presently, the sequences of human, rodents (mouse and rat), bovine and avian (chicken) heparanases are known (see sequence alignment of the amino acid sequence of heparanases of different species in FIG. 11). To this end see, for example, PCT/IL01/00864 and U.S. patent application Ser. No. 09/258,892.

As used herein, the phrase “mimetic of a cleavable site of heparanase” refers to a polypeptide which comprises natural and/or non-natural amino acids and that comprises or imitates a natural cleavage site of heparanase, e.g., the Glu¹⁰⁹-Ser¹¹⁰ (SEQ ID NO: 1) and the Gln¹⁵⁷-Lys¹⁵⁸ (SEQ ID NO: 2) cleavage sites of human heparanase and equivalent sites in nonhuman heparanases. Examples of mimetics of the cleavable site of human heparanase include Z-Pro-Lys-Lys-Glu-R (for the Glu¹⁰⁹-Ser¹¹⁰ (SEQ ID NO: 1) site) and Z-Glu-His-Tyr-Gln-R (for the Gln¹⁵⁷-Lys ¹⁵⁸ (SEQ ID NO: 2) site), whereby Z represents an optional first member of a FRET pair or an optional protecting group or Z is non existing and R represent a second member of a FRET pair or a self quenched fluorophore. Examples of quencher/fluorophore and fluorophore/fluorophore FRET pairs are given further below. Examples of protecting groups include N-carbobenzyloxy, t-butyloxycarbonyl and acetyl. The protecting group is designed to protect from exoproteolysis.

Preferably, the protease substrate mimetic according to this aspect of the present invention comprises a peptide which includes at least two, preferably three, four, five, six, seven or eight amino acids (either naturally occurring and/or non-natural) representing a subset or all substrate residues at positions P4, P3, P2, P1, P1′, P2′, P3′, P4′ of the Glu¹⁰⁹-Ser¹¹⁰ (SEQ ID NO: 1) or the Gln¹⁵⁷-Lys¹⁵⁸ (SEQ ID NO: 2) cleavage sites of human heparanase or equivalent sites of a non-human heparanase, with the provision that P1 is represented, the protease substrate further comprising a cleavage reporting mechanism being covalently attached to the peptide, the cleavage reporting mechanism for reporting of cleavage of a bond immediately C terminally to P1. In one option, P4, P3, P2, P1, P1′, P2′, P3′ and P4′ are all represented. In another option, only P4, P3, P2 and P1 are represented. In yet another option, only P3, P2, P1, P1′, P2′ and P3′ and are represented. Other options are also conceived.

Preferably, the cleavage reporting mechanism comprises a Z group covalently attached at the N terminal of the peptide and an R group covalently attached at the C terminal of the peptide, whereby Z represents an optional first member of a FRET pair or an optional protecting group or Z is non existing and R represent a second member of a FRET pair or a self quenched fluorophore.

A polypeptide (also referred to herein as a peptide), which forms a part of a probe used in the method of the present invention can be a naturally occurring polypeptide, comprised solely of natural amino acid residues or synthetically prepared polypeptides, comprised of a mixture of natural and modified (non-natural) amino acid residues. The nature of the polypeptide can thus be pre-determined in accordance with desired needs.

The phrase “natural amino acid” describes one of the twenty amino acids found in nature.

The phrases “modified amino acid” and “non-natural amino acid” are used herein interchangeably to describe an amino acid residue which includes a modification, e.g., at its side chain. Such modifications are well known in the art and include, for example, incorporation of a functionality group such as, but not limited to, a hydroxy group, an amino group, a carboxy group and a phosphate group within the side chain, as this phrase is defined hereinabove.

Accordingly, as used herein, the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and omithine. Furthermore, the term “amino acid” includes both D- and L-amino acids which are linked via a peptide bond or a peptide bond analog to at least one addition amino acid as this term is defined herein.

Tables 1-2 below list all the naturally occurring amino acids (Table 1) and non-conventional or modified amino acids (Table 2).

TABLE 1

| | Three-Letter | |
|---|---|---|
| Amino Acid | Abbreviation | One-letter Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional | | | |
|---|---|---|---|
| amino acid | Code | Non-conventional amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisoleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dom | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-Υ-aminobutyrate | Mgabu |
| D-α-methylalanine | Dmala | α-methylcyclohexylalanine | Mchexa |
| D-α-methylarginine | Dmarg | α-methylcy clopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-Υ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| Υ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-Υ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| Υ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamy lmethyl-glycine | Nnbhm | carbamylmethy(1)glycine | Nnbhe |
| 1-carboxy-1-(2,2-diphenyl | Nmbc | | |
| ethylamino)cyclopropane | | | |

The peptides of present invention can be biochemically synthesized such as by using standard solid phase techniques. These methods include exclusive solid phase synthesis, partial solid phase synthesis methods, fragment condensation and classical solution synthesis. These methods are preferably used when the peptide is relatively short (i.e., 10 kDa) and/or when it cannot be produced by recombinant techniques (i.e., not encoded by a nucleic acid sequence) and therefore involve different chemistry.

Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

As mentioned hereinabove, a cleavage reporting mechanism reports on the cleavage of the above-described heparanase cleavage site by the protease.

As used herein a “cleavage reporting mechanism” refers to any mechanism which would allow an observer to monitor a cleavage event. One such mechanism is the use of a quenched fluorophore, such as 7-amino-4-methylcoumarin (AMC), as a reporter, followed by and a fluoroscopy measurement Additional examples of quenched fluorophores include, MNA (4-methyoxy-2-naphtylamine); ACC (7-amino-4-carbamoylmethylcoumarin and AFC (7-amino-4 trifluoromethylcoumarin). The reporter can alternatively be radioactive and may have any one of many chemical structures, including an amino acid. Other methods can also be used such as spectroscopic methods, such as mass-spectroscopy, as well as chromatographic methods, such as thin layer chromatography (TLC) and/or high performance liquid chromatography (HPLC). All these as well as other methods can be used to monitor cleavage events.

Fluorescence resonance energy transfer (FRET) substrates allow for peptides to include entire cleavage sequences (e.g., P4-P4′) within the substrate as the fluorophore/quench or fluorophore/fluorophore pair can both be placed outside the protease recognition sequence. The fluorophore/quench arrangement is preferred as the fluorescence signal prior to cleavage is very low, and the enhancement due to proteolysis can be quite substantial.

The most commonly used quench/fluorophore pair is: DABCYL (4-(4 dimethylaminophenylazo)benzoyl)/EDANS[5-[(2-aminoethyl)amino]-naphtalene-1-sulfonic acid.

A typical FRET substrate including the entire cleavage sequence for monitoring the heparanase Gln¹⁰⁹-Ser¹¹⁰ cleavage site would be: DABCYL-Pro-Lys-Lys-Glu-Ser-Thr-Phe-Glu-EDANS (SEQ ID NO: 12).

A typical FRET substrate including the entire cleavage sequence for monitoring heparanase Gln¹⁵⁷-Lys¹⁵⁸ cleavage site would be: DABCYL-Glu-His-Tyr-Gln-Lys-Lys-Phe-Lys-EDANS (SEQ ID NO: 13)

Other common fluorophore/quench pairs used for in vitro protease assays are Abz (2-aminobenzoyl)/NP (nitrophenylalanine); Trp (tryptophan)/DNP (2,4-dinitrophenyl); Mca [(7-methoxycoumarin-4-yl)acetyl]/DNP (2,4-dinitrophenyl); Nma (N-methylanthraniloyl)/DNP (2,4-dinitrophenyl); and Abz (2-aminobenzoyl)/NY (3-nitrotyrosine).

Some fluorophore/fluorophore pairs used in certain specialized in vitro applications include salicylic acid/chelates of lanthanide ions such as Tb³⁺ and Eu⁺ and Trp (tryptophan)/Dansyl.

Thus, according to preferred embodiments of the invention, the substrates used to identify protease(s) activators of heparanase may be partial or complete fluorescence resonance energy transfer substrates, mimicking in their amino acid sequence the Gln¹⁰⁹-Ser¹¹⁰ (SEQ ID NO: 1) cleavage site and/or the Gln¹⁵⁷-Lys¹⁵⁸ (SEQ ID NO: 2) cleavage site of human heparanase or their equivalents in non-human heparanases.

According to a preferred embodiment of the invention, the method of identifying a protease activator of heparanase further comprises subjecting the probe to a protease in a presence of an effective amount of an inhibitor of the protease; and assaying whether the cleavage reporting mechanism fails to report cleavage, whereby if the cleavage reporting mechanism fails to report cleavage, the protease is identified as an activator of heparanase.

Table 3 lists some proteases categorized by their families and their known inhibitors:

TABLE 3

| Protease Family | Protease | Inhibitor |
|---|---|---|
| Aspartic Protease | Cathepsin D | Pepstatin A |
| | Cathepsin E | |
| | Pepsin | |
| | Renin | |
| Serine Protease | Trypsin | Benzamidine |
| | Thrombin | |
| | Kallikrein | |
| Serine Protease | Trypsin | AEBSF |
| | Chymotrypsin | |
| | Plasmin | |
| | Thrombin | |
| | Kallikrein | |
| Serine Protease | Trypsin | Leupeptin |
| | Thrombin | |
| | Kallikrein | |
| Cysteine Protease | Cathepsin B | Leupeptin |
| Cysteine Protease | Papain | E-64 |
| | Cathepsin B | |
| | Cathepsin L | |
| | Calpain | |
| Cysteine Protease | Cathepsin B | Calpain |
| | Cathepsin L | Inhibitor II |
| | Calpain | |
| Cysteine Protease | Cathepsin B | Cathepsin L |
| | Cathepsin L | inhibitor II |
| | Calpain | |
| Cysteine Protease | Cathepsin B | CA-074 |
| Matrix | MMP-2 | MMP-2,9 Inhibitor |
| Metalloprotease | MMP-9 | |
| Matrix | MMP-1 | Z-Pro-Leu-Gly- |
| Metalloprotease | | hydroxamate |
| Matrix | All MMP's | EDTA |
| Metalloprotease | | |
| Matrix | All MMP's | 1,10-Phenanthroline |
| Metalloprotease | | |

The method of identifying a protease activator of heparanase described hereinabove is useful in determining the participation of each of the above listed and other, non-listed, proteases in heparanase activation, and hence, also, the applicability of each of the above listed protease inhibitors in inhibiting heparanase activation.

Using the above approach, and while further reducing the present invention to practice, proteases responsible for heparanase activation were identified to be of the serine, cysteine and/or aspartic protease families, cathepsin B (a cysteine protease) and elastase and cathepsin G (both serine proteases), cathepsin D and cathepsin E (both aspartic proteases) in particular, for the Glu¹⁰⁹-Ser¹¹⁰ (SEQ ID NO: 1, cathepsin B) and Gln¹⁵⁷-Lys¹⁵⁸ (SEQ ID NO: 2) cleavage sites or other sequences in the vicinity of those cleavage sites in human heparanase.

While further reducing the present invention to practice, the present inventors uncovered that heparin plays a critical role in the activation of pro-heparanase. Two lines of evidence support involvement of heparin in heparanase activation, essentially, heparin promotes pro-heparanase cleavage in-vitro (see Example 2 of the Examples section which follows); and binding of heparin induces an extensive conformational change of pro-heparanase (see Example 3 of the Examples section which follows). It is thus suggested, that the heparin molecule wraps the pro-heparanase polypeptide by binding to the consensus sequences located at the outer surface of the protein, like in the case of other heparin-binding proteins such as annexin (Capila I. et. al., Structure 9, 57, 2001). It is conceivable that the proteolytic cleavage sites of the pro heparanase are unexposed while in their natural conformation. Binding of heparin to pro-heparanase causes a conformational change, thus exposing the protease cleavage sites to processing and allowing pro-heparanase to be activated.

Thus, the present invention contemplates the use of heparin and heparinoid mimetics for increasing pro-heparanase activation.

Although characteristic-repeating units can be attributed to heparan sulphate and heparin, these polysaccharides are characterized by great structural heterogeneity stemming from different substitution patterns, and 19 different naturally occurring uronic acid-glucosamine disaccharides have been identified so far. In addition, preparations show dispersion of molecular weight after cleavage from the proteoglycan core. Emphasizing their common features, heparin and heparan sulphate are referred to as “heparinoids” (H E Conrad, Pure Appl. Chem., 65, 787, 1993). “Heparinoids” are associated with a multitude of biological properties. These low MW heparins, heparin fractions, or other sulphated polysaccharides which are from natural source have the same, or a similar complexity as heparin and are therefore regarded as heparinoid mimetics. In the case of heparinoids, the development of mimetics is desirable to reduce enormous complexity of the polysaccharide mixtures to arrive at compounds of lower molecular weight that can be synthesized in a reasonable number of steps. A heparinoid mimetic would ideally maintain only one pharmacological activity, be structurally less complex than heparin and be easily prepared. Compounds with one defined carbohydrate backbone serving as a template for sulphates are considered as heparin mimetics. Examples include, but are not limited to, heparin saccharides; sulphated linear oligosaccharides, sulphated cyclic oligosaccharides; sulphated spaced oligosaccharides; and sulphated spaced open chain sugars (see FIG. 7 and Example 2 of the Examples section which follows). Non-carbohydrate compounds with multiple sulphate groups could also be considered as potential heparin mimetics. Examples include the sulphonated naphthalene derivative suramin and 1,3-propanediol disulphate. (Adapted from H P Wessel, Top. Curr. Chem., 187, 215, 1997).

While further reducing the present invention to practice, the present inventors have realized that use of, or interference with any one of the components involved in heparanase activation may be enough to modulate biological processes, which are governed by heparanase activity.

As is illustrated in Examples 45 of the Examples section which follows, the present inventors designed a cell-based assay, which allowed the identification of numerous inhibitors of heparanase activation. These inhibitors were functionally classified and chemically simplified to provide a comprehensive list of inhibitors, which are listed in Tables 7-13, below, each of which may be used as a potent inhibitor of heparanase activation.

Thus, according to yet another aspect of the present invention there is provided a method of regulating heparanase activity in a tissue. The method is effected by modulating heparanase activation to thereby regulate heparanase activity in the tissue.

As used herein the term “regulating heparanase activity” refers to up-regulating or down-regulating heparanase activity.

As used herein the phrase “heparanase activity” refers to any known heparanase activity (e.g., heparin or heparan sulfate cleavage activity, cell adhesion activity) or the effect of heparanase on biological processes such as cell migration, extravasation, angiogenesis, wound healing, smooth muscle cell proliferation. It will be appreciated that cell adhesion activity of heparanase is independent of its catalytic activity. Apparently, cell surface heparanase induced early stages of cell adhesion to the extracellular matrix resulting in a cascade of cell adhesion events including integrin dependent cell spreading, tyrosine phosphorylation of paxillin and reorganization of the actin cytoskeleton [Goldshmidt (2003) FASEB J. 17(9):1015-25].

As used herein the term “tissue” refers to a tissue or cell-thereof in which heparanase activity takes place. Examples include, but are not limited to, immune tissues, nervous tissues, muscle tissues, secretory tissues, kidney tissue and lung tissue.

As mentioned hereinabove, modulation of heparanase activation can be effected by modulating (i.e., increasing or inhibiting) activity or expression of any one of the components, which are involved in heparanase activation.

Thus, for example, modulation of heparanase activation may be effected by:

Modulating activity of at least one protease participating in pro-heparanase activation—As mentioned hereinabove, a double proteolytic cleavage event controls heparanase activation. Inhibition of heparanase activation may be effected using an agent which is capable of inhibiting at least one protease participating in heparanase activation (see e.g., Table 3, above). In a preferred mode of practicing the invention, inhibiting a protease participating in heparanase activation is by using compounds, typically but not always, small compounds, which may have high or low specificity to respective proteases, those with higher specificity are presently preferred.

Examples of cysteine protease inhibitors include, but are not limited to, a cathepsin B inhibitor such as CA074, CA074Me, E-64, Cathepsin B inhibitor I (Z-Phe-Ala-CH₂F-A), Cathepsin B inhibitor II (Ac-Leu-Val-lysinal), Leupeptin, Leupeptin analogs, Cathepsin inhibitor I (Phe-Gly-NHO-Bz), Cathepsin inhibitor II (Phe-Gly-NHO-Bz-pMe), Cathepsin inhibitor III (Phe-Gly-NHO-Bz-pOme), Calpain inhibitor I (ALLM, N-Acetyl-Leu-Leu-NIe-CHO) and Calpain inhibitor II (ALLM, N-Acetyl-Leu-Leu-Met-CHO).

Examples of aspartic protease inhibitors, include, but are not limited to, a cathepsin D inhibitor and a cathepsin E inhibitor such as Pepstatin A, Pepstatin A Me and a −2macroglobulin.

Examples of serine protease inhibitors, include, but are not limited to the compounds of the general formula: 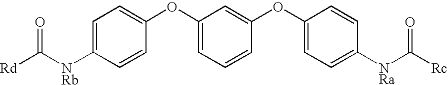
wherein:
Ra and Rb are each independently selected from the group consisting of hydrogen, alkyl, cycloalkyl or aryl; and
Rc and Rd are each independently selected from the group consisting of aryl and heteroaryl, as these terms are defined hereinbelow.

As used herein throughout, the term “alkyl” refers to a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. When substituted, the substituent group can be, for example, halo, nitro, alkoxy, aryloxy, hydroxy, thiohydroxy, thioalkoxy, thioaryloxy, alkyl, aryl, heteroaryl, heteroalicyclic, trihalomethane, C-carboxy, O-carboxy, C-amido, N-amido, S-sulfonamido and N-sulfonamido, as these terms are defined hereinbelow.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, halo, nitro, alkoxy, aryloxy, hydroxy, thiohydroxy, thioalkoxy, thioaryloxy, alkyl, aryl, heteroaryl, heteroalicyclic, trihalomethane, C-carboxy, O-carboxy, C-amido, N-amido, S-sulfonamido and N-sulfonamido, as these terms are defined hereinbelow.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, alkyl, halo, nitro, alkoxy, aryloxy, hydroxy, thiohydroxy, thioalkoxy, thioaryloxy, alkyl, aryl, heteroaryl, heteroalicyclic, trihalomethane, C-carboxy, O-carboxy, C-amido, N-amido, S-sulfonamido and N-sulfonamido, as these terms are defined hereinbelow.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, oxo, halo, nitro, alkoxy, aryloxy, hydroxy, thiohydroxy, thioalkoxy, thioaryloxy, alkyl, aryl, heteroaryl, heteroalicyclic, trihalomethane, C-carboxy, O-carboxy, C-amido, N-amido, S-sulfonamido and N-sulfonamido, as these terms are defined hereinbelow.

Preferably, Rc and Rd are the same.

As is shown in Table 13 below (see, Example 5 of the Example section that follows), preferred examples of Rc and Rd include, without limitation, a heteroaryl such as pyridinyl and furanyl, and a phenyl substituted by an electron-withdrawing group (e.g., halo). The phenyl is preferably substituted at the ortho or para positions. The pyridinyl can be a 4-pyridinyl and a 3-pyridinyl, with the first being more preferred. Other protease inhibitors which may be used in accordance with the present invention are described in Example 5, Table 12, below.

The above-described chemical inhibitors are commercially available and may be obtained from Calbiochem (EMD Biosciences, Germany), Pharmacia Corp. or Sigma (St. Louis, USA).

Alternatively, peptide agents may be used to inhibit the above-described protease, to thereby modulate heparanase activation. Such peptides may be designed and synthesized according to the protease recognition sequence on pro-heparanase (e.g., SEQ ID NOs: 1 and 2), such that these peptides act as competitive inhibitors of heparanase activation. Table 18, below lists examples of H6C-Terminus derived peptides which can be used as peptide inhibitors of heparanase activation (e.g., SEQ ID NOs: 22, 23, 24, 25, 26, 27, 28, 29 and 30). To increase affinity to nucleophilic moieties such as, thiol or hydroxyl groups, the peptides of this aspect of the present invention are preferably conjugated to an electrophilic group. Examples of electrophilic groups which may be used in accordance with the present invention are listed in Table 19 of Example 6 of the Examples section which follows.

Genetic engineering techniques may also be used to inhibit protease activity.

Such techniques may include introduction and expression or over-expression of a transgene, e.g., a transgene encoding a suitable protease (i.e., when modulating the protease activity refers to increasing protease activity) or protease inhibitor, gene knock out, whereby the gene encoding for a protease is destroyed, antisense inhibition, whereby antisense molecules are used to inhibit gene expression, siRNA inhibition, whereby small interfering RNA molecules are used to inhibit gene expression, and the like, as is further detailed below.

Gene therapy as used herein refers to the transfer of genetic material (e.g. DNA or RNA) of interest into a host to treat or prevent a genetic or acquired disease or condition or phenotype. The genetic material of interest encodes a product (e.g., a protein, polypeptide, peptide, functional RNA, antisense) whose production in vivo is desired. For example, the genetic material of interest can encode a protease, a protease inhibitor or a protease related nucleic acid (such as anti protease antisense, RNAi or snRNA) or an anti-protease intracellular antibody. For review see, in general, the text “Gene Therapy” (Advanced in Pharmacology 40, Academic Press, 1997).

Two basic approaches to gene therapy have evolved: (i) ex vivo and (ii) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to express the transfected genetic material in situ.

In in vivo gene therapy, target cells are not removed from the subject rather the genetic material to be transferred is introduced into the cells of the recipient organism in situ, that is within the recipient. In an alternative embodiment, if the host gene is defective, the gene is repaired in situ (Culver, 1998. (Abstract) Antisense DNA & RNA based therapeutics, February 1998, Coronado, Calif.). These genetically altered cells have been shown to express the transfected genetic material in situ.

The gene expression vehicle is capable of delivery/transfer of heterologous nucleic acid into a host cell. The expression vehicle may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression vehicle. Therefore, as used herein the expression vehicle may, as needed, not include the 5′UTR and/or 3′UTR of the actual gene to be transferred and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any nontranslated DNA sequence which works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al, Molecular Cloning: A Laboratory Manual, Cold Springs Harbor Laboratory, New York 1989, 1992), in Ausubel et al., Current Protocols in Molecular Biology, John Wiley and Sons, Baltimore, Md. 1989), Chang et al., Somatic Gene Therapy, CRC Press, Ann Arbor, Mich. 1995), Vega et al, Gene Targeting, CRC Press, Ann Arbor Mich. (995), Vectors: A Survey of Molecular Cloning Vectors and Their Uses, Butterworths, Boston Mass. 1988) and Gilboa et al. (Biotechniques 4 (6): 504-512, 1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector introducing and expressing recombination sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in may cases, to elude host defense mechanisms. Typically, viruses infect and propagate in specific cell types. The targeting specificity of viral utilizes its natural specificity of viral vectors utilizes its natural specificity to specifically target predetermined cell types and thereby introduce a recombinant gene into the infected cell. The vector to be used in the methods of the invention will depend on desired cell type to be targeted and will be known to those skilled in the art. For example, if breast cancer is to be treated then a vector specific for such epithelial cells would be used. Likewise, if diseases or pathological conditions of the hematopoietic system are to be treated, then a viral vector that is specific for blood cells and their precursors, preferably for the specific type of hematopoietic cell, would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the desired tissue site. However, local administration can provide a quicker and more effective treatment, administration can also be performed by, for example, intravenous or subcutaneous injection into the subject. Injection of the viral vectors into a spinal fluid can also be used as a mode of administration, especially in the case of neuro-degenerative diseases. Following injection, the viral vectors will circulate until they recognize host cells with appropriate target specificity for infection (for further details on administration methods see below).

A protease inhibitor may also be an anti-protease neutralizing antibody or an anti-protease neutralizing intracellular antibody. See for example, anti cysteine proteases neutralizing antibodies are described in Bremzl (2003) Exp. Cell Res. 283(2):206-14; matrix metalloproteases neutralizing antibodies are described in Romanic (1998) Stroke, 29, 1020-1030; Rosenberg (1998) Stroke 29, 2189-2195; Asahi (2000) J. Cereb. Blood Flow Metab. 20, 1681-1690; Ramos-DeSimone (1993) Hybridoma 12:349-63.

The term “antibody” as used in this invention includes intact molecules as well as functional fragments thereof, such as Fab, F(ab′)₂, and Fv that are capable of binding to macrophages. These functional antibody fragments are defined as follows: (i) Fab, the fragment which contains a monovalent antigen-binding fragment of an antibody molecule, can be produced by digestion of whole antibody with the enzyme papain to yield an intact light chain and a portion of one heavy chain; (ii) Fab′, the fragment of an antibody molecule that can be obtained by treating whole antibody with pepsin, followed by reduction, to yield an intact light chain and a portion of the heavy chain; two Fab′ fragments are obtained per antibody molecule; (iii) (Fab′)₂, the fragment of the antibody that can be obtained by treating whole antibody with the enzyme pepsin without subsequent reduction; F(ab′)₂ is a dimer of two Fab′ fragments held together by two disulfide bonds; (iv) Fv, defined as a genetically engineered fragment containing the variable region of the light chain and the variable region of the heavy chain expressed as two chains; and (v) Single chain antibody (“SCA”), a genetically engineered molecule containing the variable region of the light chain and the variable region of the heavy chain, linked by a suitable polypeptide linker as a genetically fused single chain molecule. Methods of making these fragments are known in the art. (See for example, Harlow and Lane, Antibodies: A Laboratory Manual, Cold Spring Harbor Laboratory, New York, 1988, incorporated herein by reference). As used in this invention, the term “epitope” means any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics. Antibody fragments useable in context of the present invention can be prepared by proteolytic hydrolysis of the antibody or by expression in E. coli or mammalian cells (e.g., Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a nonhuman species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

Gene expression of a desired protease may be modulated by designing complementary (e.g., antisense) oligonucleotides to the open reading frame, 5′, 3′, or other regulatory regions of the gene encoding the protease. Similarly, inhibition can be achieved using triple helix base-pairing which inhibits the binding of polymerases, transcription factors, or regulatory molecules to the gene [Gee et al. In: Huber and Carr (1994) Molecular and Immunologic Approaches, Futura Publishing, Mt. Kisco N.Y., pp. 163-177]. A complementary molecule may also be designed to block translation by preventing binding between ribosomes and mRNA encoding the protease.

For efficient in vivo inhibition of gene expression using antisense technology, the oligonucleotides employed must fulfill the following requirements (i) sufficient specificity in binding the target sequence; (ii) water solubility (iii) increased resistance to nuclease degradation; (iv) capability of penetration through the cell membrane, (v) low toxicity.

Antisense molecules are typically used as “chimeric antisense molecules” which contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target polynucleotide. Such modifications include but are not limited to the addition of lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

An additional region of the oligonucleotide may serve as a substrate for enzymes capable of cleaving RNA:DNA or RNA:RNA hybrids. An example for such an enzyme include RNase H, which is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense molecules useable in context of the present invention may be formed as composite structures of two or more oligonucleotides, modified oligonucleotides, as described above. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein fully incorporated by reference.

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage at sites such as GUA, GUU, and GUC. Once such sites are identified, an oligonucleotide with the same sequence may be evaluated for secondary structural features which would render the oligonucleotide inoperable. The suitability of candidate targets may also be evaluated by testing their hybridization with complementary oligonucleotides using ribonuclease protection assays.

Alternatively, downregulation of gene expression may be achieved using small interfering duplex oligonucleotides [i.e., small interfering RNA (siRNA)], which direct sequence specific degradation of mRNA through the previously described mechanism of RNA interference (RNAi) [Hutvagner and Zamore (2002) Curr. Opin. Genetics and Development 12:225-232].

As used herein, the phrase “duplex oligonucleotide” refers to an oligonucleotide structure or mimetics thereof, which is formed by either a single self-complementary nucleic acid strand or by at least two complementary nucleic acid strands. A “duplex oligonucleotide” can be composed of double-stranded RNA (dsRNA), a DNA-RNA hybrid, single-stranded RNA (ssRNA), isolated RNA (i.e., partially purified RNA, essentially pure RNA), synthetic RNA and recombinantly produced RNA.

Preferably, the protease specific small interfering duplex oligonucleotide is an oligoribonucleotide composed mainly of ribonucleic acids.

Instructions for generation of duplex oligonucleotides capable of mediating RNA interference are provided in www.ambion.com.

In human gene therapy, antisense nucleic acid technology has been one of the major tools of choice to inactivate genes where expression causes disease and is thus undesirable.

By forming a DNA/target mRNA heteroduplex, the DNA anti-sense molecule passively facilitates cleavage and degradation of the target mRNA component by endogenous RNAse H enzyme.

As an alternative to anti-sense molecules, catalytic nucleic acid molecules have shown promise as therapeutic agents for suppressing gene expression, and are widely discussed in the literature (Haseloff, J. & Gerlach, W. A. Nature 1988;334: 585; Breaker, R. R. and Joyce, G. Chemistry and Biology 1994; 1:223; Koizumi, M., et al. Nucleic Acids Research 1989;17:7059; Otsuka, E. and Koizumi, M., Japanese Patent No. 4,235,919; Kashani-Sabet, M., et al. Antisense Research and Development 1992;2:3-15; Raillard, S. A. and Joyce, G. F. Biochemistry 1996;35:11693; and Carmi, N. et al. Chemistry and Biology 1996;3:1039). Unlike conventional anti-sense inhibition, a catalytic nucleic acid molecule functions by binding to and actually cleaving its target mRNA. Cleavage of the target sequence depends on complementation of the target with the hybridizing regions of the catalytic nucleic acid, and the presence of a specific cleavage sequence. Catalytic RNA molecules (“ribozymes”) are well documented (Haseloff, J. & Gerlach, W. A. Nature 1988;334: 585; Symonds, R. H. Ann. Rev. Biochem. 1992; 61:641; and Sun, L. Q., et al. Mol. Biotechnology, 1997; 7:241), and have been shown to be capable of cleaving both RNA (Haseloff, J. & Gerlach, W. A. Nature 1988;334: 585) and DNA (Raillard, S. A. and Joyce, G. F. Biochemistry 1996;35:11693) target molecules. As described above, novel ribozymes can be designed to cleave known substrate, using either random variants of a known ribozyme or random-sequence RNA as a starting point (Pan, T. and Uhlenbeck, O. C. Biochemistry 1996;31:3887; Tsang, J. and Joyce, G. F. Biochemistry 1994;33:5966; Breaker, R. R. and Joyce, G. Chemistry and Biology 1994; 1:223). However, ribozymes may be susceptible to hydrolysis within the cells, sometimes limiting their pharmaceutical applications.

Recently, a new class of catalytic molecules called “DNAzymes” was created (Breaker, R. R. and Joyce, G. Chemistry and Biology 1995;2:655; Santoro, S. W. & Joyce, G. F. Proc. Natl, Acad. Sci. USA 1997;943:4262). DNAzymes are single-stranded, and cleave both RNA. A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M Curr Opin Mol Ther 2002;4:119-21).

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 2002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

The term enzymatic nucleic acid is used interchangeably with phrases such as ribozymes, catalytic RNA, enzymatic RNA, catalytic DNA, nucleozyme, DNAzyme, RNA enzyme, endoribonuclease, minizyme, leadzyme, oligozyme or DNA enzyme, as used in the art. All of these terminologies describe nucleic acid molecules with enzymatic activity.

Increasing heparanse activity by modulating at least one of the above-described proteases may be effected by up-regulation of the at-least one of the described proteases. This may be effected by the above described gene therapy methods or by provision of a synthetic or recombinant protease polypeptide using methods described hereinabove.

Modulating heparin binding to pro-heparanase—As is described hereinabove, the present inventors have uncovered a critical role for heparin in the activation of pro heparanase (Examples 2-3 of the Examples section).

Inhibition of heparin binding to pro-heparanase may be effected by a heparin-binding agent (or heparan-sulphate binding agent).

It will be appreciated that heparin shares common structural characteristics with DNA, as it is negatively charged and its three-dimensional structure is helical. Therefore, preferred compounds that can bind to heparin have properties (e.g. positively charged and planar) similar to the well-known DNA-intercalators (J E Scott and I H Willett, Nature, 209, 985, 1966).

Hence, as is further described in Example 5 of the Examples section, which follows, according to one preferred embodiment of this aspect of the present invention, such a heparin-binding agent is a planar, positively charged compound. Representative examples of such compounds are compounds that include conjugated or non conjugated planar aromatic rings, planar heteroaromatic rings and/or unsaturated bonds (e.g., double bonds and ketones).

Examples of planar positively charged compound inhibitors of heparanase activation are listed in Table 11, below.

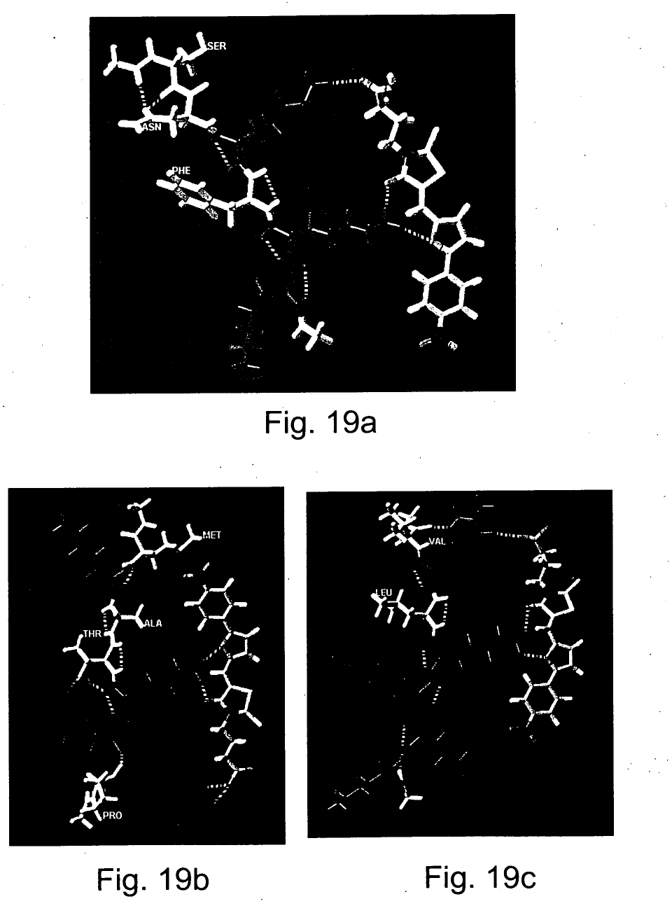

Alternatively, inhibition of heparin binding to pro-heparanase may be effected by a pro-heparanase binding agent. As used herein a pro-heparanase binding agent refers to an agent (e.g., small molecule chemical or peptide) which binds a heparin binding domain of pro-heparanase (e.g., PRRKTAKM, SKRRKLRV and QKKFKN, see FIGS. 19a-c). Such an agent is expected to bind the same domains of heparanase (H53) or similar domains of other heparin binding proteins as further described hereinbelow and to inhibit biological activity thereof (e.g., cell adhesion).

As is shown in detail in Example 5 of the Examples section which follows (see, Tables 7-10), preferred pro heparanase binding agents, according to the present invention, can be generally described by the formula: 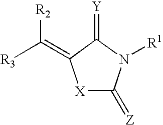
wherein:
X is O, S, NR₄ or NR₅—C(=D);
Y, Z and D are each independently O, S or NR₄;
R₁ is selected from the group consisting of hydrogen, a substituted or unsubstituted alkyl, as is defined hereinabove, a substituted or unsubstituted cycloalkyl, as is defined hereinabove, a substituted or unsubstituted alkenyl, a substituted or unsubstituted allyl, a substituted or unsubstituted aryl, as is defined hereinabove, a substituted or unsubstituted heteroaryl, as is defined hereinabove, a substituted or unsubstituted heteroalicyclic and an acid-containing moiety; and

R₂ and R₃ are each independently selected from the group consisting of hydrogen, a substituted or unsubstituted aryl and a substituted or unsubstituted heteroaryl, at least one of R₂ and R₃ being a substituted or unsubstituted aryl or heteroaryl,

and further wherein:

R₄ and R₅ are each independently selected from the group consisting of hydrogen, a substituted or unsubstituted alkyl, a substituted or unsubstituted cycloalkyl and a substituted or unsubstituted aryl.

As used herein, the phrase “an acid-containing moiety” refers to a residue of a hydrocarbon that bears a carboxylic acid and/or a sulphonic acid group. As is well accepted in the art, the term “residue” refers herein to a major portion of a molecule which is covalently linked to another molecule.

An acid-containing moiety, according to the present invention, can therefore be generally described by the general formula:
—(CH₂)n-CH(R₆)-Q₁(OH)
wherein: n is an integer that equals 0-20;
R₆ is selected from the group consisting of hydrogen, alkyl and Q₂(OH); and
Q₁ and Q₂ are each inependently selected from the group consisting of C═O and S(═O)₂.

Preferably, n is greater than 1 and, more preferably, n equals 2-5.

Further preferably, R₆ is hydrogen or alkyl, whereby a preferred alkyl is isopropyl.

The terms alkyl, cycloalkyl, aryl and heteroaryl, as used herein, are defined hereinabove.

The term “alkenyl”, as used herein, describes an alkyl group, as defined hereinabove, which consists of at least two carbon atoms and at least one carbon-carbon double bond (an unsaturated bond).

The term “allyl” describes a moiety which consists a methylene group (a —CH₂— group), covalently attached to the carbon-carbon double bond of an alkenyl group, as defined hereinabove.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido, as these terms are defined hereinbelow.

The term “halo” describes fluorine, chlorine, bromine or iodine.

The term “nitro” describes a —NO₂ group.

The term “hydroxy” describes to an —OH group.

The term “alkoxy” describes both an —O-alkyl and an —O-cycloalkyl group, as defined herein.

The term “aryloxy” describes both an —O-aryl and an —O-heteroaryl group, as defined herein.

The term “thiohydroxy” describes a —SH group.

The term “thioalkoxy” describes both a —S-alkyl group, and an —S-cycloalkyl group, as defined herein.

The term “thioaryloxy” describes both a —S-aryl and an —S-heteroaryl group, as defined herein.

The term “C-carboxy” describes a —C(═O)—O—R′ groups, where R′ is hydrogen, alkyl, cycloalkyl or aryl.

A “carboxylic acid” group refers to a C-carboxy group in which R′ is hydrogen.

The term “O-carboxy” describes a R′C(═O)O— group, where R′ is as defined herein.

The term “oxo” describes an ═O group.

The term “trihaloalkyl” describes an alkyl group in which one carbon atom is substituted by 3 halo groups, as defined herein. A preferred example is a trihalomethyl group, CX₃, where X is halo.

The term “S-sulfonamido” describes a —S(═O)₂—NR′R″ group, with R′ as defined hereinabove and R″ is as defined for for R′.

The term “N-sulfonamido” describes a R′S(═O)₂—NR″ group, where R′ and R″ are as defined herein.

The term “C-amido” describes a —C(═O)—NR′R″ group, where R′ and R″ are as defined herein.

The term “N-amido” describes a RC(═O)—NR″ group, where R′ and R″ are as defined herein.

As is further discussed in detail in Example 5 of the Examples section that follows, and is particularly demonstrated in Tables 7-10 below, preferred pro-heparanse binding agents, according to the present invention, are rhodanine derivatives having a rhodanine skeleton, such that in the general formula above X is S; Y is O; and Z is S.

However, derivatives of rhodanine analogs are also potent pro-heparanse binding agents. Representative example of rhodanine analogs include, without limitation, compounds having the general formula above, in which X is S; Y is O; and Z is O, and in which X is NR₅—C=D; Y is O; Z is O or S; and D is O or S (2-thio/oxo-dihydro-pyrimidine-4,6-dione).

It is assumed that the carbonyl moiety (Y) or the thiocarbonyl (Z), which is present in the skeleton of all such compounds, interacts, via hydrogen bonding, with one of the heparin-binding domains of pro-heparanase.

Another component which may impact the binding potency of these agents is the substituent R₁.

Preferred pro heparanase binding agents are derivatives of rhodanine or rhodanine analogs in which R₁ is an acid-containing moiety, as is defined hereinabove, or a heteroaryl such as, for example, terahydrothiophenyl-1,1-dioxide and 1,5-dimethyl-2-phenyl-1,2-dihydro-3-one-pyrazolyl. Alternatively, R₁ can be an unsubstituted phenyl, a phenyl substituted at the meta position by an electron-withrawing group (e.g., halo, trihalomethyl and nitro), or an alkyl, preferably substituted by an alkoxy group.

As is shown in the general formula above, the preferred pro-heparanse binding agents, according to the present invention, are substituted by a methylidene group, which in turn, is substituted by at least one aryl or heteroaryl (R₂ and/or R₃ in the general formula above). Preferably, R₂ (cis to the carbonyl moiety of the rhodanine skeleton) is the aryl or heteroaryl.

Preferred heteroaryls at the R₂ position can be represented by the general formula: 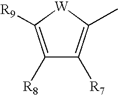
wherein:
W is O or S; and
R₇, R₈ and R₉ are each independently selected from the group consisting of hydrogen, alkyl, cycloalkyl, aryl and heteroaryl, as these terms are defined hereinabove.

Preferably, the heteroaryl is furan (W is O in the formula above), substituted at position 5 (R₉ in the formula above) by an aryl. The aryl is preferably a substituted phenyl having the general formula: 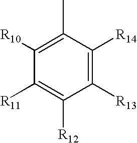
wherein each of R₁₀-R₁₄ is independently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring. Preferably, R₁₀-R₁₄ are each hydrogen, halo (e.g., chloro) and/or nitro.

As is further shown in Tables 7-10, the nature of the substituents on the phenyl ring may affect the binding potency of these agents.

Hence, the substituents at the ortho positions (R₁₀ and R₁₄) are preferably hydrogen, and/or an electron donating-group such as alkyl, cycloalkyl, hydroxy, alkoxy, thiohydroxy, thioalkoxy, aryloxy and thioaryloxy, whereby the substitutent at the meta and para positions (R₁₁-R₁₃) are preferably hydrogen and/or an electron-withdrawing group such as halo, nitro, trihaloalkyl and C-carboxy. The C-carboxy substitutent is preferably a carboxylic acid group. The presently most preferred substituents.

Alternatively, the furan- can be substituted at position 5 by a benzothiazole.

Further alternatively, the methylidene is substituted by an aryl, preferably at the cis position to the carbonyl (as R₂). The aryl is preferably substituted and can be generally presented by the general formula: 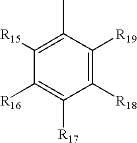
wherein each of R₁₅-R₁₉ is independently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

As is further shown in Example 5 of the Examples section that follows (see, Table 15), some of the compounds described above as pro-heparanase binding agents have a dual activity, such that in addition to inhibiting pro-heparanase activation, they inhibit heparanase activity. Preferred compounds in this category are those bearing a carboxylic acid group, either as the R₁ substituent or as one of the R₁₀-R₁₄ substituents.

The pro-heparanase binding agents of the present invention may also be used to quantify pro-heparanase and as such may be used in diagnostic applications.

It will be appreciated that agents which inhibit heparin binding to heparanase, may also be used to modulate activation of other heparin binding proteins such as those listed in Table 14, below. Table 14 also lists diseases associated with disregulated activities of such proteins which may be treated using the above-described agents (for further details see Example 5 and 8 of the Examples section which follows).

As mentioned hereinabove increasing heparanase activity may be effected by upregulation of heparin, heparan sulphate or mimetics thereof.

Modulating heparanase dimerization—As mentioned hereinabove, a final step in heparanase activation involves heterodimerization of the H45 and H8 subunits (M B Fairbanks et al., J. Biol. Chem., 274, 29587, 1999). Chemical compounds, which inhibit heparanase dimerization, or destabilize pre-exisiting dimers may be uncovered using a number of biochemical assays known in the art. Examples include, but are not limited to, cross-linking, immunoprecipitation, pull-down assay, Biacore analysis and the like.

Peptide inhibitors that competitively inhibit heparanase dimerization are preferably derived from H8 or H45. To increase bioavailability, these compounds are no more than 200 amino acids, preferably of no more than 100 amino acids, more preferably no more than 50 amino acids and even more preferably no more than 20 amino acids. Examples of heterodimerization peptide inhibitors are set forth in SEQ ID NOs: 16, 17, 18, 19, 20, 21, 31 and 32 (see Tables 16 and 17).

Increasing heparanase dimerization may be effected by cross-linkers or stabilizing antibodies which specifically stabilize heterodimers of heparanase.

It will be appreciated that the inhibitory agents of the present invention act to inhibit heparanase activation and as such may be used to inhibit any activity of heparanase (H53) which requires a preceding step of pro heparanase activation.

As used herein the phrase “heparanase activation” refers to the process of converting inactive pro-heparanase (H60) to heparanase (H53), such as protease processing and dimerization.

The present inventors have successfully shown that agents of the present invention have a therapeutic effect in vivo. As is shown in Example 8 of the Examples section, which follows, compound 5 inhibited tumor growth in SCID mice efficiently.

Thus, the above-described agents either alone, or in combination, may be used for treating heparanase associated diseases or disorders in subjects in need thereof.

As used herein the term “treating” refers to preventing, curing, reversing, attenuating, alleviating, minimizing, suppressing or halting the deleterious effects of the heparanase-associated disease or dosorder.

As used herein the term “subject” refers to a vertebrate subject, preferably a mammal e.g., human which is diagnosed with one of the diseases described hereinbelow, or alternatively is predisposed to having one of the diseases described hereinbelow.

As used herein “heparanase associated disease or disorder” refers to a disease or disorder, which depends on heparanase activity (or a biological process dependent on heparanase activity) for its onset or progression. Biological processes which depend, at least in part, on heparanase activity include, but are not limited to, cell migration (see, to this end, U.S. Pat. No. 5,968,822; Goldshmidt O. et al., FASEB J. 2003 June:17(9):1015-25; Myler H A and West J L, J Biochem 2002 June:131(6):913-22), cell invasion (see, to this end, U.S. Pat. No. 5,968,822; WO02/50243; Goldshmidt O. et al., FASEB J. 2003 June:17(9):1015-25; Parish C R et al., Biochim Biophys Acta. 2001:1471(3):M99-108;), Cell adhesion (see, to this end, Goldshmidt O. et al., FASEB J. 2003 June;17(9):1015-25), cell implantation (see, to this end, WO02/32283; U.S. application Ser. No. 09/978,297), embryo inplantation (see, to this end, WO02/32283; U.S. application Ser. No. 09/978,297; Kizaki K et al., Placenta. 2003 April;24(4):424-30; Hashizume K et al., Cloning Stem Cells. 2002;4(3):197-209; Kizaki K et al., Reproduction. 2001 April;121(4):573-80), cell transplantation (see, to this end, U.S. patent application Ser. No. 09/260,037; WO00/52149; WO02/50243), cell extravasation (see, to this end, U.S. Pat. No. 5,968,822; U.S. patent application Ser. No. 09/260,037; WO00/52149), bone formation (see, to this end, U.S. patent application Ser. No. 10/163,993; WO00/52149), HS-involved metabolic disorders, such as mucopolysaccharidoses (see, to this end, U.S. Pat. No. 6,153,187), neurodegenerative disorders, including but not limited to prion diseases (see, to this end, U.S. Pat. No. 5,968,822; Shaked G M et al., J. Biol. Chem. 2001 April 27:276(17):14324-8; Irony-Tur-Sinai M et al., J Neurol Sci. 2003 Jan. 15;206(1):49-57), hair growth (see, to this end, U.S. patent application Ser. No. 09/727,479; WO02/19962), angiogenesis (see, to this end, U.S. Pat. No. 5,968,822; U.S. patent application Ser. No. 09/727,479; WO02/19962; Elkin, M. et al., FASEB J. FASEB J. 2001 July;15(9):1661-3; Goldshmidt O et al., Proc Natl Acad Sci USA. 2002 Jul. 23;99(15):10031-6), neovascularization (see, to this end, U.S. patent application Ser. No. 09/727,479; WO02/19962; Elkin, M. et al., FASEB J. FASEB J. 2001 July;15(9):1661-3; Goldshmidt O et al., Proc Natl Acad Sci USA. 2002 Jul. 23;99(15):10031-6), cancer development (see, to this end, U.S. Pat. No. 5,968,822; Friedman Y. et al., J. American Pathology 2000: 157: 1167-1175; Vlodavsky, I. & Friedman. Y., J Clinical Investigation 2001:108: 341-347; Vlodavsky et al., Nat Med. 1999 July:5(7):793-802; Hulett M D et al., Nat Med. 1999 July:5(7):803-9), metastases formation (see, to this end, U.S. Pat. No. 5,968,822; Vlodavsky et al., Nat Med. 1999 July:5(7):793-802; Hulett M D et al., Nat Med. 1999 July:5(7):803-9), wound healing (see, to this end, U.S. Pat. No. 5,968,822; U.S. patent application Ser. No. 09/727,479; WO02/19962; U.S. patent application Ser. No. 10/341,582), inflammation, autoimmune diseases and immune recognition (see, to this end, U.S. Pat. No. 5,968,822; U.S. Pat. No. 5,968,822; Irony-Tur-Sinai M et al., J Neurol Sci. 2003 Jan. 15;206(1):49-57), atherosclerosis (see, to this end, U.S. Pat. No. 5,968,822; Pillarisetti S. Trends Cardiovasc Med. 2000 February;10(2):60-5), viral infections (see, to this end, U.S. Pat. No. 5,968,822), restenosis (see, to this end, U.S. Pat. No. 5,968,822; Francis D J et al., Circ Res. 2003 May 2:92(8):e70-7), skeletal muscle calcium kinetics (see, to this end, Jenniskens G J et al., FASEB J. 2003 May:17(8):878-80), diabetic nephropathy (see, to this end, U.S. Pat. No. 5,968,822; U.S. Pat. No. 6,177,545; Katz A et al., Isr Med Assoc J. 2002 November:4(11):996-1002), epidermal differentiation and desquamation (see, to this end, Bernard D et al., J Invest Dermatol. 2001 November;117(5):1266-73). This list of biological processes is not to be regarded exhaustive, as it is believed that many more biological processes exist in which heparanase activity plays a role.

The method according to this aspect of the present invention is effected by modulating in the subject activation of heparanase, using the agents described above, to thereby treat the heparanase associated disease or disorder in the subject.

The agents of the present invention can be provided to the subject per se, or as part of a pharmaceutical composition where they are mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the compound(s) accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer a preparation in a local rather than systemic manner, for example, via administration of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer.

For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of the active ingredient effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including any of the active ingredients useful in context of the present invention, formulated in a compatible pharmaceutical carrier may also be prepared, placed in an appropriate container, and labeled for treatment of an indicated condition.

Pharmaceutical compositions of in context of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Active heparanase (H53) is a very labile enzyme. Activity loss is a consequence of exposure to basic pH conditions, temperature up-shifting and/or enzyme dilution. This trait does not allow secretion of high levels of active heparanase overexpressed in cells, and complicates the purification process and its storage. On the other hand, the pro-heparanase is stable at various extreme conditions. This molecule is secreted at high levels and purified to above 99% purity from the medium, using two consecutive cation exchange columns. This difference may be exploited to produce, purify and store the heparanase in its latent form and activate it only closely to application.

Thus, the present invention provides a novel method of producing active heparanase. The method is effected by providing a pro-heparanase and contacting the pro-heparanase with at least one protease participating in pro-heparanase activation and heparin, heparin mimetic, heparan sulfate and/or heparan sulfate mimetic, thereby producing the active heparanase.

As used herein “pro-heparanase” refers to a mammalian pro-heparanase, such as the p60 form of human pro-heparanase Genebank accession no. AF144325 and BD074428.

Pro-heparanase can be produced from cells which were genetically modified to express the recombinant enzyme (e.g., CHO cells, U.S. Pat. No. 5,968,822). Methods of generating recombinant polypeptides are described hereinabove.

Regardless of the cellular system employed, once conditioned medium which includes the pro-heparanase is collected, the enzyme is purified therefrom using biochemical methods which are well known in the art. For example, culture medium can be applied onto a cation exchange Source 15S column. Fractions can be eluted with a salt gradient (0-1M NaCl) and tested for protein profile (SDS/PAGE followed by Coomassie staining). Pro-heparanase presence is correlated with the appearance of a protein band of about 60 kDa, consistent with the expected molecular weight of pro-heparanase. Fractions eluted from the Source-S column, containing pro-heparanase are pooled, diluted to reduce conductivity to less than 3.6 mS and applied onto another cation exchange—Fractogel COO—column. Pro-heparanase may be eluted from the column using a salt gradient of 0-1M NaCl. Aliquots of each fraction are tested for protein profile. Fractions containing the pro-heparanase are pooled.

Once, pro-heparanase is available at sufficient quantity and/or purity, it is contacted (e.g., in vitro) with it's activators (i.e., a protease participating n pro-heparanase activation and heparin, heparin mimetic, heparan sulfate and/or heparan sulfate mimetic).

In order not to lose activity of the active heparanase, contacting of the pro-heparanase with its activators is preferably effected closely to using (e.g., administering) the active enzyme. Thus, for example, contacting the pro-heparanase with the activators may be effected prior to administration of the enzyme. Alternatively, both enzyme and activators can be coadministered. Yet alternatively, the activators may be administered after the administration of the enzyme. It will be appreciated though, that in this case endogenous activators may activate the enzyme at least to some extent.

The pro-heparanase and/or activators may be packaged in a therapeutic kit (such as described hereinabove), each being packed in a separate container. Thus, the activators may be packaged in one container, while the pro-heparanase may be packaged in another container, to prevent pre-mature activation of the enzyme. Such a kit allows long storage of the pro-heparanase.

It will be further appreciated that some of the agents of the present invention which inhibit heparin binding to heparanase (see above) exhibit a dual effect, essentially in addition to inhibiting pro-heparanase activation they also inhibit heparanase activity. Table 15 below, presents results of IC₅₀ determinations towards inhibition of pro-heparanase activation (H60) and heparanase activity inhibition (H53). Structural analysis of these agents, suggests that compounds with an acidic moiety (e.g., carboxylic acid, sulfonic acid) exhibit this dual activity (see compounds 1, 4, 5, 6, 42).

Thus, the present invention further provides a method of inhibiting heparanase activity. The method is effected by contacting the heparanase with any of the pro heparanase binding agents described above which contain an acidic moiety. Preferably, the compounds include a substituted rhodanine skeleton or a rhodanine analog skeleton, as described hereinabove, attached via a methylidene to a heteroaryl (preferably furan) or aryl group, whereby the heteroaryl group is preferably attached to a substituted aryl group.

The acidic moiety can preferably be either the acid containing moiety described above at position R₁ of the rhodanine or rhodanine analog skeleton, or a carboxylic acid as a C-carboxy substituent on the the aryl group which is attached directly or indirectly, via a heteroaryl, to the rhodanine or rhodanine analog skeleton.

Preferred compound according to this aspect of the present invention can therefore be described by the general formula: 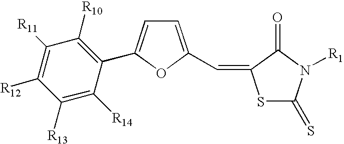
wherein:
R₁ is selected from the group consisting of hydrogen, a substituted or unsubstituted alkyl, a substituted or unsubstituted cycloakyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
   —(CH₂)n-CH(R₆)-Q₁(OH),
whereas,
n is integer that equals 0-20;
R₆ is selected from the group consisting of hydrogen, alkyl and Q₂(OH); and
Q₁ and Q₂ are each inependently selected from the group consisting of C═O and S(═O)₂; and
R₁₀-R₁₄ are each independently selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido,
whereby either R₁ is said acid-containing moiety or at least one of said R₁₀-R₁₄ is C-carboxy.

Alternatively, both R₁ and one or more of the substituents R₁₀-R₁₄ include an acidic moiety.

These compounds can be used to treat heparanase associated diseases or disorders in subjects in need thereof, as described above.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes 1-111 Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Heparanase maturation involves the removal of the signal peptide, transforming the 65 kDa pre-pro-heparanase into a 60 kDa pro-heparanase (also referred to herein as latent heparanase or mature heparanase).

The 60 kDa latent/mature heparanase is activated into an active heparanase as follows: The 60 kDa latent/mature heparanase is proteolytically cleaved twice into a 45 kDa major subunit, a 8 kDa small subunit and a 6 kDa linker that links the 45 kDa major subunit and the 8 kDa small subunit in the latent enzyme. The 45 kDa major subunit and the 8 kDa small subunit hetero-complex to form the 53 kDa active form of heparanase. The heparanase activation cleavages occur at the Glu¹⁰⁹-Ser¹¹⁰ (SEQ ID NO: 1) site and the Gln¹⁵⁷-Lys¹⁵⁸ site (SEQ ID NO: 2).

### Materials and Experimental Methods

### Example 1

### Identification of Pro-Heparanase Activating Proteases

To investigate which protease(s) participate in heparanase activation, fluorogenic peptides based on the P4-P1 pro heparanase subsites were used as reporting substrates.

Materials and Experimental Procedures

Peptides—Fluorogenic tetrapeptide substrates were synthesized based on the P4-P1 subsites of each of the cleavage sites: Glu¹⁰⁹-Ser¹¹⁰ (SEQ ID NO: 1) and the Gln¹⁵⁷-Lys¹⁵⁸ (SEQ ID NO: 2). Peptides were synthesized and labeled with the quenched fluorophore 7-amino-4-methylcoumarin by ICN Biomedicals (Irvine Calif.) such that proteolytic cleavage of the peptide by a protease, only at the P1-AMC site, releases fluorescence.

The peptide that represents the Glu¹⁰⁹-Ser¹¹⁰ (SEQ ID NO: 1) cleavage site was Z-Pro-Lys-Lys-Glu-AMC (Peptide 1, SEQ ID NO: 3).

The peptide that represents the Gln¹⁵⁷-Lys¹⁵⁸ (SEQ ID NO: 2) cleavage site is Z-Glu-His-Tyr-Gln-AMC (Peptide 2, SEQ ID NO: 4).

The peptides were also blocked by an N-terminal protecting group—N-carbobenzyloxy (Z), to avoid exoproteolysis.

Materials—Cathepsin B, cathepsin L, cathepsin D, cathepsin L inhibitor II, CA-074, CA-074Me, and MMP2,9 inhibitor were purchased from Calbiochem (San Diego USA). Leupepin, pepstatin, E64, benzamidine, Z-Pro-Leu-Gly hydroxamate, calpain inhibitor I and II and were purchased from Sigma (St. Louis, USA). Cathepsin L substrate was purchased from ICN Biomedicals (www.icnbiomed.com). Heparin sepharose CL-6B was purchased from Amersham (NJ. USA).

Cells—293 human kidney cells were a gift from Prof. Israel Vlodavsky (The Hebrew University, Jerusalem, Israel).

Proteolytic activity assay—Cathepsin B and cathepsin L activities were determined using synthetic fluorescent peptides: Z-Arg-Arg-AMC (SEQ ID NO: 14) and Z-Phe-Arg-AMC (SEQ ID NO: 15) respectively, and with the synthetic Peptide 1 (SEQ ID NO: 3), a mimetic of the heparanase cleavage site. Assay was done in a reaction mixture containing 200 mM acetate buffer, pH 5.5, 4 mM EDTA and 8 mM DTT. Following a 20 minutes incubation at 37° C., fluorescence released due to proteolytic activity was determined using a BMG POLARstar™ Galaxy fluorometer: excitation at 390 nM and emission at 460 nM.

Western blot analysis—Western blot analysis was performed using rabbit anti-heparanase polyclonal antibodies as described in U.S. Pat. Nos. 6,177,545; 6,531,129; and 6,562,950.

Activation of pro-heparanase in-vitro was performed in filter plate (Millipore, Cat. No. MADVN-65) with an assay mixture-containing heparin sepharose beads, in 200 mM acetate buffer, pH 5.5, 4 mM EDTA and 8 mM DTT, with either Cathepsin B, Cathepsin D or both. Following a 17 hours incubation at room temperature, reaction products were filtered to a new plate and quantified using di-methyl-methylene-blue, as described in U.S. Pat. No. 6,190,875.

Results

Determination of the Protease that Cleaves the Glu¹⁰⁹-Ser¹¹⁰ (SEQ ID NO: 1) Site

Cleavage of Peptide 1 by a cell extract of a human kidney 293 cell line was found to be highest at pH 5.5-6.5, while at pH 7.5 no cleavage activity was detectable (FIG. 2). Attempt to inhibit cleavage of Peptide 1 by known protease inhibitors (Table 4, below) was successful only with cysteine protease inhibitors (Leupeptin, E-64, Calpain inhibitor 1, CA-074, Cathepsin L inhibitor II), but not with any inhibitor of other protease families (pepstatin, benzamidine, MMP-2,9 inhibitor and Z-Pro-Leu-Gly hydroxamate).

TABLE 4
Inhibition of Z-Pro-Lys-Lys-Glu-AMC peptide (SEQ
ID NO: 3) cleavage by 293 cell extract, with
inhibitors of different protease families

| Inhibitor | Protease family | % Inhibition |
|---|---|---|
| Leupeptin | serine and cysteine | 97 |
| benzamidine | serine | 13 |
| pepstatin | aspartic | 0 |
| Z-Pro-Leu-Gly hydroxamate | MMPs | 0 |
| MMP 2,9 inhibitor | MMPs | 0 |
| Calpain inhibitor II | Cysteine | 98 |
| Cathepsin L inhibitor II | Cysteine | 97 |
| CA-074 | Cysteine | 96 |
| E-64 | Cysteine | 93 |

All the cysteine protease inhibitors exhibited IC₅₀ in the nanomolar range. The specific cathepsin B inhibitor CA-074 showed inhibition in the range similar to the value reported for it in the literature: IC₅₀=2.2 nM (M. Murata et al., FEBS Lett., 280, 307, 1991). It is evident from the results presented herein that cathepsin B cleaves Peptide 1, although not at the same efficiency as it cleaves its optimal substrate (described in T. Hiwasa et al., FEBS Lett., 211, 23, 1987). Cathepsin L, on the other hand, does not cleave Peptide 1 (see, Table 5).

These results are in good correlation with the substrate specificity of cathepsin B. It is well known (FCV Portaro et al., Biochem J., 347, 123, 2000) that among the mammalian cysteine proteases, cathepsin B has the unique property to cleave substrates with P2 basic residues (e.g., Arg or Lys) as in the case of heparanase.

Therefore, cathepsin B or a cathepsin B-like (homologue) protease is the protease that processes pro-heparanase at the Glu¹⁰⁹-Ser¹¹⁰ (SEQ ID NO: 1) site to yield a 8 kDa and a 51 kDa subunits.

TABLE 5
Cleavage of Peptide 1 with cathepsin B and cathepsin L

| | Cathepsin B + | Cathepsin L + | | |
|---|---|---|---|---|
| Enzyme and | Cathepsin B- | Cathepsin L- | Cathepsin B + | Cathepsin L + |
| Substrate | substrate¹ | substrate² | Peptide 1 | Peptide 1 |
| Activity | 53337 | 38254 | 8926 | 0 |
| (δfluorescence) | | | | |

¹Z-RR-AMC (calbichem, Cat. No. 219392)
²Z-FR-AMC (Enzyme systems products, Cat. No. AMC052)

Determination of the Protease that Cleaves the Gln¹⁵⁷-Lys¹⁵⁸ (SEQ ID NO: 2) Site

Peptide 2 was not cleaved by the 293 cell extract at the pH range 4.5-7.5. Although there exist some evidence that all the protease families can hydrolyze peptide substrates with amino acids from the non-prime recognition subsites, it seems that some proteases such as metalloproteases and aspartic proteases require peptides that include amino acids from both the non-prime and the prime subsites (J L Harris et al., Proc. Natl. Acad. Sci., 97, 7754, 2000).

Since serine and cysteine proteases recognize non-prime peptides, the lack of processing of this peptide may indicate that the protease that cleaves at the Gln¹⁵⁷-Lys¹⁵⁸ site belongs either to the metalloprotease or aspartic protease family.

According to the literature (H Nagase et al., Biopolymers, 40, 399, 1996), the amino acid at P1′ (Lys¹⁵⁸) of this cleavage site is not compatible for hydrolysis by metalloproteases. On the other hand, it has been shown that aspartic proteases such as cathepsin D (R E Carraway et al., Peptides, 13, 319, 1992) and cathepsin E (T. Kageyama et al., J. Biol. Chem. 270, 19135, 1995), may hydrolyze peptides containing basic amino acids at P1′.

Further analysis of the amino acids of P3-P3′ subsites of this cleavage site indicates that an aspartic protease may process the pro heparanase at the C-terminus of the 45 kDa polypeptide. The aspartic proteases most relevant to invasion and metastatic processes are cathepsin D and cathepsin E (K. Matsuo et al., Hum. Pathol., 27, 184, 1996).

### Example 2

### Heparin is Involved in Pro-Heparanase Activation as Determined by In Vitro Processing of Pro-Heparanase

The present inventors have addressed the role of heparin in pro-heparanase processing.

Materials and Experimental Procedures

Western blot analysis—Western blot analysis was performed using rabbit anti-heparanase polyclonal antibodies as described in U.S. Pat. Nos. 6,177,545; 6,531,129; and 6,562,950.

Processing and activation of pro-heparanase with cathepsin B and D—Activation of pro-heparanase in-vitro was performed in 1.5 ml tubes for western blot analysis or in a filter plate (Millipore, Cat. No. MADVN-65) for activation assay. Assay mixtures contained heparin (Sigma, H3393) or heparinomimetic molecules ethane-1,2-disulfonic acid (Sigma, E2269) and heparin disaccharide IVA (Sigma, H0895) for western blot analysis or heparin sepharose beads for activation assays, in 200 mM acetate buffer, pH 5.5, 4 mM EDTA and 8 mM DTT, with either Cathepsin B, Cathepsin D or both. Following a 17 hours of incubation at room temperature, reaction products were examined by western blot analysis or filtered to a new plate and quantified using di-methyl-methylene-blue, as described in U.S. Pat. No. 6,190,875 for activation assay.

Processing and activation of pro-heparanase with elastase—Activation of pro heparanase in-vitro was performed in 1.5 ml tubes for western blot analysis or in a filter plate (Millipore, Cat. No. MADVN-65) for activation assay. The assays were performed in assay mixtures containing heparin for western blot analysis or heparin sepharose beads for activation assays, in 100 mM Tris HCl, pH 7.5, 500 mM NaCl, with elastase from human neutrophils (Calbiochem, 324681). Following a 17 hours incubation at room temperature, reaction products were examined in western blot analysis or filtered to a new plate and quantified using di-methyl-methylene-blue, as described in U.S. Pat. No. 6,190,875 for activation assay.

Processing and activation of pro-heparanase with cathepsin G—Activation of pro-heparanase in-vitro was performed in 1.5 ml tubes for western blot analysis or in a filter plate (Millipore, Cat. No. MADVN-65) for activation assay. The assays were performed in assay mixtures containing heparin for western blot analysis or heparin sepharose beads for activation assays, in PBS, 365 mM NaCl, with 1 μ/ml cathepsin G (Sigma, C4428). Following a 17 hours incubation at room temperature, reaction products were examined in western blot analysis or filtered to a new plate and quantified using di-methyl-methylene-blue, as described in U.S. Pat. No. 6,190,875 for activation assay.

Results

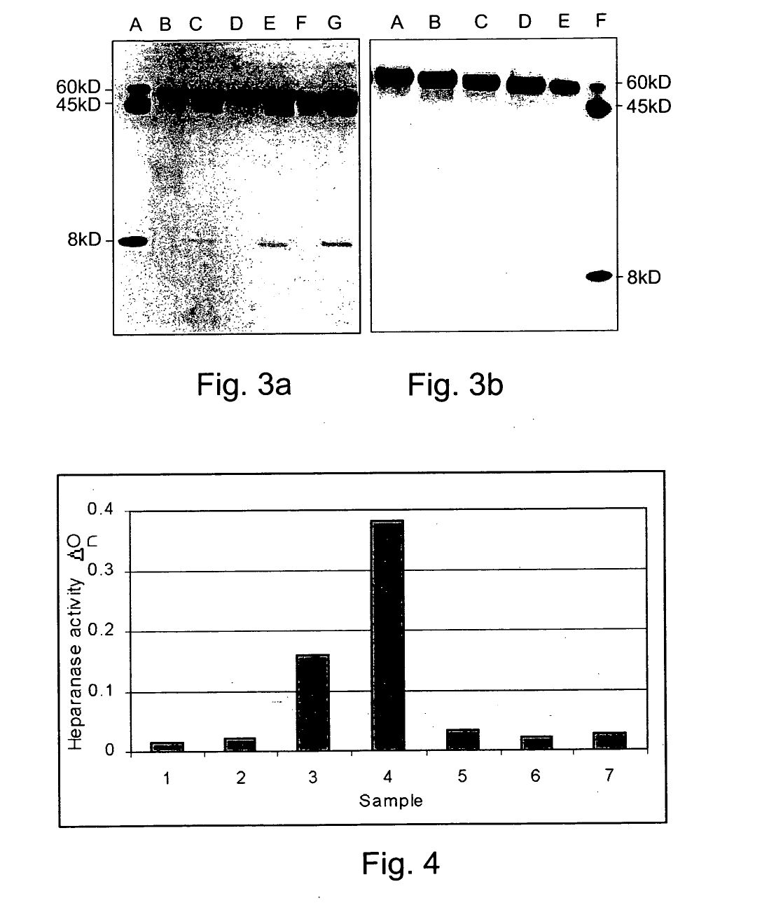

FIG. 3 shows a Western blot analysis depicting assay products and showing that in the absence of heparin, no processing occurs. When heparin was included in the reaction mixture, heparanase was processed by cathepsin D and a 45 kDa polypeptide was detected. No processing occurred when pepstatin A, an inhibitor of cathepsin D, was added to the reaction mixture. Cathepsin B in the presence of heparin but not when the cathepsin B inhibitor CA074 was added, also processed the pro-heparanase.

To examine whether cathepsin D and B in the presence of heparin may process the pro-heparanase to form the active heparanase enzyme in vitro, activity of the pro-heparanase treated with cathepsin D and/or cathepsin B when incubated with heparin-sepharose was measured (FIG. 4). Pepstatin A, an inhibitor of cathepsin D and CA-074, an inhibitor of cathepsin B, were also added as controls. Cathepsin B did not activate the pro-heparanase. Cathepsin D treatment caused an increase in heparanase activity and pepstatin A inhibited this activation. Incubation with cathepsin B and cathepsin D elevated heparanase activity to a larger extent than when incubated with cathepsin D alone. Addition of pepstatin A and CA-074 inhibited this activation.

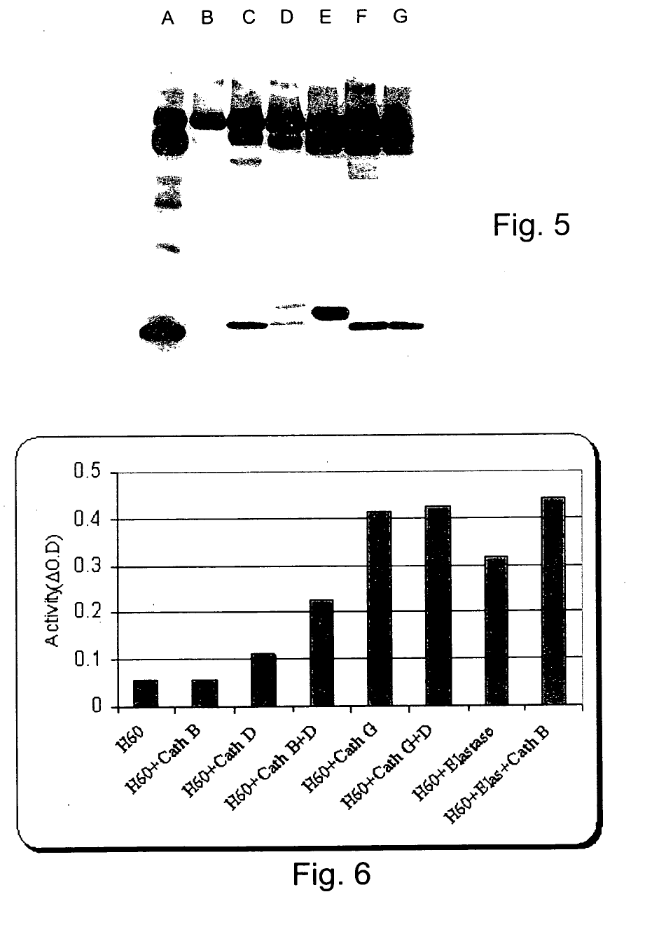

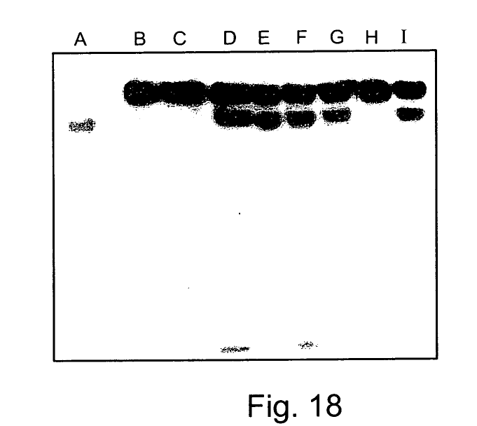

After establishing the necessity of heparin in the process of pro heparanase activation, an attempt was made to detect which protease cleaves the pro-heparanase at the N-terminus of the 45 kD polypeptide and allows generation of an active enzyme. Different proteases representing various protease families were tested for their ability to process the pro-heparanase in-vitro. Of all proteases tested (data not shown) it was found that in addition to cathepsin D, cathepsin G (FIG. 18) and elastase (FIG. 5), both serine proteases, cleave the pro-heparanase in-vitro, at or near the N-terminus of the H45. As is evident from FIG. 6, treatment of the pro heparanase with cathepsin G or elastase activated the H60. Although cathepsin B by itself did not generate an active enzyme, addition of cathepsin B but not cathepsin D to these proteases increased the activity.

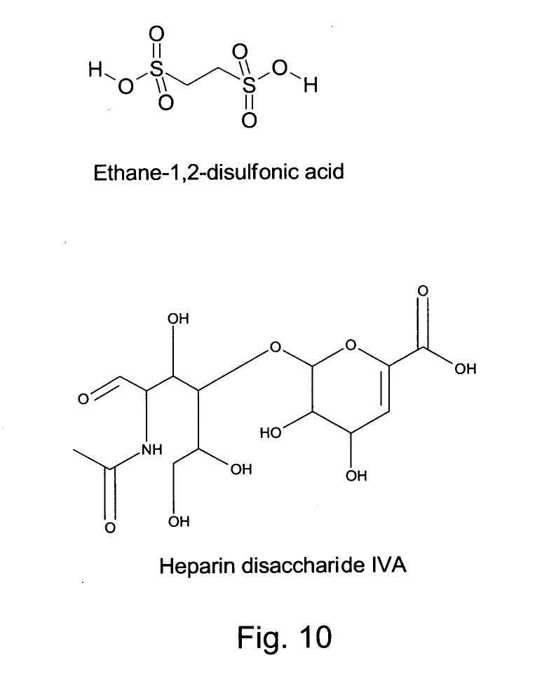

The possibility to replace heparin, in the assay mixtures for pro-heparanase activation with different heparinomimetic molecules was examined. As is evident from FIG. 7, two heparinomimetic molecules; ethane-1,2-disulfonic acid (E-2269) and heparin disaccharide IVA, allowed processing of the H60 in a similar manner to that of heparin though to a smaller extent. The structure of these molecules is presented in FIG. 10.

Altogether Examples 1 and 2 show that cathepsin B cleaves pro-heparanase at the Glu¹⁰⁹-Ser¹¹⁰ (SEQ ID NO: 1) cleavage site but does not generate an active enzyme. Furthermore, cathepsin D, cathepsin G and elastase hydrolyze the pro-heparanase in-vitro, at or near the Gln¹⁵⁷-Lys¹⁵⁸ (SEQ ID NO: 2) cleavage site. This cleavage is sufficient for activation of the enzyme, but for maximal activity processing at both cleavage sites is necessary. The results shown herein also demonstrate the importance of heparin or heparinomimetic molecules, or when in-vivo HSPGs in the activation of heparanase.

### Example 3

### Heparin Binding Induces a Conformational Change in Pro-Heparanase

Materials and Experimental Procedures

Circular Dichroism spectrometry—Circular Dichroism spectra were recorded using a JASCO 500 spetropolarimeter. Far Ultraviolet measurements (260-200 nm) were performed in a 0.1 mm demountable cuvette at 25° C., scanning at a rate of 10 nm/min. Pro-heparanase was assayed at a concentration of 0.72 mg/ml in 20 mM sodium phosphate buffer pH 6.8 supplemented with 300 mM NaCl. Effect of heparin on pro-heparanase spectra was determined at a concentration of 100 μM, and the inhibitor was added at a concentration of 15 μM. Base-line recordings were performed in the presence and absence of heparin to correct the pro-heparanase spectra.

Results

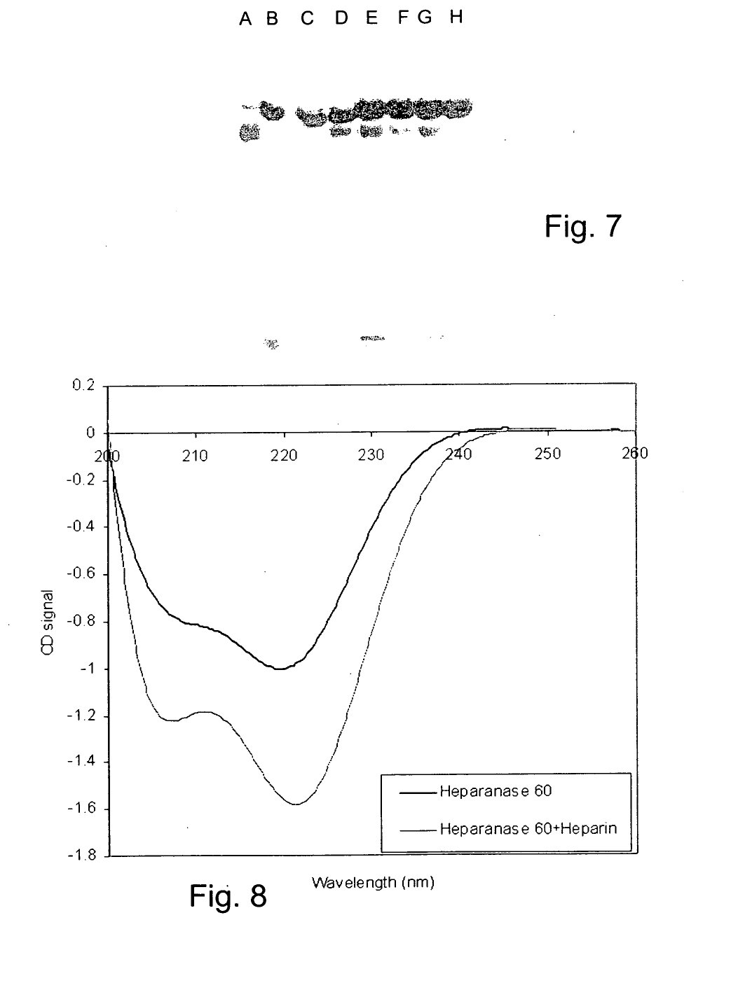

As is shown in FIG. 8, in the presence of heparin a significant conformational change in pro-heparanase was evident. These changes in conformation are summarized in Table 6, below.

TABLE 6

| | H60 − heparin | H60 + heparin |
|---|---|---|
| a helix | 0.29 | 0.44 |
| β strand | 0.26 | 0.26 |
| β turn | 0.12 | 0 |
| Un-ordered | 0.33 | 0.3 |

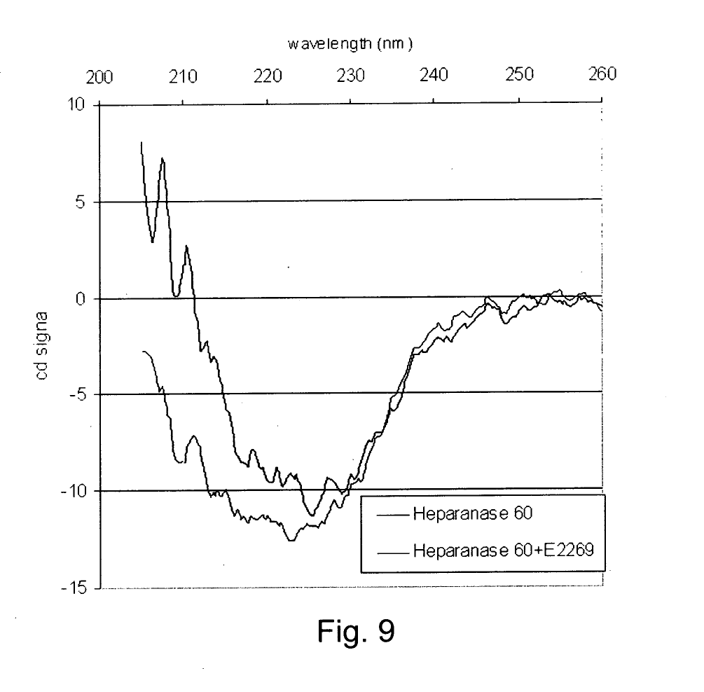

Similar changes, though to a smaller extent were also observed in the presence of E2269, indicating that heparinoid mimetic molecules function similarly (see FIG. 9).

Altogether these results suggest that binding of heparin or mimetics thereof to pro-heparanase causes a change in the conformation, thus may expose the proteolytic sites of H60 to cleavage and allow its activation.

### Example 4

### Identification of Proheparanse Activation Inhibitors Using a Cell-Based Assay

In order to screen a plurality of putative pro-heparanase activation inhibitors the present inventors have developed a cell-based assay, which allows the identification of compound inhibitors which are active in a physiological environment.

Materials and Experimental Procedures

Pro-heparanase activation in a cell-based assay—293 cells (human kidney cells) were grown in DMEM media supplemented with 10% FCS. The cells were plated onto a 96 well tissue culture plate (Costar cat. #3596), at a density of 24,000 cells/well. The plates were incubated for 24 hrs. in a 37° C. humidified incubator with 5% CO₂. Following 24 hrs in culture, 500 ng/well of purified recombinant human pro heparanase (H60, described in U.S. Pat. No. 6,475,763) was applied to the cells. The plates were further incubated for another 4 hrs. The pro-heparanase was allowed to internalize by the cells and further processed to generate the active 53 KDa heparanase.

At the end of the incubation, the cells were lysed by three consecutive cycles of freezing and thawing in a −80° C. Freezer and a 37° C. shaker. Formation of active heparanase was detected by determination of heparanase activity in DMB assay as described in patent application U.S. Pat. No. 6,190,875B.

Screening of chemical compounds for inhibition of pro-heparanase activation—A proprietary chemical library (Insight Biopharmaceuticals Ltd. Israel) was screened for inhibition of pro-heparanase activation at a concentration of ca. 7.5 μM in the cell-based assay described above. Compounds exhibiting inhibition of more than 80% of the positive control (containing 1% DMSO instead of the compounds), were considered as hits. IC50 was calculated from inhibition curves made for all hits. These compounds are listed in Tables 7-13, below.

### Example 5

### Elucidating Mode of Action for the Different Pro-Heparanase Activation Inhibitors

Targets for the different inhibitory compounds, which were identified using the cell-based assay of Example 4, above, were elucidated while considering a number of mechanisms of action, as follows:

1. Compounds which bind to pro-heparanase to thereby interfere with the interaction of heparin or heparan sulfate with its binding sites.

2. Compounds which directly bind to heparin and inhibit binding of the latter to heparanase.

3. Compounds which inhibit Cathepsin B.

4. Compounds which inhibit serine or aspartic proteases.

5. Compounds, which bind pro-heparanase at the protease-binding site to thereby, interfere with pro-heparanase processing.

6. Compounds which interfere with the hetero-dimerization of the heparanase subunits.

Experimental Procedures

Heparin binding—5 μM of the selected compound was prepared in a solution of PBS pH 7.2 with 0.05% Tween and 5% DMSO. 30 μl of either 50% Sepharose CL-6B beads or 50% Heparin sepharose CL-6B beads (Pharmacia Cat No. 17-0467-01) in water, were dispensed into 96 well filter plates (Millipore Cat. # MADVN-065). Excess water was discarded by filtration using a Multiscreen vacuum manifold (Millipore, Cat No. MAVM 096 00). 200 μl of each compound tested was transferred to three wells of the filter plate—one empty well, one with sepharose CL-6B and one with heparin sepharose CL-6B. Plates were rotated for 2 hrs at 37° C. followed by filtration to a new 96 well clear plate, using a Multiscreen vacuum manifold. The absorbance spectrum of the filtrate in each well was determined using a VERSAmax plate reader (Molecular devices). Percent of heparin binding was calculated from the binding to heparin sepharose CL-6B as compared to the binding of the compound to the control sepharose CL-6B beads.

Elisa assay for determination of binding of pro-heparanase to heparin—96 well microplates were coated overnight with Heparin-albumin (Lizard) and blocked with 1% BSA in PBS with 0.05% Tween and 0.01% thimerosal for 2 hrs. Purified pro-heparanase (H60) was added to the plates at a concentration of 50 ng/ml and incubated for 45 min at 23-25° C.

The amount of H60 bound to heparin was detected using goat anti-heparanase polyclonal biotinylated antibodies (application for patent 26872) diluted 1:300 and neutravidin HRP (Pierce cat. #31001) diluted 1:7000. TMB (Pierce Cat. #34021) was used as a substrate for peroxidase. The plates were read at 450 nm with a reference wavelength of 630 nm using a VERSAmax plate reader. For determination of inhibition of β-FGF (Sigma, F0291) binding, compounds in 1% DMSO, were pre-incubated with H60 prior to application to the plates, for 45 min at 23-25° C. Percent inhibition was calculated as compared with the control reactions pre-incubated with 1% DMSO only.

Transient transfections—293 cells (200,000) were plated onto a 6 well tissue culture plate. 24 hrs later, the cells were transfected with 1 μg pSI (Promega) hpa plasmid (U.S. Pat. No. 6,475,763) in 3 μl Fugene reagent that was pre-incubated for 15 min in 100 μl medium without serum. Cells were treated with inhibitors 4 and 17 hrs following transfection.

Cathepsin B activity assay—Cathepsin B activity was determined using the quenched fluorescent peptide substrate (Z-Arg-Arg-Amino-methyl coumarin, Calbiochem Cat. #219392). The activity was determined in reaction mixtures containing Acetate buffer pH 5.5 with 4 mM EDTA, and 8 mM freshly prepared DTT, 100 μM substrate and 60 mU cathepsin B (Calbiochem Cat. No. 219364), The reaction was incubated for 20 minutes at 37° C. Fluorescence was read using a FLUOstar Galaxy fluorometer with an excitation at 390 nm and emission at 460 nm. For determination of inhibition of cathepsin B activity, the tested compound was diluted in 1% DMSO and added to the reaction mixture. Protease activity in the presence of the inhibitor was compared to control reaction mixture containing 1% DMSO.

Elisa assay for determination of binding of β-FGF to heparin—96 well microplates were coated overnight with Heparin-albumin (Lizard) and blocked with 1% BSA in PBS with 0.05% Tween and 0.01% thimerosal for 2 hrs. β-FGF was added to the plates at a concentration of 0.8 ng/ml and incubated for 45 min at 23-25° C. The amount of β-FGF bound to heparin was detected using anti-FGF monoclonal antibody (Sigma Cat. # F6162) at a concentration of 2.5 ng/ml and secondary antibodies Sheep anti-mouse IgG linked to horseradish peroxidase (1:3000). TMB (Pierce Cat. #34021) was used as a substrate for peroxidase.

The plates were read at 450 nm with a reference wavelength of 630 nm using a VERSAmax plate reader. For determination of inhibition of β-FGF binding, compounds in 1% DMSO, were pre-incubated with β-FGF prior to application to the plates, for 45 min. at 23-25° C. Percent inhibition was calculated as compared with the control reactions pre-incubated with 1% DMSO only.

Elisa assay for determination of binding of VEGF to heparin—96 well microplates were coated overnight with Heparin-albumin (Lizard) and blocked with 1% BSA in PBS with 0.05% Tween and 0.01% thimerosal for 2 hrs. VEGF was added to the plates at a concentration of 2 ng/ml and incubated for 45 min. at 23-25° C. The amount of VEGF bound to heparin was detected using anti-VEGF monoclonal antibody (Sigma Cat. # V4758) at a concentration of 0.26 μg/ml and secondary antibodies Sheep anti-mouse IgG linked to horseradish peroxidase (1:2000). TMB (Pierce Cat. #34021) was used as a substrate for peroxidase.

The plates were read at 450 nm with a reference wavelength of 630 nm using a VERSAmax plate reader. For determination of inhibition of VEGF binding, compounds in 1% DMSO, were pre-incubated with VEGF prior to application to the plates, for 45 min. at 23-25° C. Percent inhibition was calculated as compared with the control reactions pre-incubated with 1% DMSO only.

Elisa assay for determination of binding of antithrombin to heparin—96 well microplates were coated overnight with Heparin-albumin (Lizard) and blocked with I % BSA in PBS with 0.05% Tween and 0.01% thimerosal for 2 hrs. VEGF was added to the plates at a concentration of 0.5 μg/ml and incubated overnight at 4° C. The amount of antithrombin bound to heparin was detected using an anti-antithrombin monoclonal antibody (Sigma Cat. # A5816) diluted to 1:200, and secondary antibodies (anti-mouse anti-Fc? conjugated to horseradish peroxidase, Jackson cat. # 115-035-008, 1:100). TMB (Pierce Cat. #34021) was used as a substrate for peroxidase.

The plates were read at 450 nm with a reference wavelength of 630 nm using a VERSAmax plate reader. For determination of inhibition of antithrombin binding, compounds in 1% DMSO, were pre-incubated with antithrombin prior to application to the plates, for 45 min. at 23-25° C. Percent inhibition was calculated as compared to the control reactions pre-incubated with 1% DMSO only.

Determination of elastase activity—Elastase activity was determined using the synthetic peptide MeOSuc-AAPV-AMC (Calbiochem 324740) as a substrate. Elastase, human neutrophil (Calbiochem cat.# 324681) at a final concentration of 9 nM was assayed in a reaction mixture containing 100 mM Tris-HCl pH 7.5 with 500 mM NaCl and 1 μM substrate. The plate was read at time zero for determination of background and then incubated for 150 min at 23-25° C. At the end of the incubation, the plate was read using a POLARstar Galaxy fluorometer with an excitation at 390 nm and emission at 460 nm. For determination of inhibition of elastase activity, the tested compound was added at a final concentration of 1% before the incubation. The percent inhibition was calculated as compared to the positive control containing 1% DMSO.

Determination of urokinase activity—Urokinase activity was determined using the synthetic peptide Z-GGR-AMC (Calbiochem cat.# 672159) as a substrate. Urokinase, human urine (Calbiochem cat.# 672081) at a final concentration of 6 nM was assayed in a reaction mixture containing 100 mM Tris-HCl pH 7.5 with 500 mM NaCl and 1 μM substrate. The plate was read at time zero for determination of background and then incubated for 150 min. at 23-25° C. At the end of the incubation the plate was read using a POLARstar Galaxy fluorometer with an excitation at 390 nm and emission at 460 nm. For determination of inhibition of urokinase activity, the tested compound was added at a final concentration of 1% before the incubation. The percent inhibition was calculated as compared to the positive control containing 1% DMSO.

Determination of chymase activity—Chymase activity was determined using the synthetic peptide N-Succinyl-AAPF-p-mitroanilide (Sigma cat.# S-7388) as a substrate. Chymase, human skin (Calbiochem cat. # 230780) at a final concentration of 100 ng/ml was assayed in a reaction mixture containing 300 mM Tris-HCl pH 8 with 1.5 M NaCl, 0.005% Tween 20 and 2.5 mM substrate. The plate was read at time zero for determination of background and then incubated for 4 hrs at 23-25° C. At the end of incubation, absorbance was determined using a VERSAmax plate reader at 410 nm. For determination of inhibition of chymase activity, the tested compound was added at a final concentration of 1% before the incubation. The percent inhibition was calculated as compared to the positive control containing 1% DMSO.

Results

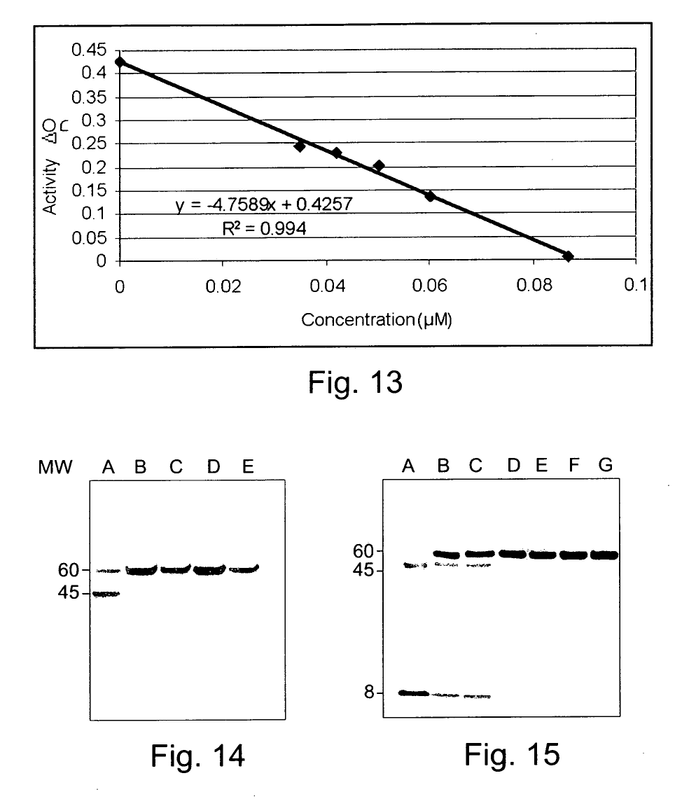

Screening of a chemical library for pro-heparanase activation inhibitors using the above-described cell-based assay, led to the identification of a group of small molecules, which inhibit pro heparanase activation, through different modes of action. IC50 was determined for all selected compounds by checking inhibition curves in the cell-based assay. One example (compound 63) of an IC50 curve is shown in FIG. 13.

In the cell-based assay the pro-heparanase is applied externally to the cells. To ascertain that the compounds inhibit pro-heparanase in a biological system more relevant to the natural process, the ability of three potent compounds (i.e., compound 5, 63 and 112) to inhibit the activation of pro-heparanase transiently expressed in 293 cells was assayed. As is shown in FIG. 14, all three compounds tested, completely inhibited pro-heparanase processing but did not affect pro-heparanase expression.

These compounds also inhibited the processing (FIG. 15) and activation (FIG. 16) of pro-heparanase in-vitro by cathepsin B and cathepsin D in the presence of heparin. Inhibition of processing was also detected when other activating proteases were used (data not shown).

The different compounds were classified to four groups according to their mechanism of action:

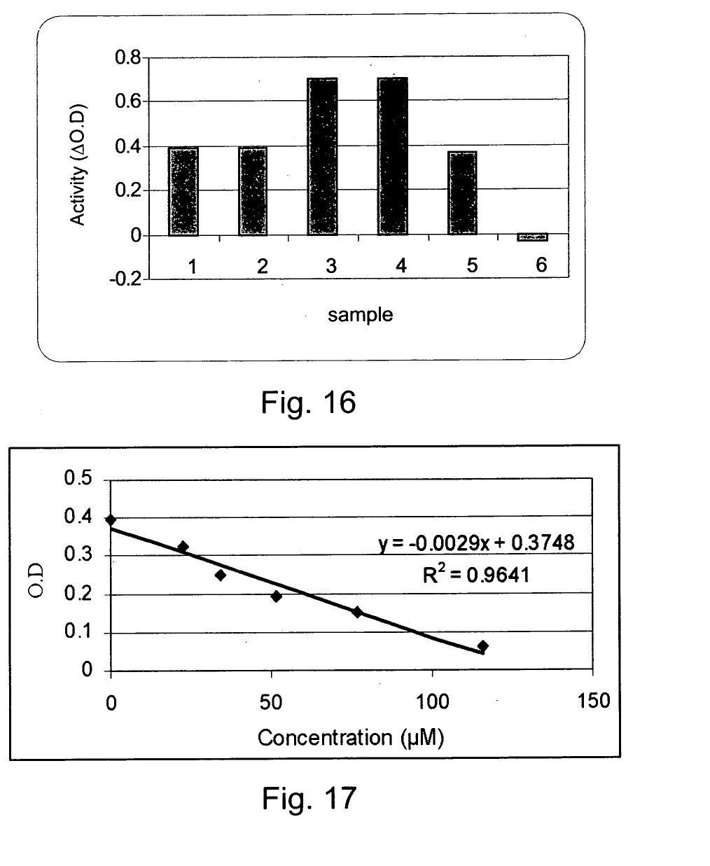

1. Small molecules that bind to one or more of the heparin-binding domains (e.g. PRRKTAKM, SKRRKLRV and QKKFKN) and interfere with the interaction of heparin or heparan sulfate with its binding sites. Binding of these compounds prevents the change in conformation of the pro-heparanase caused due to interaction with heparan sulfate, and thus indirectly inhibits the proteolytic activity. Tables 7-10 illustrate compounds operating through this mode of action. These compounds were shown to inhibit binding of H60 to heparin in an Elisa assay (FIG. 17), but do not directly bind to heparin (Table 15). Direct interaction of compounds 5 and 63 with the pro-heparanase polypeptide was determined using surface plasmon resonance. Both compounds were found to reversibly bind to the H60 molecule (data not shown). Circular dichroism experiments showed that compound 5 prevents the change in conformation of H60 induced by heparin (data not shown).

FIGS. 19a-c model binding of one of the compounds (i.e., compound 5) belonging to this category, to each of the three heparin binding domains of pro-heparanase. It can be observed from the possible binding modes of compound 5 with the three heparin-binding domains of human heparanase that important pharmacophoric elements for hydrogen bonding with the basic residues in the heparin-domains may be the rhodanine carbonyl group; the furan oxygen and the carboxylic acid moiety.

These models are in good correlation with the structure-activity relationships in these series of compounds.

All compounds operating through this mode of action are rhodanine derivatives. The most potent heparanase activation inhibitors (IC50H60<1 μM) that were discovered are among this group. The rhodanine scaffold appears in various compounds with biological activity such as 13-lactamase inhibitors (Grant E B, Bioorg. Med. Chem Lett., 10, 2179, 2000), hepatitis C virus protease inhibitors (Sing W T, Bioorg. Med. Chem. Lett., 11, 91, 2001), aldose reductase inhibitors (Ohishi Y, Chem. Pharm. Bull., 38, 1911, 1990), antifungal agents (Orchard M G, WO 02/022612), sialyl Lewis X synthesis inhibitors (Kobayashi K, JP 11302280), VEGF antagonists (WO 98/53790), phospholipase D (US 04/0002526) and PIN-1 inhibitors (WO 04/028535).

Structure-activity relationships in the rhodanine series (see Tables 7-9, below):

The most potent rhodanine analogs (see Table 7, below) can be classified into three main groups depending on the structural properties of the R1 substituent:
a. R1 bearing an acidic group (i.e., compounds 1-35)—It can be observed that the chain length between the rhodanine group and the acidic moiety is crucial for optimal activity. The best activity is obtained when the chain length is n>=3 (e.g. see compounds 2 and 6). Decreasing the chain length to n=2 or n=1 in a homologous series of compounds causes a decrease in activity (e.g. see compounds 9 and 16). The nature of the acidic moiety (e.g. carboxylic acid or sulphonic acid) does not affect the biological activity (e.g. see compounds 9 and 33). Introduction of an additional acidic group to R1 is deleterious for the biological activity (e.g. see compounds 11 and 12).
b. R1 with a tetrahydro-thiophene-1,1-dioxide group (e.g compounds 59 and 61)
c. R1 with a 1,5-dimethyl-2-phenyl-1,2-dihydro-pyrazol-3-one group (e.g. compounds 63-72)

Compounds with other less favorable R1 groups still maintain reasonable biological activity (e.g. see compounds 36, 38, 41, 42, 47, 52 and 58).

Introducing hydrophobic groups in the 5-position of the furan group seems to be crucial for improving the biological activity (e.g. see compound 63; Table 7 and compound 76; Table 8). For optimal activity, electron-withdrawing substituents (e.g. Cl, Br, NO₂) in meta or para position on the 5-phenyl ring are preferred. In contrast, substituents in ortho positions are less favorable (e.g. see compounds 3, 4 and 5).

It will be appreciated that replacement of the furan group with a thiophene ring does not affect the biological activity (e.g. see compounds 76 and 78). It is further anticipated that replacement of the furan ring with another aryl or heteroaryl group will not harm the activity (e.g. see compound 79; Table 8 and compound 91; Table 9).

Although the rhodanine group seems to be quite essential for biological activity, substitution of this moiety with similar ring systems does not cause an essential decrease in the biological activity (see Table 10). A typical example of such a similar ring system is thio (or oxo)-dihydropyrimidine-4,6-dione (e.g. see compounds 40 and 104).

Introducing bulky groups into these ring systems does not substantially affect the biological activity (e.g. see compounds 92, 93 and 99, 100 and 111). Therefore, it seems that not all the features of these ring systems interact with the target protein and consequently are not essential for biological activity.

TABLE 7
SAR of 5-(5-Phenyl-furan-2-ylmethylene)rhodanine analogs

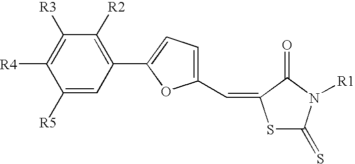

| | | | | | | | | IC50 |
|---|---|---|---|---|---|---|---|---|
| | | | | | | | | H60 |
| | Compound | Supplier | R1 | R2 | R3 | R4 | R5 | (uM) |
| | STOCK1S-85197 | InterBioScreen | (CH₂)₅COOH | H | Cl | H | H | 0.58 |
| 2 | STOCK1S-66348 | InterBioScreen | (CH₂)₅COOH | H | H | Cl | H | 0.52 |
| 3 | STOCK4S-61604 | InterBioScreen | (CH₂)₃COOH | NO₂ | H | H | H | 14.3 |
| 4 | STOCK4S-33449 | InterBioScreen | (CH₂)₃COOH | H | NO₂ | H | H | 0.16 |
| 5 | STOCK1S-89897 | InterBioScreen | (CH₂)₃COOH | H | H | NO₂ | H | 0.13 |
| 6 | STOCK1S-67661 | InterBioScreen | (CH₂)₃COOH | H | H | Cl | H | 0.87 |
| 7 | 1499-0870 | ChemDiv | (CH₂)₂COOH | H | H | H | H | 2.3 |
| 8 | 1503-0685 | ChemDiv | (CH₂)₂COOH | Cl | H | H | H | 6 |
| 9 | 1503-0687 | ChemDiv | (CH₂)₂COOH | Cl | H | Cl | H | 3 |
| 10 | 1503-0690 | ChemDiv | (CH₂)₂COOH | NO₂ | H | H | H | 30 |
| 11 | 0687-1671 | ChemDiv | (CH₂)₂COOH | H | H | NO₂ | H | 2.8 |
| 12 | STOCK1S-81660 | InterBioScreen | CH(COOH)— | H | H | NO₂ | H | 15 |
| | | | CH₂COOH | | | | | |
| 13 | CHS0230647 | ChemStar | CH₂COOH | H | H | H | H | 1.4 |
| 14 | 1058-0112 | ChemDiv | CH₂COOH | Cl | H | H | H | 32 |
| 15 | 2388-1214 | ChemDiv | CH₂COOH | H | Cl | H | H | 2 |
| 16 | 1013-0134 | ChemDiv | CH₂COOH | H | H | Cl | H | 3.8 |
| 17 | CHS0230645 | ChemStar | CH₂COOH | Cl | H | H | Cl | 20 |
| 18 | 1013-0141 | ChemDiv | CH₂COOH | Cl | H | Cl | H | 18 |
| 19 | 0806-0219 | ChemDiv | CH₂COOH | H | Cl | Cl | H | 19 |
| 20 | 2000-06146 | Tripos | CH₂COOH | Cl | H | NO₂ | H | 29 |
| 21 | 0423-0164 | ChemDiv | CH₂COOH | H | H | Br | H | 47 |
| 22 | 1013-0138 | ChemDiv | CH₂COOH | H | H | I | H | 27 |
| 23 | 1013-0136 | ChemDiv | CH₂COOH | H | H | F | H | 5 |
| 24 | 0851-0572 | ChemDiv | CH₂COOH | NO₂ | H | H | H | 37 |
| 25 | 2151-0441 | ChemDiv | CH₂COOH | Me | H | NO₂ | H | 21 |
| 26 | 1013-0137 | ChemDiv | CH₂COOH | H | CF₃ | H | H | 7 |
| 27 | 0806-0218 | ChemDiv | CH₂COOH | H | COOH | H | H | 46 |
| 28 | 1013-0133 | ChemDiv | CH₂COOH | H | H | COOH | H | 22 |
| 29 | 1499-0872 | ChemDiv | CH₂COOH | 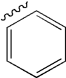 | | NO₂ | H | 64 |
| 30 | 0423-0162 | ChemDiv | CH₂COOH | 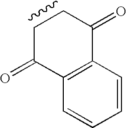 | | H | H | 28 |
| 31 | 1611-2457 | ChemDiv | CH(ipropyl)COOH | H | H | Br | H | 3.6 |
| 32 | STOCK1S-83120 | InterBioScreen | (CH₂)₂SO₃H | H | Cl | H | H | 6 |
| 3 | STOCK1S-70034 | InterBioScreen | (CH₂)₂SO₃H | H | H | Cl | H | 1.8 |
| 34 | STOCK1S-68566 | InterBioScreen | (CH₂)₂SO₃H | H | Cl | Cl | H | 4 |
| 35 | STOCK1S-73374 | InterBioScreen | (CH₂)₂SO₃H | H | NO₂ | H | H | 41 |
| 36 | 0687-1670 | ChemDiv | H | H | H | Br | H | 13 |
| 37 | STOCK2S-54169 | InterBioScreen | H | H | Cl | Me | H | 18 |
| | 1588-0362 | ChemDiv | Me | H | Br | H | H | 5.4 |
| | 1013-0288 | ChemDiv | Me | H | COOH | H | H | 8.5 |
| | 1013-0287 | ChemDiv | Me | H | H | COOH | H | 6.6 |
| | 0806-0289 | ChemDiv | allyl | H | COOH | H | H | 8 |
| | 2027-0309 | ChemDiv | C₆H₁₁ | H | COOH | H | H | 1 |
| | 2027-0312 | ChemDiv | C₆H₁₁ | H | H | COOH | H | 11 |
| | 1013-0169 | ChemDiv | C₆H₅ | Cl | Cl | H | H | 129 |
| 46 | 0930-0241 | ChemDiv | C₆H₅ | Cl | H | NO₂ | H | 250 |
| 47 | 0806-0255 | ChemDiv | C₆H₅ | H | COOH | H | H | 5 |
| 48 | CHS0529892 | ChemStar | C₆H₅ | H | H | COOH | H | 0.95 |
| 49 | 1499-0862 | ChemDiv | C₆H₅ | 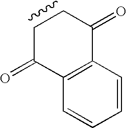 | | H | H | 5 |
| | 2027-0174 | ChemDiv | 3-F—C₆H₅ | H | COOH | H | H | 4 |
| | 2027-0175 | ChemDiv | 3-F—C₆H₅ | H | H | COOH | H | 2 |
| | 2027-0438 | ChemDiv | 3-CF₃—C₆H₅ | H | COOH | H | H | 4 |
| | 2027-0441 | ChemDiv | 3-CF₃—C₆H₅ | H | H | COOH | H | 3 |
| | 1588-0155 | ChemDiv | 3-Cl—C₆H₅ | H | COOH | H | H | 6 |
| | 1588-0182 | ChemDiv | 3-Cl—C₆H₅ | H | H | COOH | H | 2.5 |
| 56 | 2027-0219 | ChemDiv | 3-NO₂—C₆H₅ | H | H | COOH | H | 13 |
| 57 | STOCK1S-73872 | InterBioScreen | CH(COOH)— | H | H | NO₂ | H | 7 |
| | | | (CH₂)₂CH₃ | | | | | |
| 58 | STOCK1S-20916 | InterBioScreen | (CH₂)₃—OMe | H | H | Cl | H | 0.92 |
| 59 | STOCK3S-41076 | InterBioScreen | 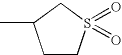 | Cl | H | H | H | 0.24 |
| 60 | 1504-0110 | ChemDiv | 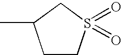 | H | NO₂ | H | H | 1.35 |
| | 1503-0731 | ChemDiv | 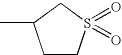 | H | H | NO₂ | H | 0.64 |
| | 2000-13371 | Tripos | 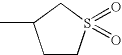 | H | H | NH₂S O₂ | H | 14 |
| | STOCK1S-27757 | InterBioScreen | 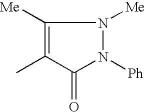 | H | H | Cl | H | 0. |
| | STOCK1S-21087 | InterBioScreen | 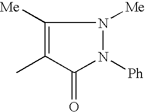 | H | H | Br | H | 0.13 |
| | STOCK1S-28532 | InterBioScreen | 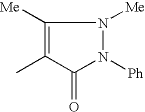 | H | H | NO₂ | H | 0.30 |
| | STOCK3S-44454 | InterBioScreen | 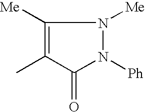 | H | Cl | H | H | 0.18 |
| | STOCK1S-29032 | InterBioScreen | 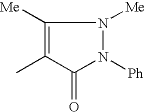 | H | NO₂ | H | H | 0.98 |
| | STOCK1S-25561 | InterBioScreen | 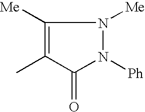 | H | CF₃ | H | H | 0.52 |
| | STOCK1S-21503 | InterBioScreen | 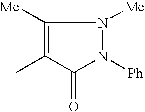 | Cl | H | H | H | 0.19 |
| | STOCK1S-28009 | InterBioScreen | 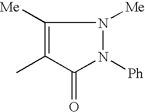 | NO₂ | H | H | H | 0.74 |
| | STOCK3S-34888 | InterBioScreen | 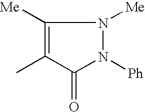 | Cl | Cl | H | H | 0.46 |
| | STOCK1S-23249 | InterBioScreen | 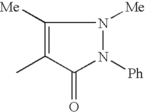 | Cl | H | Cl | H | 0.40 |
| | STOCK1S-73069 | InterBioScreen | 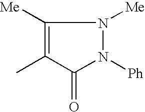 | Cl | H | H | Cl | 3 |
| | STOCK3S-33061 | InterBioScreen | 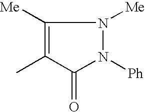 | H | Cl | Cl | H | 1.3 |

TABLE 8
SAR of 5-(Heteroaryl-2-ylmethylene)rhodanine analogs

| | | | | | | | IC50 H60 |
|---|---|---|---|---|---|---|---|
| | Compound | Supplier | A | R1 | R2 | R3 | (uM) |
| 75 | STOCK4S-48559 | InterBioScreen | O | (CH₂)₃COOH | H | 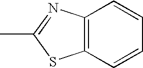 | 4.2 |
| 76 | STOCK1S-49740 | InterBioScreen | O | 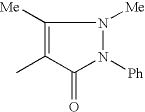 | H | H | 52.0 |
| 77 | STOCK3S-35943 | InterBioScreen | O | 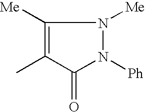 | H | Me | 60.0 |
| 78 | STOCK1S-26193 | InterBioScreen | S | 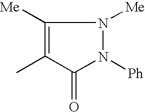 | H | H | 52.0 |
| 79 | STOCK1S-74777 | InterBioScreen | S | 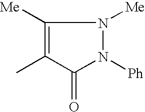 | Me | H | 34.0 |

TABLE 9
SAR of 5-Benzylidene-rhodanine analogs

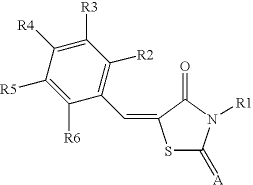

| | | | | | | | | | | IC50 |
|---|---|---|---|---|---|---|---|---|---|---|
| | | | | | | | | | | H60 |
| | Compound | Supplier | A | R1 | R2 | R3 | R4 | R5 | R6 | (uM) |
| | 1207-0080 | ChemDiv | S | Me | OH | H | H | Cl | H | 8.5 |
| | 1207-0083 | ChemDiv | S | Me | OH | H | H | I | H | 13.8 |
| | 1588-0108 | ChemDiv | S | Et | H | NO₂ | OH | H | H | 29 |
| | 1207-0175 | ChemDiv | S | allyl | OH | H | H | Cl | H | 8.5 |
| 84 | 1503-1275 | ChemDiv | S | (CH₂)₅CO₂H | H | H | Br | H | H | 118 |
| 85 | STOCK1S-27665 | InterBioScreen | S | (CH₂)₁₀CO₂H | H | Br | H | H | H | 8 |
| 86 | 1988-0245 | ChemDiv | S | 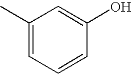 | H | H | H | H | H | 9 |
| 87 | 1674-0041 | ChemDiv | S | 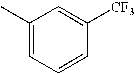 | Cl | H | Cl | H | H | 39 |
| 88 | 1503-0725 | ChemDiv | S | 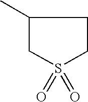 | H | H | NO₂ | H | H | 103 |
| 89 | STOCK2S-46019 | InterBioScreen | S | 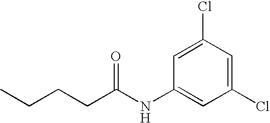 | H | H | Me | H | H | 2 |
| 90 | 1611-4731 | ChemDiv | S | 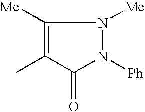 | Cl | H | H | H | F | 57 |
| 91 | STOCK1S-82761 | InterBioScreen | S | 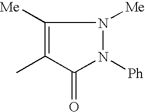 | H | H | Cl | H | H | 12 |
| 92 | 1988-0222 | ChemDiv | 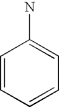 | H | H | OH | H | H | H | |
| 93 | 0928-0161 | ChemDiv | 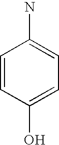 | 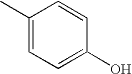 | H | H | H | H | H | 20 |

TABLE 10
SAR of 5-(Furan-2-ylmethylene)-2-(thio)oxo-dihydro-pyrimidine-4,6-dione analogs

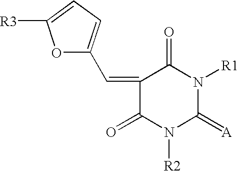

| | | | | | | | IC50 |
|---|---|---|---|---|---|---|---|
| | | | | | | | H60 |
| | Compound | Supplier | A | R1 | R2 | R3 | (uM) |
| | CHS0280354 | ChemStar | S | H | H | 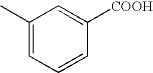 | 23 |
| | CHS0570955 | ChemStar | S | H | H | 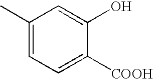 | 6 |
| | 94-00053 | Tripos | S | H | H | 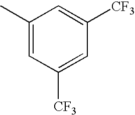 | 11 |
| | STOCK2S-48911 | InterBioScreen | S | 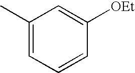 | H | 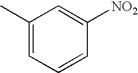 | 9 |
| | STOCK2S-31096 | InterBioScreen | S | 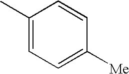 | H | 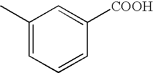 | 58 |
| | 0927-0084 | ChemDiv | S | Ph | Ph | 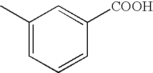 | 3.5 |
| | 1016-0019 | ChemDiv | S | Ph | Ph | 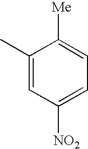 | 42 |
| 101 | 2144-0733 | ChemDiv | O | H | H | 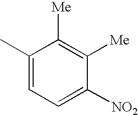 | 23 |
| 102 | 2082-0179 | ChemDiv | O | H | H | 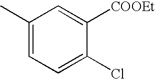 | 40 |
| 103 | 1611-4714 | ChemDiv | O | Me | Me | 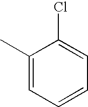 | 49 |
| 104 | CHS0570954 | ChemStar | O | Me | Me | 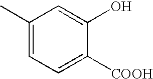 | 18 |
| 105 | 1102-0032 | ChemDiv | O | 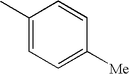 | H | H | 5 |
| 106 | 2368-0068 | ChemDiv | O | 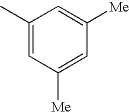 | H | 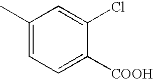 | 25 |
| 107 | 1014-0097 | ChemDiv | O | 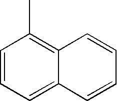 | H | H | 14 |
| 108 | STOCK2S-24348 | InterBioScreen | O | 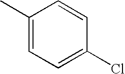 | H | 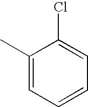 | 19 |
| 109 | 2368-0240 | ChemDiv | O | 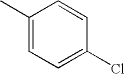 | H | 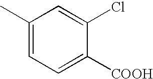 | 19 |
| 110 | STOCK2S-35158 | InterBioScreen | O | 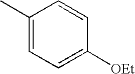 | H | 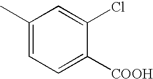 | 2.3 |
| 111 | 0327-0151 | ChemDiv | O | 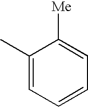 | 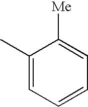 | H | 9 |

2. Small molecules that directly bind to heparin or heparan sulfate and thus prevent them from interacting with the H60 to cause a change in conformation that would allow proteolytic cleavage of the pro-heparanase. Table 11 illustrates compounds that inhibit through this mechanism. These compounds inhibit binding of H60 to heparin in an Elisa assay (as group 1), but were all shown to bind directly to heparin (Table 11).

The molecules that inhibit H60 activation through direct binding to heparin have common chemical features (see Table 11). These molecules are positively charged and planar due to the combination of conjugated bonds and aromatic groups.

Heparin shares common structural characteristics with DNA, since heparin is also negatively charged and its three-dimensional structure is also helical. Therefore, the compounds that bind to heparin have properties (e.g. positively charged and planar) similar to the well-known DNA-intercalators (J E Scott and I H Willett, Nature, 209, 985, 1966).

TABLE 11

| | | | | Binding to | IC50 |
|---|---|---|---|---|---|
| | | | | heparin | H60 |
| | Compound | Supplier | Structure | (5 uM) | (uM) |
| 112 | 341088 | Sigma | 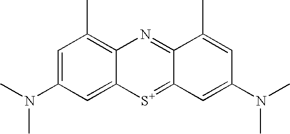 | 70% | 0.57 |
| 113 | 2324-0380 | ChemDiv | 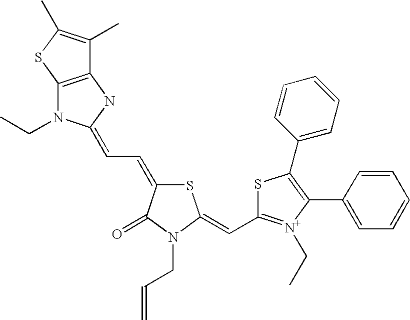 | 73% | 2.61 |
| 114 | 2324-0036 | ChemDiv | 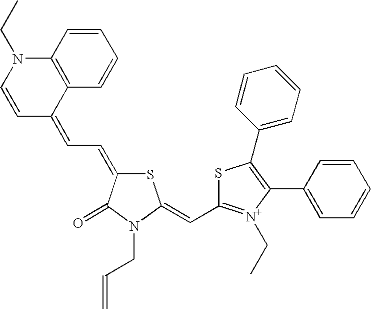 | 48% | 7.00 |
| 115 | 2324-0034 | ChemDiv | 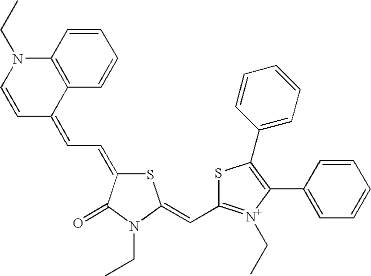 | 66% | 12.00 |
| 116 | 1541-0004 | ChemDiv | 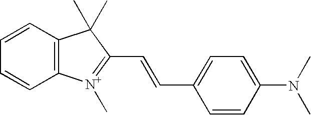 | 71% | 3.00 |
| 117 | 1556-0393 | ChemDiv | 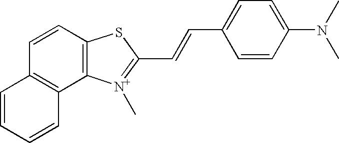 | 66% | 9.00 |
| 118 | 1118-0013 | ChemDiv | 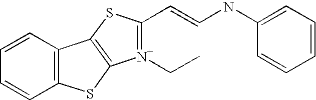 | 61% | 6.00 |
| 119 | 2324-0329 | ChemDiv | 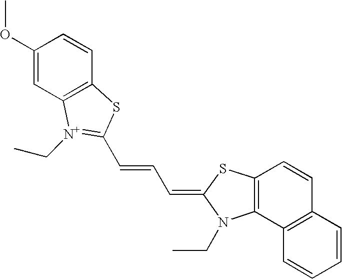 | 51% | 1.74 |
| 120 | 2324-0082 | ChemDiv | 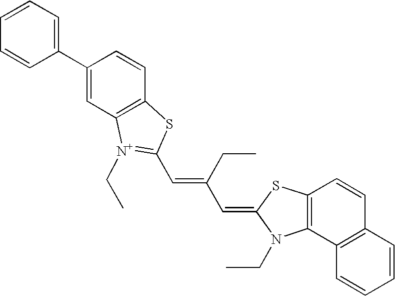 | 61% | 3.50 |
| 121 | 2324-0215 | ChemDiv | 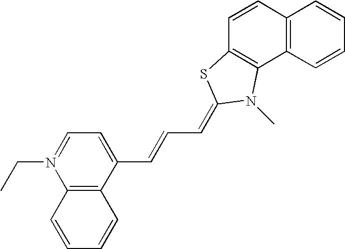 | 47% | 5.27 |
| 122 | 0099-0221 | ChemDiv | 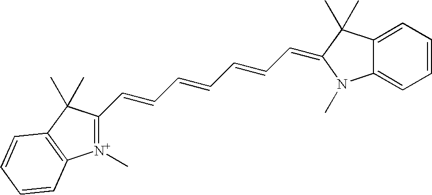 | 65% | 16.00 |

3. Inhibitors of cathepsin B activity—Cathepsin B was shown to process the pro-heparanase at the C-terminus of the H8 subunit of the pro-heparanase. Inhibitors operating through this mechanism directly prevent processing of the pro-heparanase to yield an active enzyme. Table 12 lists known inhibitors of cathepsin B activity and their IC50 for inhibition of pro-heparanase activation.

TABLE 12

| | Compound | | | | IC50 H60 |
|---|---|---|---|---|---|
| | (Inhibitor) | Protease | Supplier | Structure | (uM) |
| 123 | A6185 | Calpain I Calpain II Cathepsin B Cathepsin L | Sigma | 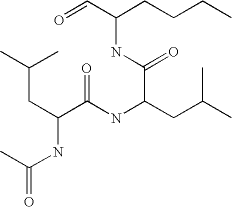 | 7.4 |
| 124 | A6060 | Calpain I Calpain II Cathepsin B Cathepsin L | Sigma | 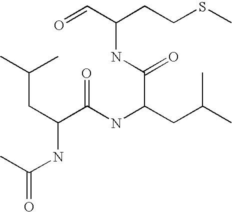 | 10.0 |
| 125 | 205531 | Cathespin B | Calbiochem | 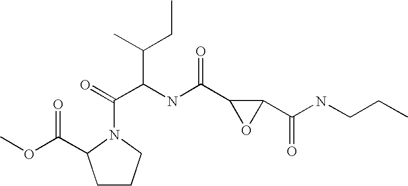 | 23.0 |
| 126 | L9783 | Cysteine Proteases Trypsin-like Serine Proteases | Sigma | 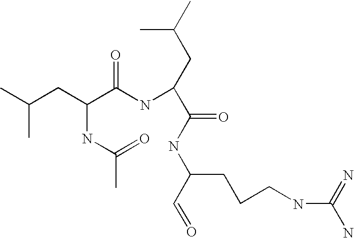 | 26.0 |
| 127 | E3132 | Cysteine Proteases | Sigma | 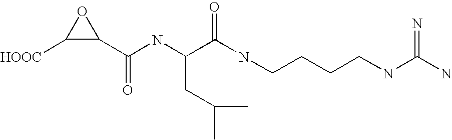 | 32.0 |

4. Small molecules that inhibit a serine protease that cleaves the pro heparanase at the N-terminus of the H45 polypeptide and thus prevent its activation. Compound 128 belonging to this group was shown to inhibit pro-heparanase activation in-vitro by cathepsin G (a serine protease) but not by cathepsin B (a cysteine protease) and cathepsin D (an aspartic protease) (FIG. 18). Both activation assays were performed in the presence of heparin. Compound 128 was found to inhibit the activity of cathepsin G (data not shown). These results indicate that this group of compounds do not inhibit pro-heparanase activation by interference with heparin binding, or by inhibition of cathepsin B, but are likely to inhibit a serine protease that cleaves the pro heparanase at the N-terminus of the H45 polypeptide. The SAR of these compounds and their selectivity towards inhibition of serine proteases belonging to the three different subclasses, trypsin-like (urokinase), chymotrypsin-like (chymase) and elastase, are presented in Table 13 below. All compounds that inhibited pro-heparanase activation through this mechanism were found to inhibit elastase activity but not chymase or urokinase activity (data not shown) indicating that the protease that cleaves the pro-heparanase at the n-terminus of the H45 polypeptide is elastase-like (see FIGS. 5 and 6).

TABLE 13
SAR and selectivity of serine protease inhibitors

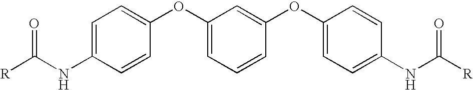

| | | | | IC50 | IC50 | IC50 | IC50 |
|---|---|---|---|---|---|---|---|
| | | | | H60 | Urokinase | Chymase | Elastase |
| | Compound | Supplier | R | (uM) | (uM) | (uM) | (uM) |
| 128 | 8002-7182 | ChemDiv | 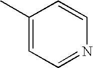 | 0.50 | NI | NI | 13.0 |
| 129 | 8002-7186 | ChemDiv | 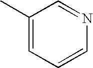 | 59 | NI | NI | 2.4 |
| 130 | STOCK 1S-05875 | InterBioScreen | 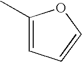 | 10 | NI | NI | 28.4 |
| 131 | 0089-0028 | ChemDiv | 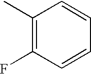 | | NI | NI | 5.4 |
| 132 | 2372-3863 | ChemDiv | 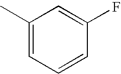 | 1 | NI | NI | 4.6 |
| 133 | 0627-0418 | ChemDiv | 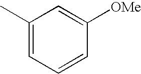 | 74 | NI | NI | 3.1 |
| | 2000-01602 | Tripos | 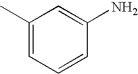 | 38 | NI | NI | 10.0 |
| | 1706-0313 | ChemDiv | 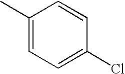 | 4 | NI | NI | 2.1 |
| 136 | 0632-0854 | ChemDiv | 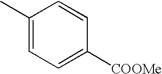 | 49 | NI | NI | 2.7 |
| 137 | 0868-0280 | ChemDiv | 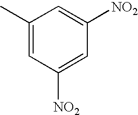 | 3 | NI | NI | 1.2 |

NI = no inhibition

Some of the compounds that inhibit heparin binding were found to have a dual effect. In addition to their inhibition of pro-heparanase activation they also inhibit heparanase activity. Table 15 presents results of IC50 towards inhibition of pro-heparanase activation (H60) and heparanase activity inhibition (H53). From the structure analysis of these inhibitors, it is obvious that only compounds with an acidic group show dual activity (see compounds 1,4,5,6,42).

Heparin and heparan sulfate are unique among glycosaminoglycans in their ability to bind to a large number of different (primarily extracellular) proteins. The binding of heparinoids by many of these proteins results in a modification of the protein's activity or metabolism. These interactions play important roles in normal physiological as well as pathological processes.

Antithrombin (AT III) is a serine protease inhibitor that inactivates several members of the blood coagulation cascade, the most important of which are thrombin and factor Xa. The inhibition of these proteases by AT III is accelerated due to binding of heparin by a factor of 2000. Initial heparin binding to ATIII is a low-affinity interaction that causes a conformational change (Villanueva G. B. and Danishefsky I., Biochem. Biophys. Res. Commun., 74, 803-809, 1977) in the structure of ATIII, which promotes additional stronger binding to heparin and facilitates the reaction of the target protease with ATIII (Capila I. and Linhardt. J. Angew. Chem. Int. Ed., 41, 390-412, 2002).

Fibroblast growth factors (FGFs) are members of a large family of proteins involved in developmental and physiological processes including cell proliferation, differentiation, morphogenesis and angiogenesis. FGFs are heparin-binding proteins that have a high affinity for cell surface HSPGs. These growth factors exert their biological effects by binding to different, specific cell surface receptors called fibroblast growth factor receptors (FGFRs). The FGFRs are also heparin-binding proteins, thus, the three compounds FGF, FGFR, and heparan sulfate must interact simultaneously to initiate signal (Capila I. and Linhardt J., Angew. Chem. Int. Ed. 2002, 41, 390-412).

The initial binding of a virus to a target cell often represents a critical step in pathogenesis. Binding may result from a receptor-like interaction between a viral coat protein and a heparan sulfate chain of a proteoglycans expressed on the surface of the target cell. Heparan sulfate has an essential role in steps leading to HIV membrane fusion and in some cases also acts as the cell surface receptor for HIV-1 (Mondor I. et. al, J. Virol. 72, 3623-3634, 1988).

Other viruses such as herpes simplex virus, Dengue virus, respiratory syncytial virus, cytomegalovirus, the adeno-associated virus and the foot-and-mouth disease virus, all employ heparan sulfate in their initial step of infection (Capila I. and Linhardt J., Angew. Chem. Int. Ed., 41, 390-412, 2002).

The interaction of heparin and heparan sulfate with adhesion proteins has implications in various physiological and pathological processes including inflammation (Varki A., Proc. Nat. Acad. Sci. 91, 7390-7397, 1994), nerve tissue growth (Ruavala H., EMBO J. 8, 2933-2941, 1989), tumor cell invasion, and plaque formation in the brain of Alzheimer patients (Buee L. et al. Brain res. 601, 154-163, 1993) and in prion disease (Chadha K. C. et al., BioChromatography 2, 211-223, 1997).

The role of various heparin-binding proteins in different physiological and pathological processes is summarized in Table 14, below (adapted from Capila I. and Linhardt J., Angew. Chem. Int. Ed., 41, 390-412, 2002).

TABLE 14

| | | Function of | |
|---|---|---|---|
| | Physiological/Pathological role | heparin binding | References |
| ATIII | Coagulation cascade serpin | enhances | 1 |
| SLPI | Inhibits elastase and cathepsin G | enhances | 2 |
| C1 INH | Inhibits C1 esterase | enhances | 3 |
| VCP | Protects host cells from complement | unclear | 4 |
| FGF | Cell proliferation, differentiation, | activates signal | 5 |
| | morphogenesis and angiogenesis | transduction | |
| PF-4 | Inflammation and wound healing | inactivates heparin | 6 |
| IL-8 | Pro-inflammatory cytokine | promotes | 7 |
| SDF-1a | Pro-inflammatory mediator | localizes | 8 |
| Annexin II | Receptor for TPA and plasminogen, | unclear | 9 |
| | CMV and tenascin C | | |
| Annexin V | Anticoagulant activity, influenza and | assembles | 10 |
| | Hepatitis B viral entry | | |
| ApoE | Lipid transport, AD risk factor | localizes | 11 |
| HIV-1 gp120 | Viral entry | inhibits | 12 |
| CypA | Viral localization and entry | inhibits | 13 |
| T at | Transactivating factor, primes cells | antagonizes | 14 |
| | for HIV infection | | |
| HSV gB and gC | Viral entry into cell | inhibits | 15 |
| HSV gD | Viral entry and fusion | inhibits | 16 |
| Dengue virus envelope pro | Viral localization | inhibits | 17 |
| Malaria CS protein | | | |
| Selectins | Sporozoite attachment to hepatocytes | inhibits | 18 |
| Vitronectin | Adhesion, inflammation, metastasis | blocks | 19 |
| Fibronectin | Cell adhesion and migration | removes | 20 |
| HB-GAM | Adhesion and traction | reorganizes | 21 |
| AP | Nuerite outgrowth in development | mediates | 22 |
| | In amyloid plaque | assembles | 23 |

Inhibitors that prevent interaction of the heparin or heparan sulfate with the heparin-binding domain either through interaction with one or more of the heparin-binding domains or by direct binding to glycosaminoglycans, were tested for their ability to inhibit other heparin-binding proteins-fibroblast growth factor (β-FGF), vascular endothelial growth factor (VEGF) and antithrombin.

The results are presented in Table 15, below. Compounds operating through binding to the heparin-binding domain were found to inhibit both β-FGF and VEGF binding to heparin but had no effect on binding of antithrombin to heparin. Some compounds, such as compounds 58 and 59 exhibited sub-micromolar inhibition. These inhibitors may be advantageous, as they may not only inhibit cell migration, invasion and indirect neovascular response through inhibition of heparanase activation, but may also directly inhibit the induction of neovascularization in pathological situations through inhibition of VEGF and β-FGF. (Vlodavsky I et al; Cell. Molec. Aspects, Acad. Press. Inc. pp. 327-343, 1993, Thunberg L et al; FEBS. Lett., 117, 203-206,1980).

These inhibitors may be useful for treatment of other diseases in which heparin-binding proteins have a crucial role such as inflammation (e.g. selectins), cardiovascular diseases (e.g. lipoprotein lipases), central nervous system diseases (e.g. beta-amyloid, prion proteins) and viral diseases (e.g. viral attachment proteins such as gp120).

The lack of inhibition of antithrombin binding to heparin may prevent interference with the anticoagulant action of heparin through the heparin/antithrombin complexation which leads to the inactivation of both thrombin and factor Xa. This may be an advantage over the use of heparinomimetic molecules that exhibit less selectivity and may inhibit antithrombin activation and thus may cause a potentially harmful effect of thrombosis at non-injured sites in the vascular wall.

TABLE 15

| | | | | | | % Binding |
|---|---|---|---|---|---|---|
| | IC50 H60 | IC50 H53 | IC50 FGF | IC50 VEGF | IC50 ATIII | to heparin |
| Compound | (uM) | (uM) | (uM) | (uM) | (uM) | (uM) |
| 1 | 0.58 | 10.0 | 5.8 | 4.2 | NI | |
| 4 | 0.16 | 3.0 | 53.4 | 17.8 | NI | |
| 5 | 0.13 | 12.5 | 6.1 | 3.2 | NI | |
| 6 | 0.87 | 36.0 | 23.9 | 17.8 | NI | |
| 42 | 1.00 | 68.0 | 16.3 | 10.8 | NI | |
| 58 | 0.92 | NI | 0.2 | 0.2 | NI | |
| 59 | 0.24 | NI | 0.4 | 0.4 | NI | |
| 63 | 0.07 | NI | 2.2 | 1.2 | NI | |
| 64 | 0.13 | NI | 1.7 | 1.0 | NI | |
| 68 | 0.52 | NI | 2.6 | 2.6 | NI | |
| 71 | 0.46 | NI | 1.7 | 3.8 | NI | |
| 112 | 0.57 | 20.0 | 1.3 | 2.4 | 18.0 | 70 |

NI = no inhibition

### Example 6

### H45, H8 and H6-Derived Heparanase Inhibitors

Experimental Procedures

Peptide synthesis—Peptides were synthesized by the peptide unit of the Weizmann Institute of Science, Rehovot, Israel.

Dimerization assay—Activation assay using di-methyl-methylene-blue was effected as described in U.S. Pat. No. 6,190,875.

H60 activation assay—see Example 4 above.

Results

Tables 16 and 17 list peptides derived from the sequence of H8 and H45 respectively, and abilities thereof to inhibit H53 hetero-dimer formation.

TABLE 16

| | SEQ | | IC50 | IC50 |
|---|---|---|---|---|
| | ID | | H53 | H60 |
| Name | NO: | Sequence | (μM) | (μM) |
| Pep-7 | 16 | CPAYLRFGGTKTDFLIFDPLLE | 7 | ND |
| P8-4 | 17 | PRFLILLGSPKLR | 2.3 | NI |
| P8-5 | 18 | LRTLARGLSPAYL | 18 | NI |
| P8-8 | 19 | FLILLGSPKLRTL | 8 | ND |
| P8-11 | 20 | PRFLILL | 1.4 | 65 |
| P8-16 | 31 | DPRFLILL | 6 | ND |
| P8-18 | 32 | RFLILL | 5 | ND |

NI - no inhibition
ND - not determined

TABLE 17

| | | | IC50 |
|---|---|---|---|
| | | | Heparanase |
| Name | SEQ ID NO: | Sequence | (μM) |
| Pep-25 | 21 | CTWHHYYLNGRTATR | 21 |

Table 18, below lists H6-C-Terminus derived peptides, which may be used to inhibit pro-heparanase activation.

TABLE 18

| SEQ ID NO: | Sequence |
|---|---|
| 22 | EQLLLREHYQ-Electrophile |
| 23 | QLLLREHYQ-Electrophile |
| 24 | LLLREHYQ-Electrophile |
| 25 | LLREHYQ-Electrophile |
| 26 | LREHYQ-Electrophile |
| 27 | REHYQ-Electrophile |
| 28 | EHYQ-Electrophile |
| 29 | HYQ-Electrophile |
| 30 | YQ-Electrophile |

TABLE 19

| | Name | Electrophile | Mechanism |
|---|---|---|---|
| 1 | Aldehydes | —COH | Reversible |
| 2 | Boronates | —BO₂H₂ | Reversible |
| | Nitriles | —CN | Reversible |
| 4 | β-Lactams | 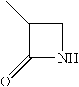 | Reversible |
| | Vinyl Sulfones | 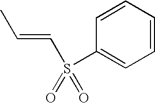 | Irreversible |
| | Epoxides | 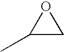 | Irreversible |
| 7 | Halomethylketones | —COCH₂Cl | Irreversible |
| | Isocoumarins | 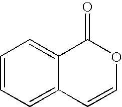 | Irreversible |
| | Thiadiazoles | 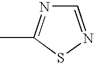 | Irreversible |

### Example 7

### Effect of Compounds 5, 6 and 59 on Cell Migration, Invasion and Cord Formation

The inhibitory effect of compounds which interfere with heparin binding to pro-heparanase was tested on several cellular functions which are mediated by heparanase and other heparin-binding proteins (see Table 14, above).

Materials and Experimental Procedures

In vitro assay of invasion inhibition—The ability of the compounds of the invention to inhibit cell invasion was determined quantitatively by the in vitro Endothelial Cell Migration assay using a BD BioCoat Angiogenesis System kit. The kit consisted of a 24-multiwell insert plate (FluoroBlok, BD Falcon) containing a microporous (3.0 μm pore size) polyethylene terephthalate (PET) membrane that was capable of blocking fluorescence completely (>99% efficiency). This membrane was uniformly coated with matrigel (BD Matrigel Matrix). The uniform layer of matrigel matrix served as a reconstituted authentic basement membrane in vitro, providing a true barrier to non-invasive cells, but allowing endothelial cells to attach to the membrane and freely migrate towards an angiogenic stimulus in the lower chamber of the insert plate. Post-labeling the cells with a fluorescent dye and measuring the fluorescence of invading cells in a fluorescent plate reader, provided quantitative measurement of cell invasion.

Each of the tested compounds was diluted to a concentration that was found to be non-toxic to the HT1080 cells. To cover the optimal seeding density for HT1080 cells, suspensions containing various cell concentrations were prepared: 1 ml of 3×10⁵ cells/ml, 8 ml of 1.5×10⁵ cells/ml and 1 ml of 0.75×10⁵ cells/ml. The top chambers of each well in the inserts were filled with 0.25 ml cell-suspension, 750 μM DMEM containing 5% fetal calf serum and an inhibitor solution. The plates were incubated for 22 hours at 37° C. and 8% CO₂ atmosphere. At the end of incubation, the medium was aspirated from the upper chambers, and the insert was transferred into a second 24-well plate containing 0.5 ml/well of the fluorescent dye Calcein AM solution (4 μg/ml per plate, prepared from 50 μg Calcein AM dissolved in 20 μl DMSO and 12.5 ml of warm HBSS medium), and incubated for 90 minutes at 37° C., 8% CO₂ atmosphere. Fluorescence of invaded cells was read in a fluorescence plate reader (POLARstar, Galaxy, BMG) with bottom read capabilities at excitation/emission wavelength of 485/530 nm, without further manipulation. Only those labeled cells that have invaded the matrigel and passed through the pores of the PET membrane, were detected. Since the fluorescent blocking membrane effectively blocked the passage of light from 490-700 nm, fluorescence from cells that have not invaded the membrane was blocked from detection.

Cord formation and cell migration assays were executed following explicit directives provided by the inventors of the present invention by the National Cancer Institute (Biological Testing Branch, Developmental Therapeutics Program, DCTD, NCI, Fairview Center, Suite 205, 1003 West 7th Street, Frederick, Md. 21701-8527).

Cord Formation Assay—Matrigel (60 μl of 10 mg/ml; Collaborative Lab # 35423) was placed in each well of an ice-cold 96-well plate. The plate was incubated at room temperature for 15 minutes and then transferred to 37° C. for 30 minutes to permit matrigel polymerization. Concomitantly with plate coating, HUVEC cells were diluted in EGM-2 (Clonetic # CC3162) at a concentration of 2×10⁵ cells/ml. Each test compound was prepared at a 2 fold higher concentration than desired (5 concentration levels) using the same medium. Cells (500 μl) and 2× drug (500 μl) were mixed and 200 μl of this suspension were placed in duplicates on the polymerized matrigel. Following 24 h incubation, triplicate pictures were taken for each concentration using a Bioquant Image Analysis system. Drug effect (IC₅₀) was assessed by comparing to untreated controls and measuring the length of cords formed and number of junctions.

Cell Migration Assay—Migration was assessed using a 48-well Boyden chamber including 8 μm pore size collagen-coated (10 μg/ml rat tail collagen; Collaborative Laboratories) polycarbonate filters (Osmonics, Inc.). The bottom chamber wells included 27-29 μl of DMEM medium alone (baseline) or medium containing chemo-attractant (bFGF, VEGF or Swiss 3T3 cell conditioned medium). The top chambers included 45 μl of HUVEC cell suspension (1×10⁶ cells/ml) prepared in DMEM+1% BSA with or without the test compound. Followinf 5 h of incubation at 37° C., the membrane was rinsed with PBS, fixed and stained in Diff-Quick solutions. The filter was placed on a glass slide with the migrated cells facing down and cells on top were removed using a Kimwipe. The testing was performed in 4-6 replicates where five fields were counted from each well. Negative unstimulated control values were subtracted from stimulated control and drug treated values. Data wad plotted as mean migrated cell±S.D. IC₅₀ was calculated from the plotted data.

Results

Table 20, below, lists IC₅₀ values for inhibition of cellular functions which are associated with the activities of heparanase and other heparin-binding proteins, such as bFGF and VEGF, by compounds which inhibit heparin binding thereto.

TABLE 20

| | Inhibition of | Inhibition of | Inhibition of |
|---|---|---|---|
| | invasion | cord formation | chemotaxis |
| Compound | IC50 (μM) | IC50 (μM) | IC50 (μM) |
| 5 | 12.0 | 3.9 | 0.4 |
| 6 | ND | 3.2 | 4.0 |
| 59 | ND | 0.4 | 0.25 |

ND = not determined

### Example 8

### Anti Tumorigenic Effect of Compound 5 as Determined Using an In-Vivo Xenogeneic Model

Once the mechanism of action of the anti-heparanase compounds of the present invention was determined, the present inventors addressed their anti-tumorigenic effect.

Materials and Experimental Procedures

Mpanc-96 tumor cell injection—MPanc-96 pancreatic tumor cells were obtained from the ATCC(CCL-2380). On day 1, cells (5×10⁶ cells/0.2 ml RPMI+FBS) were subcutaneously injected to the right flank, midway between the axillary and inguinal regions, of SCID mice. Prior to injection the cells were mixed by gentle vortexing. The cells were injected using a 25G needle, and the appearance of a bulge was observed. Following injection to animals, cells were counted in the leftover tubes with trypan blue, and re-cultured to test proliferation.

Compound 5 injections—Tumors were measured prior to inhibitor injection. Inhibitor injection was commenced when 100% of the animals had a visible tumor (about 6 days following tumor cells injection). The animals were injected intraperitoneally, daily, twice a day, starting 6 days after tumor cells injection, with 50 μl plain DMSO or 50 μl of Compound 5 in DMSO (8 mg/ml in DMSO).

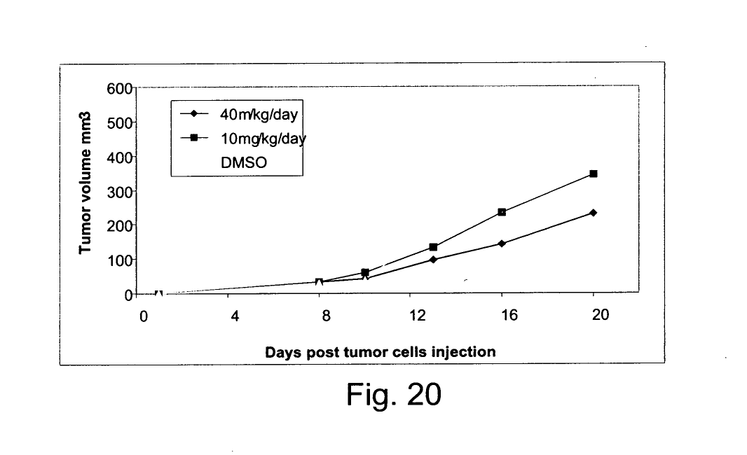

Tumor growth—Tumor size was measured in two perpendicular dimensions, twice a week using digital calipers. Volumes were estimated using the formula (α²×β)/2, where α is the shorter of the two dimensions, and β is the longer of the two dimensions. The mean group's tumor growth rate is shown in FIG. 20.

Study termination—At the end of the study, 21 days following tumor cell injection, animals were euthanized by IP veterinary penthal injection. In case of severe weight loss (>20%), a tumor>1000 mm³ or depression, an animal was euthanized prior to the study's termination day. Upon animal death prior to the end of the study, body weight and tumor size were measured.

Results

MPanc-96 pancreatic tumor cells which have high intracellular heparanase activity were injected into SCID mice. Once having a predetermined tumor size, animals were injected with compound 5.

FIG. 20 shows tumor volume in the presence or absence of compound 5. In the absence of compound 5, tumor volume reached 500 mm² following 20 days of study. In the presence of compound 5, a significant smaller tumor volume was apparent. This reduction was dose dependent, as a dose of 40 mg/kg/day of compound 5 reduced tumor volume by 35% while twice as much reduction was apparent upon treatment with 10 mg/kg/day of compound 5.

These results support a therapeutic efficacy for the compounds of the present invention.

As can be seen from Table 20 a good correlation exists between inhibition of pro-heparanase activation, FGF and VEGF binding to heparin (presented in Table 15) and inhibition of invasion, cord formation and chemotaxis. The in-vitro cellular assays described in this example, represent in-vivo processes involved in cancer and inflammation. The in-vitro assay for invasion serves as an indicator of metastasis, the cord formation assay for angiogenesis, and the chemotaxis assay for inflammation. The results shown herein may indicate that these compounds may be useful in inhibition of cancer through two different mechanisms: I. Inhibition of primary tumor development through inhibition of angiogenesis II. Inhibition of metastasis through inhibition of invasion. In addition these inhibitors may be useful in treating inflammation through inhibition of chemotaxis.

Furthermore, these results indicate that screening for inhibition of pro-heparanase activation using the cell-based assay described in example 4, may lead to detection of compounds which may be developed into agents for treating cancer and inflammation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of regulating heparanase activity in a tissue, the method comprising modulating heparanase activation, thereby regulating heparanase activity in the tissue.

2. The method of claim 1, wherein said modulating heparanase activation is effected by:
(a) modulating activity of at least one protease participating in pro-heparanase activation;
(b) modulating heparin binding to pro-heparanase; and/or
(c) modulating heparanase dimerization.

3. The method of claim 1, wherein said modulating heparanase activation is inhibiting heparanase activation.

4. The method of claim 1, wherein said modulating heparanase activation is increasing heparanase activation.

5. The method of claim 2, wherein said protease is selected from the group consisting of a serine protease, a cysteine protease and an aspartic protease.

6. The method of claim 5, wherein said serine protease is elastase or cathepsin G.

7. The method of claim 5, wherein said cysteine protease is cathepsin B.

8. The method of claim 5, wherein said aspartic protease is cathepsin D.

9. The method of claim 3, wherein said inhibiting said heparanase activation is effected by:
(i) an agent capable of inhibiting at least one protease participating in said pro-heparanase activation;
(ii) an agent capable of inhibiting binding of heparin to pro-heparanase; and/or
(iii) an agent capable of inhibiting heparanase heterodimerization.

10. The method of claim 9, wherein said agent capable of inhibiting at least one protease participating in said pro-heparanase activation is selected from the group consisting of a cysteine protease inhibitor, an aspartic protease inhibitor and a serine protease inhibitor.

11. The method of claim 10, wherein said cysteine protease inhibitor is selected from the group consisting of CA074, CA074Me, E-64, Cathepsin B inhibitor I (Z-Phe-Ala-CH₂F-A), Cathepsin B inhibitor II (Ac-Leu-Val-lysinal), Leupeptin, Leupeptin analogs, Cathepsin inhibitor I (Phe-Gly-NHO-Bz), Cathepsin inhibitor II (Phe-Gly-NHO-Bz-pMe), Cathepsin inhibitor III (Phe-Gly-NHO-Bz-pOme), Calpain inhibitor I (ALLN, N-Acetyl-Leu-Leu-NIe-CHO) and Calpain inhibitor II (ALLM, N-Acetyl-Leu-Leu-Met-CHO).

12. The method of claim 10, wherein said aspartic protease inhibitor is a cathepsin D inhibitor or a cathepsin E inhibitor each selected from the group consisting of Pepstatin A, Pepstatin A Me and a −2macroglobulin.

13. The method of claim 10, wherein said serine protease inhibitor is a compound having the general formula: 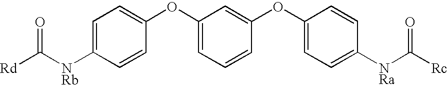
wherein:
Ra and Rb are each independently selected from the group consisting of hydrogen, alkyl, cycloalkyl or aryl; and
Rc and Rd are each independently selected from the group consisting of a substituted and unsubstituted aryl and a substituted and unsubstituted heteroaryl.

14. The method of claim 13, wherein each of Rc and Rd is a heteroaryl.

15. The method of claim 14, wherein said heteroaryl is 3-pyridine.

16. The method of claim 13, wherein each of Rc and Rd is a substituted aryl.

17. The method of claim 16, wherein said substituted aryl is a phenyl substituted by an electron withdrawing group.

18. The method of claim 9, wherein said agent capable of inhibiting at least one protease participating in said pro-heparanase activation is a peptide.

19. The method of claim 18, wherein said peptide is derived from SEQ ID NO: 36.

20. The method of claim 19, wherein said peptide is as set forth in SEQ ID NO: 22, 23, 24, 25, 26, 27, 28, 29 or 30.

21. The method of claim 18, wherein said peptide is conjugated to an electrophilic group.

22. The method of claim 21, wherein said electrophilic group is derived from a chemical group selected from the group consisting of aldehydes, boronates, nitriles, β-lactams, vinyl sulfones, epoxides, halomethylketones, isocoumarin and thiodiazoles.

23. The method of claim 9, wherein said agent capable of inhibiting binding of heparin to pro-heparanase, is a heparin-binding agent.

24. The method of claim 9, wherein said agent capable of inhibiting binding of heparin to pro-heparanase, is a proheparanse binding agent.

25. The method of claim 23, wherein said heparin binding agent is a planar, positively charged compound.

26. The method of claim 25, wherein said planar, positively charged compound is selected from the group of compounds listed in Table 11.

27. The method of claim 24, wherein said pro-heparanase binding agent is a compound having the general formula: 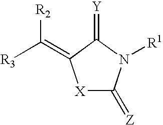
wherein:
X is O, S, NR₄ or NR₅—C(=D);
Y and Z are each independently O, S or NR₄;
R₁ is selected from the group consisting of hydrogen, a substituted or unsubstituted alkyl, a substituted or unsubstituted cycloalkyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
—(CH₂)n-CH(R₆)-Q₁(OH);
and
R₂ and R₃ are each independently selected from the group consisting of hydrogen, a substituted or unsubstituted aryl and a substituted or unsubstituted heteroaryl, at least one of R₂ and R₃ being said substituted or unsubstituted aryl or heteroaryl,
and wherein:
D is O, S or NR₄;
R₄ and R₅ are each independently selected from the group consisting of hydrogen, a substituted or unsubstituted alkyl, a substituted or unsubstituted cycloalkyl and a substituted or unsubstituted aryl;
n is integer that equals 0-20;
R₆ is selected from the group consisting of hydrogen, alkyl and Q₂(OH); and
Q₁ and Q₂ are each inependently selected from the group consisting of C═O and S(═O)₂,
whereas each of said substituted alkyl, substituted alkenyl, substituted allyl, substituted cycloalkyl, substituted aryl, substituted heteroaryl and substituted heteroalicyclic independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

28. The method of claim 27, wherein:
X is S;
Y is O; and
Z is S or O.

29. The method of claim 28, wherein Z is S.

30. The method of claim 27, wherein:
X is NR₅—C=D;
Y is O;
Z is O or S; and
D is O or S.

31. The method of claim 27, wherein R₁ is said acid-containing moiety.

32. The method of claim 31, wherein n is greater than 1.

33. The method of claim 32, wherein n equals 2-5.

34. The method of claim 27, wherein said R₁ is a substituted or unsubstituted heteroaryl.

35. The method of claim 34, wherein said heteroaryl is selected from the group consisting of terahydrothiphenyl-1,1-dioxide and 1,5-dimethyl-2-phenyl-1,2-dihydro-3-one-pyrazolyl.

36. The method of claim 27, wherein R₁ is a substituted or unsubstituted aryl.

37. The method of claim 36, wherein said aryl is selected from the group consisting of unsubstituted phenyl, 3-halophenyl, 3-trihalomethylphenyl and 3-nitrophenyl.

38. The method of claim 27, wherein at least one of R₂ and R₃ is a substituted or unsubstituted heteroaryl.

39. The method of claim 38, wherein said heteroaryl has the general formula: 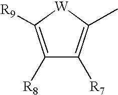
wherein:
W is O or S; and
R₇, R₈ and R₉ are each independently selected from the group consisting of hydrogen, a substituted or unsubstituted alkyl, a substituted or unsubstituted cycloalkyl, a substituted or unsubstituted aryl and a substituted or unsubstituted heteroaryl,
whereas each of said substituted alkyl, substituted cycloalkyl, substituted aryl and substituted heteroaryl independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

40. The method of claim 39, wherein:
R₇ and R₅ are each hydrogen; and
R₉ is an aryl having the general formula: 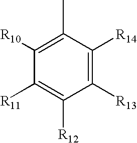
wherein each of R₁₀-R₁₄ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

41. The method of claim 40, wherein:
R₁₀ and R₁₄ are each independently selected from the group consisting of hydrogen, alkyl, cycloalkyl, hydroxy, alkoxy, thiohydroxy, thioalkoxy, aryloxy and thioaryloxy; and
R₁₁-R₁₃ are each independently selected from the group consisting of hydrogen, halo, nitro, trihaloalkyl and C-carboxy.

42. The method of claim 39, wherein:
R₇ and R₈ are each hydrogen; and
R₉ is a substituted or unsubstituted benzothiazole.

43. The method of claim 27, wherein at least one of R₂ and R₃ is a substituted or unsubstituted aryl having the general formula: 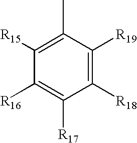
wherein each of R₁₅-R₁₉ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

44. The method of claim 40, wherein:
X is S;
Y is O;
Z is S; and
R₁ is said acid-containing moiety.

45. The method of claim 44, wherein:
n equals 2-5;
Q₁ is C═O; and
Q₂ is hydrogen.

46. The method of claim 44, wherein each of R₁₀-R₁₄ is indpenedently selected from the group consisting of hydrogen, nitro and halo.

47. The method of claim 40, wherein:
X is S;
Y is O;
Z is S; and
R₁ is selected from the group consisting of aryl, alkoxy-substituted alkyl, and heteroaryl.

48. The method of claim 47, wherein each of R₁₀-R₁₄ is indpenedently selected from the group consisting of hydrogen, nitro, C-carboxy and halo.

49. The method of claim 48, wherein at least one of R₁₀-R₁₄ is C-carboxy and said C-carboxy is a carboxylic acid group.

50. The method of claim 48, wherein R₁ is phenyl.

51. The method of claim 48, wherein R₁ is 3-methoxypropyl.

52. The method of claim 48, wherein R₁ is tetrahydrothiphenyl-1,1-dioxide.

53. The method of claim 48, wherein R₁ is 1,5-dimethyl-2-phenyl-1,2-dihydro-3-one-pyrazolyl.

54. The method of claim 9, wherein said agent capable of inhibiting heparanase heterodimerization is a peptide of no more than 50 amino acids.

55. The method of claim 54, wherein said peptide is derived from SEQ ID NO: 33 or 35.

56. The method of claim 55, wherein said peptide is as set forth in SEQ ID NO: 16, 17, 18, 19, 20, 21, 31 or 32.

57. The method of claim 4, wherein said increasing pro-heparanase activation is effected by upregulation of:
(i) at least one protease participating in pro-heparanase activation; and/or
(ii) heparin, heparin mimetic, heparan sulfate and/or heparan sulfate mimetic.

58. A method of regulating a biological process depending at least in part on heparanase activity, the method comprising modulating heparanase activation, thereby regulating the biological process depending at least in part on heparanase activity.

59. The method of claim 58, wherein said modulating heparanase activation is effected by:
(a) modulating activity of at least one protease participating in pro-heparanase activation;
(b) modulating heparin binding to pro-heparanase; and/or
(c) modulating heparanase dimerization.

60. The method of claim 58, wherein said modulating heparanase activation is inhibiting heparanase activation.

61. The method of claim 58, wherein said modulating heparanase activation is increasing heparanase activation.

62. The method of claim 59, wherein said protease is selected from the group consisting of a serine protease, a cysteine protease and an aspartic protease.

63. The method of claim 62, wherein said serine protease is elastase or cathepsin G.

64. The method of claim 62, wherein said cysteine protease is cathepsin B.

65. The method of claim 62, wherein said aspartic protease is cathepsin D.

66. The method of claim 60, wherein said inhibiting said heparanse activation is effected by:
(i) an agent capable of inhibiting at least one protease participating in said pro-heparanase activation;
(ii) an agent capable of inhibiting binding of heparin to pro-heparanase; and/or
(iii) an agent capable of inhibiting heparanase heterodimerization.

67. The method of claim 66, wherein said agent capable of inhibiting at least one protease participating in said pro-heparanase activation is selected from the group consisting of a cysteine protease inhibitor, an aspartic protease inhibitor and a serine protease inhibitor.

68. The method of claim 67, wherein said cysteine protease inhibitor is selected from the group consisitng of CA074, CA074Me, E-64, Cathepsin B inhibitor I (Z-Phe-Ala-CH₂F-A), Cathepsin B inhibitor II (Ac-Leu-Val-lysinal), Leupeptin, Leupeptin analogs, Cathepsin inhibitor I (Phe-Gly-NHO-Bz), Cathepsin inhibitor II (Phe-Gly-NHO-Bz-pMe), Cathepsin inhibitor III (Phe-Gly-NHO-Bz-pOme), Calpain inhibitor I (ALLN, N-Acetyl-Leu-Leu-NIe-CHO) and Calpain inhibitor II (ALLM, N-Acetyl-Leu-Leu-Met-CHO).

69. The method of claim 67, wherein said aspartic protease inhibitor is a cathepsin D inhibitor or a cathepsin E inhibitor each selected from the group consisting of Pepstatin A, Pepstatin A Me and a −2macroglobulin.

70. The method of claim 67, wherein said serine protease inhibitor is a compound having the general formula: 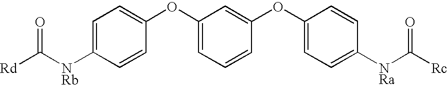
wherein:
Ra and Rb are each independently selected from the group consisting of hydrogen, alkyl, cycloalkyl or aryl; and
Rc and Rd are each independently selected from the group consisting of a substituted and unsubstituted aryl and a substituted and unsubstituted heteroaryl.

71. The method of claim 70, wherein each of Rc and Rd is a heteroaryl.

72. The method of claim 71, wherein said heteroaryl is 3-pyridine.

73. The method of claim 70, wherein each of Rc and Rd is a substituted aryl.

74. The method of claim 73, wherein said substituted aryl is a phenyl substituted by an electron withdrawing group.

75. The method of claim 66, wherein said agent capable of inhibiting at least one protease participating in said pro-heparanase activation is a peptide.

76. The method of claim 75, wherein said peptide is derived from SEQ ID NO: 36.

77. The method of claim 76, wherein said peptide is as set forth in SEQ ID NO: 22, 23, 24, 25, 26, 27, 28, 29 or 30.

78. The method of claim 75, wherein said peptide is conjugated to an electrophilic group.

79. The method of claim 78, wherein said electrophilic group is derived from a chemical group selected from the group consisting of aldehydes, boronates, nitriles, β-lactams, vinyl sulfones, epoxides, halomethylketones, isocoumarin and thiodiazoles.

80. The method of claim 66, wherein said agent capable of inhibiting binding of heparin to pro-heparanase, is a heparin-binding agent.

81. The method of claim 66, wherein said agent capable of inhibiting binding of heparin to pro-heparanase, is a proheparanse binding agent.

82. The method of claim 80, wherein said heparin binding agent is a planar, positively charged compound.

83. The method of claim 82, wherein said planar, positively charged compound is selected from the group of compounds listed in Table 11.

84. The method of claim 81, wherein said pro-heparanase binding agent is a compound having the general formula: 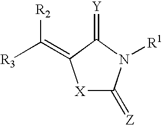
wherein:
X is O, S, NR₄ or NR₅—C(=D);
Y and Z are each independently O, S or NR₄;
R₁ is selected from the group consisting of hydrogen, a substituted or unsubstituted alkyl, a substituted or unsubstituted cycloalkyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
—(CH₂)n-CH(R₆)-Q₁(OH);
and
R₂ and R₃ are each independently selected from the group consisting of hydrogen, a substituted or unsubstituted aryl and a substituted or unsubstituted heteroaryl, at least one of R₂ and R₃ being said substituted or unsubstituted aryl or heteroaryl,
and wherein:
D is O, S or NR₄;
R₄ and R₅ are each independently selected from the group consisting of hydrogen, a substituted or unsubstituted alkyl, a substituted or unsubstituted cycloalkyl and a substituted or unsubstituted aryl;
n is integer that equals 0-20;
R₆ is selected from the group consisting of hydrogen, alkyl and Q₂(OH); and
Q₁ and Q₂ are each inependently selected from the group consisting of C═O and S(═O)₂,
whereas each of said substituted alkyl, substituted alkenyl, substituted allyl, substituted cycloalkyl, substituted aryl, substituted heteroaryl and substituted heteroalicyclic independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

85. The method of claim 84, wherein:
X is S;
Y is O; and
Z is S or O.

86. The method of claim 85, wherein Z is S.

87. The method of claim 84, wherein:
X is NR₅—C=D;
Y is O;
Z is O or S; and
D is O or S.

88. The method of claim 84, wherein R₁ is said acid-containing moiety.

89. The method of claim 88, wherein n is greater than 1.

90. The method of claim 89, wherein n equals 2-5.

91. The method of claim 84, wherein said R₁ is a substituted or unsubstituted heteroaryl.

92. The method of claim 91, wherein said heteroaryl is selected from the group consisting of terahydrothiphenyl-1,1-dioxide and 1,5-dimethyl-2-phenyl-1,2-dihydro-3-one-pyrazolyl.

93. The method of claim 84, wherein R₁ is a substituted or unsubstituted aryl.

94. The method of claim 93, wherein said aryl is selected from the group consisting of unsubstituted phenyl, 3-halophenyl, 3-trihalomethylphenyl and 3-nitrophenyl.

95. The method of claim 84, wherein at least one of R₂ and R₃ is a substituted or unsubstituted heteroaryl.

96. The method of claim 95, wherein said heteroaryl has the general formula: 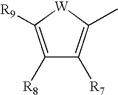
wherein:
W is O or S; and
R₇, R₈ and R₉ are each independently selected from the group consisting of hydrogen, a substituted or unsubstituted alkyl, a substituted or unsubstituted cycloalkyl, a substituted or unsubstituted aryl and a substituted or unsubstituted heteroaryl,
whereas each of said substituted alkyl, substituted cycloalkyl, substituted aryl and substituted heteroaryl independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

97. The method of claim 96, wherein:
R₇ and R₈ are each hydrogen; and
R₉ is an aryl having the general formula: 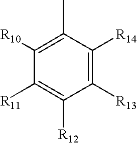
wherein each of R₁₀-R₁₄ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

98. The method of claim 97, wherein:
R₁₀ and R₁₄ are each independently selected from the group consisting of hydrogen, alkyl, cycloalkyl, hydroxy, alkoxy, thiohydroxy, thioalkoxy, aryloxy and thioaryloxy; and
R₁₁-R₁₃ are each independently selected from the group consisting of hydrogen, halo, nitro, trihaloalkyl and C-carboxy.

99. The method of claim 96, wherein:
R₇ and R₈ are each hydrogen; and
R₉ is a substituted or unsubstituted benzothiazole.

100. The method of claim 84, wherein at least one of R₂ and R₃ is a substituted or unsubstituted aryl having the general formula: 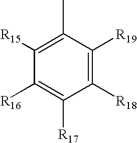
wherein each of R₁₅-R₁₉ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

101. The method of claim 97, wherein:
X is S;
Y is O;
Z is S; and
R₁ is said acid-containing moiety.

102. The method of claim 101, wherein:
n equals 2-5;
Q₁ is C═O; and
Q₂ is hydrogen.

103. The method of claim 101, wherein each of R₁₀-R₁₄ is indpenedently selected from the group consisting of hydrogen, nitro and halo.

104. The method of claim 97, wherein:
X is S;
Y is O;
Z is S; and
R₁ is selected from the group consisting of aryl, alkoxy-substituted alkyl, and heteroaryl.

105. The method of claim 104, wherein each of R₁₀-R₁₄ is indpenedently selected from the group consisting of hydrogen, nitro, C-carboxy and halo.

106. The method of claim 105, wherein at least one of R₁₀-R₁₄ is C-carboxy and said C-carboxy is a carboxylic acid group.

107. The method of claim 105, wherein R₁ is phenyl.

108. The method of claim 105, wherein R₁ is 3-methoxypropyl.

109. The method of claim 105, wherein R₁ is tetrahydrothiphenyl-1,1-dioxide.

110. The method of claim 105, wherein R₁ is 1,5-dimethyl-2-phenyl-1,2-dihydro-3-one-pyrazolyl.

111. The method of claim 66, wherein said agent capable of inhibiting heparanase heterodimerization is a peptide of no more than 50 amino acids.

112. The method of claim 111, wherein said peptide is derived from SEQ ID NO: 33 or 35.

113. The method of claim 112, wherein said peptide is as set forth in SEQ ID NO: 16, 17, 18, 19, 20, 21, 31 or 32.

114. The method of claim 61, wherein said increasing pro-heparanase activation is effected by upregulation of:
(i) at least one protease participating in pro-heparanase activation; and/or
(ii) heparin, heparin mimetic, heparan sulfate and/or heparan sulfate mimetic.

115. The method of claim 58, wherein the biological process is selected from the group consisting of cell migration, cell invasion, cell implantation, cell transplantation, cell extravasation, bone formation, cell adhesion, embryo implantation, neurodegenerative disorders, autoimmune diseases, atherosclerosis, viral infections, restenosis, skeletal muscle calcium kinetics, diabetic nephropathy, epidermal differentiation and desquamation, HS-involved metabolic disorders, prion diseases, hair growth, angiogenesis, neovascularization, cancer development, metastases formation, wound healing, inflammation and immune recognition.

116. A method of treating a heparanase associated disease or disorder in a subject, the method comprising modulating in the subject activation of heparanase, thereby treating the heparanase associated disease or disorder in the subject.

117. The method of claim 116, wherein said modulating heparanase activation is effected by:
(a) modulating activity of at least one protease participating in pro-heparanase activation;
(b) modulating heparin binding to pro-heparanase; and/or
(c) modulating heparanase dimerization.

118. The method of claim 116, wherein said modulating heparanase activation is inhibiting heparanase activation.

119. The method of claim 116, wherein said modulating heparanase activation is increasing heparanase activation.

120. The method of claim 117, wherein said protease is selected from the group consisting of a serine protease, a cysteine protease and an aspartic protease.

121. The method of claim 120, wherein said serine protease is elastase or cathepsin G.

122. The method of claim 120, wherein said cysteine protease is cathepsin B.

123. The method of claim 120, wherein said aspartic protease is selected from the group consisting of cathepsin D and cathepsin E.

124. The method of claim 118, wherein said inhibiting said heparanse activation is effected by:
(i) an agent capable of inhibiting at least one protease participating in said pro-heparanase activation;
(ii) an agent capable of inhibiting binding of heparin to pro-heparanase; and/or
(iii) an agent capable of inhibiting heparanase heterodimerization.

125. The method of claim 124, wherein said agent capable of inhibiting at least one protease participating in said pro-heparanase activation is selected from the group consisting of a cysteine protease inhibitor, an aspartic protease inhibitor and a serine protease inhibitor.

126. The method of claim 125, wherein said cysteine protease inhibitor is selected from the group consisitng of CA074, CA074Me, E-64, Cathepsin B inhibitor I (Z-Phe-Ala-CH₂F-A), Cathepsin B inhibitor II (Ac-Leu-Val-lysinal), Leupeptin, Leupeptin analogs, Cathepsin inhibitor I (Phe-Gly-NHO-Bz), Cathepsin inhibitor II (Phe-Gly-NHO-Bz-pMe), Cathepsin inhibitor III (Phe-Gly-NHO-Bz-pOme), Calpain inhibitor I (ALLN, N-Acetyl-Leu-Leu-NIe-CHO) and Calpain inhibitor II (ALLM, N-Acetyl-Leu-Leu-Met-CHO).

127. The method of claim 125, wherein said aspartic protease inhibitor is a cathepsin D inhibitor or a cathepsin E inhibitor each selected from the group consisting of Pepstatin A, Pepstatin A Me and a −2macroglobulin.

128. The method of claim 125, wherein said serine protease inhibitor is a compound having the general formula: 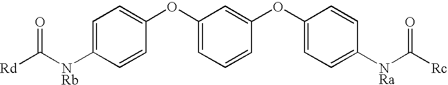
wherein:
Ra and Rb are each independently selected from the group consisting of hydrogen alkyl, cycloalkyl or aryl; and
Rc and Rd are each independently selected from the group consisting of a substituted and unsubstituted aryl and a substituted and unsubstituted heteroaryl.

129. The method of claim 128, wherein each of Rc and Rd is a heteroaryl.

130. The method of claim 129, wherein said heteroaryl is 3-pyridine.

131. The method of claim 128, wherein each of Rc and Rd is a substituted aryl.

132. The method of claim 131, wherein said substituted aryl is a phenyl substituted by an electron withdrawing group.

133. The method of claim 124, wherein said agent capable of inhibiting at least one protease participating in said pro-heparanase activation is a peptide.

134. The method of claim 133, wherein said peptide is derived from SEQ ID NO: 36.

135. The method of claim 134, wherein said peptide is as set forth in SEQ ID NO: 22, 23, 24, 25, 26, 27, 28, 29 or 30.

136. The method of claim 133, wherein said peptide is conjugated to an electrophilic group.

137. The method of claim 136, wherein said electrophilic group is derived from a chemical group selected from the group consisting of aldehydes, boronates, nitrites, β-lactams, vinyl sulfones, epoxides, halomethylketones, isocoumarin and thiodiazoles.

138. The method of claim 124, wherein said agent capable of inhibiting binding of heparin to pro heparanase, is a heparin-binding agent.

139. The method of claim 124, wherein said agent capable of inhibiting binding of heparin to pro heparanase, is a pro-heparanse binding agent.

140. The method of claim 138, wherein said heparin binding agent is a planar, positively charged compound.

141. The method of claim 140, wherein said planar, positively charged compound is selected from the group of compounds listed in Table 11.

142. The method of claim 139, wherein said pro-heparanase binding agent is a compound having the general formula: 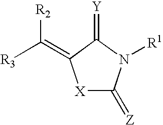
wherein:
X is O, S, NR₄ or NR₅—C(=D);
Y and Z are each independently O, S or NR₄;
R₁ is selected from the group consisting of hydrogen, a substituted or unsubstituted alkyl, a substituted or unsubstituted cycloalkyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
—(CH₂)n-CH(R₆)-Q₁(OH);
and
R₂ and R₃ are each independently selected from the group consisting of hydrogen, a substituted or unsubstituted aryl and a substituted or unsubstituted heteroaryl, at least one of R₂ and R₃ being said substituted or unsubstituted aryl or heteroaryl,
and wherein:
D is O, S or NR₄;
R₄ and R₅ are each independently selected from the group consisting of hydrogen, a substituted or unsubstituted alkyl, a substituted or unsubstituted cycloalkyl and a substituted or unsubstituted aryl;
n is integer that equals 0-20;
R₆ is selected from the group consisting of hydrogen, alkyl and Q₂(OH); and
Q₁ and Q₂ are each inependently selected from the group consisting of C═O and S(═O)₂,
whereas each of said substituted alkyl, substituted alkenyl, substituted allyl, substituted cycloalkyl, substituted aryl, substituted heteroaryl and substituted heteroalicyclic independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

143. The method of claim 142, wherein:
X is S;
Y is O; and
Z is S or O.

144. The method of claim 143, wherein Z is S.

145. The method of claim 142, wherein:
X is NR₅—C=D;
Y is O;
Z is O or S; and
D is O or S.

146. The method of claim 142, wherein R₁ is said acid-containing moiety.

147. The method of claim 146, wherein n is greater than 1.

148. The method of claim 147, wherein n equals 2-5.

149. The method of claim 142, wherein said R₁ is a substituted or unsubstituted heteroaryl.

150. The method of claim 149, wherein said heteroaryl is selected from the group consisting of terahydrothiphenyl-1,1-dioxide and 1,5-dimethyl-2-phenyl-1,2-dihydro-3-one-pyrazolyl.

151. The method of claim 142, wherein R₁ is a substituted or unsubstituted aryl.

152. The method of claim 151, wherein said aryl is selected from the group consisting of unsubstituted phenyl, 3-halophenyl, 3-trihalomethylphenyl and 3-nitrophenyl.

153. The method of claim 142, wherein at least one of R₂ and R₃ is a substituted or unsubstituted heteroaryl.

154. The method of claim 153, wherein said heteroaryl has the general formula: 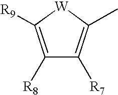
wherein:
W is O or S; and
R₇, R₈ and R₉ are each independently selected from the group consisting of hydrogen, a substituted or unsubstituted alkyl, a substituted or unsubstituted cycloalkyl, a substituted or unsubstituted aryl and a substituted or unsubstituted heteroaryl,
whereas each of said substituted alkyl, substituted cycloalkyl, substituted aryl and substituted heteroaryl independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

155. The method of claim 154, wherein:
R₇ and R₈ are each hydrogen; and
R₉ is an aryl having the general formula: 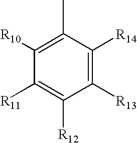
wherein each of R₁₀-R₁₄ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

156. The method of claim 155, wherein:
R₁₀ and R₁₄ are each independently selected from the group consisting of hydrogen, alkyl, cycloalkyl, hydroxy, alkoxy, thiohydroxy, thioalkoxy, aryloxy and thioaryloxy; and
R₁₁-R₁₃ are each independently selected from the group consisting of hydrogen, halo, nitro, trihaloalkyl and C-carboxy.

157. The method of claim 154, wherein:
R₇ and R₈ are each hydrogen; and
R₉ is a substituted or unsubstituted benzothiazole.

158. The method of claim 142, wherein at least one of R₂ and R₃ is a substituted or unsubstituted aryl having the general formula: 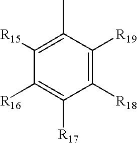
wherein each of R₁₅-R₁₉ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

159. The method of claim 155, wherein:
X is S;
Y is O;
Z is S; and
R₁ is said acid-containing moiety.

160. The method of claim 159, wherein:
n equals 2-5;
Q₁ is C═O; and
Q₂ is hydrogen.

161. The method of claim 159, wherein each of R₁₀-R₁₄ is indpenedently selected from the group consisting of hydrogen, nitro and halo.

162. The method of claim 155, wherein:
X is S;
Y is O;
Z is S; and
R₁ is selected from the group consisting of aryl, alkoxy-substituted alkyl, and heteroaryl.

163. The method of claim 162, wherein each of R₁₀-R₁₄ is indpenedently selected from the group consisting of hydrogen, nitro, C-carboxy and halo.

164. The method of claim 163, wherein at least one of R₁₀-R₁₄ is C-carboxy and said C-carboxy is a carboxylic acid group.

165. The method of claim 163, wherein R₁ is phenyl.

166. The method of claim 163, wherein R₁ is 3-methoxypropyl.

167. The method of claim 163, wherein R₁ is tetrahydrothiphenyl-1,1-dioxide.

168. The method of claim 163, wherein R₁ is 1,5-dimethyl-2-phenyl-1,2-dihydro-3-one-pyrazolyl.

169. The method of claim 124, wherein said agent capable of inhibiting heparanase heterodimerization is a peptide of no more than 50 amino acids.

170. The method of claim 169, wherein said peptide is derived from SEQ ID NO: 33 or 35.

171. The method of claim 170, wherein said peptide is as set forth in SEQ ID NO: 16, 17, 18, 19, 20, 21, 31 or 32.

172. The method of claim 119, wherein said increasing pro-heparanase activation is effected by upregulation of:
(i) at least one protease participating in pro-heparanase activation; and/or
(ii) heparin, heparin mimetic, heparan sulfate and/or heparan sulfate mimetic.

173. The method of claim 115, wherein said heparanase associated disease or disorder is selected from the group consisting of cancer, inflammatory diseases, cardiovascular diseases, neurological diseases and viral diseases

174. A pharmaceutical composition for use in the treatment of heparanase-associated disease or disorder, the pharmaceutical composition comprising a therapeutically effective amount of an agent capable of modulating heparanase activation and a pharmaceutically acceptable carrier or diluent, said pharmaceutical composition is packaged in a packaging material and is identified in print in or on said packaging material for treating the heparanase-associated disease or disorder.

175. The pharmaceutical composition of claim 174, wherein said agent capable of modulating heparanase activation is capable of inhibiting heparanase activation.

176. The pharmaceutical composition of claim 175, wherein said agent capable of inhibiting heparanse activation is:
(i) an agent capable of inhibiting at least one protease participating in said pro-heparanase activation;
(ii) an agent capable of inhibiting binding of heparin to pro-heparanase; and/or
(iii) an agent capable of inhibiting heparanase heterodimerization.

177. The pharmaceutical composition of claim 176, wherein said protease is selected from the group consisting of a serine protease, a cysteine protease and an aspartic protease.

178. The pharmaceutical composition of claim 177, wherein said serine protease is elastase or cathepsin G.

179. The pharmaceutical composition of claim 177, wherein said cysteine protease is cathepsin B.

180. The pharmaceutical composition of claim 177, wherein said aspartic protease is selected from the group consisting of cathepsin D and cathepsin E.

181. The pharmaceutical composition of claim 176, wherein said agent capable of inhibiting at least one protease participating in said pro-heparanase activation is selected from the group consisting of a cysteine protease inhibitor, an aspartic protease inhibitor and a serine protease inhibitor.

182. The pharmaceutical composition of claim 181, wherein said cysteine protease inhibitor is selected from the group consisitng of CA074, CA074Me, E-64, Cathepsin B inhibitor I (Z-Phe-Ala-CH₂F-A), Cathepsin B inhibitor II (Ac-Leu-Val-lysinal), Leupeptin, Leupeptin analogs, Cathepsin inhibitor I (Phe-Gly-NHO-Bz), Cathepsin inhibitor II (Phe-Gly-NHO-Bz-pMe), Cathepsin inhibitor III (Phe-Gly-NHO-Bz-pOme), Calpain inhibitor I (ALLN, N-Acetyl-Leu-Leu-NIe-CHO) and Calpain inhibitor II (ALLM, N-Acetyl-Leu-Leu-Met-CHO).

183. The pharmaceutical composition of claim 181, wherein said aspartic protease inhibitor is a cathepsin D inhibitor or a cathepsin E inhibitor each selected from the group consisting of Pepstatin A, Pepstatin A Me and a −2macroglobulin.

184. The pharmaceutical composition of claim 181, wherein said serine protease inhibitor is a compound having the general formula: 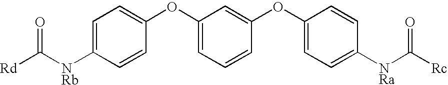
wherein:
Ra and Rb are each independently selected from the group consisting of hydrogen, alkyl, cycloalkyl or aryl; and
Rc and Rd are each independently selected from the group consisting of a substituted and unsubstituted aryl and a substituted and unsubstituted heteroaryl.

185. The pharmaceutical composition of claim 184, wherein each of Rc and Rd is a heteroaryl.

186. The pharmaceutical composition of claim 185, wherein said heteroaryl is 3-pyridine.

187. The pharmaceutical composition of claim 184, wherein each of Rc and Rd is a substituted aryl.

188. The pharmaceutical composition of claim 187, wherein said substituted aryl is a phenyl substituted by an electron withdrawing group.

189. The pharmaceutical composition of claim 176, wherein said agent capable of inhibiting at least one protease participating in sad pro-heparanase activation is a peptide.

190. The pharmaceutical composition of claim 189, wherein said peptide is derived from SEQ ID NO: 36.

191. The pharmaceutical composition of claim 190, wherein said peptide is as set forth in SEQ ID NO: 22, 23, 24, 25, 26, 27, 28, 29 or 30.

192. The pharmaceutical composition of claim 189, wherein said peptide is conjugated to an electrophilic group.

193. The pharmaceutical composition of claim 192, wherein said electrophilic group is derived from a chemical group selected from the group consisting of aldehydes, boronates, nitrites, β-lactams, vinyl sulfones, epoxides, halomethylketones, isocoumarin and thiodiazoles.

194. The pharmaceutical composition of claim 176, wherein said agent capable of inhibiting binding of heparin to pro-heparanase, is a heparin-binding agent.

195. The pharmaceutical composition of claim 176, wherein said agent capable of inhibiting binding of heparin to pro-heparanase, is a proheparanse binding agent.

196. The pharmaceutical composition of claim 194, wherein said heparin binding agent is a planar, positively charged compound.

197. The pharmaceutical composition of claim 196, wherein said planar, positively charged compound is selected from the group of compounds listed in Table 11.

198. The pharmaceutical composition of claim 195, wherein said pro-heparanase binding agent is a compound having the general formula: 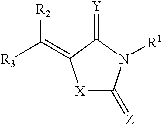
wherein:
X is O, S, NR₄ or NR₅—C(=D);
Y and Z are each independently O, S or NR₄;
R₁ is selected from the group consisting of hydrogen, a substituted or unsubstituted alkyl, a substituted or unsubstituted cycloalkyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
—(CH₂)n-CH(R₆)-Q₁(OH);
and
R₂ and R₃ are each independently selected from the group consisting of hydrogen, a substituted or unsubstituted aryl and a substituted or unsubstituted heteroaryl, at least one of R₂ and R₃ being said substituted or unsubstituted aryl or heteroaryl,
and wherein:
D is O, S or NR₄;
R₄ and R₅ are each independently selected from the group consisting of hydrogen, a substituted or unsubstituted alkyl, a substituted or unsubstituted cycloalkyl and a substituted or unsubstituted aryl;
n is integer that equals 0-20;
R₆ is selected from the group consisting of hydrogen, alkyl and Q₂(OH); and
Q₁ and Q₂ are each inependently selected from the group consisting of C═O and S(═O)₂,
whereas each of said substituted alkyl, substituted alkenyl, substituted allyl, substituted cycloalkyl, substituted aryl, substituted heteroaryl and substituted heteroalicyclic independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

199. The pharmaceutical composition of claim 198, wherein:
X is S;
Y is O; and
Z is S or O.

200. The pharmaceutical composition of claim 199, wherein Z is S.

201. The pharmaceutical composition of claim 198, wherein:
X is NR₅—C=D;
Y is O;
Z is O or S; and
D is O or S.

202. The pharmaceutical composition of claim 198, wherein R₁ is said acid-containing moiety.

203. The pharmaceutical composition of claim 202, wherein n is greater than 1.

204. The pharmaceutical composition of claim 203, wherein n equals 2-5.

205. The pharmaceutical composition of claim 198, wherein said R₁ is a substituted or unsubstituted heteroaryl.

206. The pharmaceutical composition of claim 205, wherein said heteroaryl is selected from the group consisting of terahydrothiphenyl-1,1-dioxide and 1,5-dimethyl-2-phenyl-1,2-dihydro-3-one-pyrazolyl.

207. The pharmaceutical composition of claim 198, wherein R₁ is a substituted or unsubstituted aryl.

208. The pharmaceutical composition of claim 207, wherein said aryl is selected from the group consisting of unsubstituted phenyl, 3-halophenyl, 3-trihalomethylphenyl and 3-nitrophenyl.

209. The pharmaceutical composition of claim 198, wherein at least one of R₂ and R₃ is a substituted or unsubstituted heteroaryl.

210. The pharmaceutical composition of claim 209, wherein said heteroaryl has the general formula: 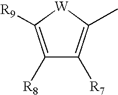
wherein:
W is O or S; and
R₇, R₈ and R₉ are each independently selected from the group consisting of hydrogen, a substituted or unsubstituted alkyl, a substituted or unsubstituted cycloalkyl, a substituted or unsubstituted aryl and a substituted or unsubstituted heteroaryl,
whereas each of said substituted alkyl, substituted cycloalkyl, substituted aryl and substituted heteroaryl independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

211. The pharmaceutical composition of claim 210, wherein:
R₇ and R₈ are each hydrogen; and
R₉ is an aryl having the general formula: 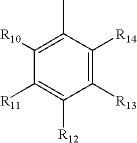
wherein each of R₁₀-R₁₄ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

212. The pharmaceutical composition of claim 211, wherein:
R₁₀ and R₁₄ are each independently selected from the group consisting of hydrogen, alkyl, cycloalkyl, hydroxy, alkoxy, thiohydroxy, thioalkoxy, aryloxy and thioaryloxy; and
R₁₁-R₁₃ are each independently selected from the group consisting of hydrogen, halo, nitro, trihaloalkyl and C-carboxy.

213. The pharmaceutical composition of claim 210, wherein:
R₇ and R₈ are each hydrogen; and
R₉ is a substituted or unsubstituted benzothiazole.

214. The pharmaceutical composition of claim 198, wherein at least one of R₂ and R₃ is a substituted or unsubstituted aryl having the general formula: 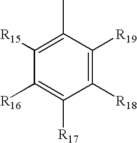
wherein each of R₁₅-R₁₉ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

215. The pharmaceutical composition of claim 211, wherein:
X is S;
Y is O;
Z is S; and
R₁ is said acid-containing moiety.

216. The pharmaceutical composition of claim 215, wherein:
n equals 2-5;
Q₁ is C═O; and
Q₂ is hydrogen.

217. The pharmaceutical composition of claim 215, wherein each of R₁₀-R₁₄ is indpenedently selected from the group consisting of hydrogen, nitro and halo.

218. The pharmaceutical composition of claim 211, wherein:
X is S;
Y is O;
Z is S; and
R₁ is selected from the group consisting of aryl, alkoxy-substituted alkyl, and heteroaryl.

219. The pharmaceutical composition of claim 218, wherein each of R₁₀-R₁₄ is indpenedently selected from the group consisting of hydrogen, nitro, C-carboxy and halo.

220. The pharmaceutical composition of claim 219, wherein at least one of R₁₀-R₁₄ is C-carboxy and said C-carboxy is a carboxylic acid group.

221. The pharmaceutical composition of claim 219, wherein R₁ is phenyl.

222. The pharmaceutical composition of claim 219, wherein R₁ is 3-methoxypropyl.

223. The pharmaceutical composition of claim 219, wherein R₁ is tetrahydrothiphenyl-1,1-dioxide.

224. The pharmaceutical composition of claim 219, wherein R₁ is 1,5-dimethyl-2-phenyl-1,2-dihydro-3-one-pyrazolyl.

225. The pharmaceutical composition of claim 176, wherein said agent capable of inhibiting heparanase heterodimerization is a peptide of no more than 50 amino acids.

226. The pharmaceutical composition of claim 225, wherein said peptide is derived from SEQ ID NO: 33 or 35.

227. The pharmaceutical composition of claim 226, wherein said peptide is as set forth in SEQ ID NO: 16, 17, 18, 19, 20, 21, 31 or 32.

228. The pharmaceutical composition of claim 174, wherein said agent capable of modulating heparanase activation is capable of increasing heparanase activation.

229. The pharmaceutical composition of claim 228, wherein said agent capable of increasing heparanase activation is:
(i) at least one protease participating in pro heparanase activation; and/or
(ii) heparin, heparin mimetic, heparan sulfate and/or heparan sulfate mimetic.

230. A method of treating a heparin binding protein-associated disease or disorder in a subject, the method comprising administering to the subject a therapeutic effective amount of an agent capable of heparin binding to the heparin binding protein, thereby treating the heparin binding protein-associated disease or disorder in the subject.

231. The method of claim 230 wherein said agent capable of inhibiting heparin binding to the heparin binding protein is a heparin binding agent.

232. The method of claim 231, wherein said heparin binding agent is a planar, positively charged compound.

233. The method of claim 232, wherein said planar, positively charged compound is selected from the group of compounds listed in Table 11.

234. The method of claim 230 wherein said agent capable of inhibiting heparin binding to the heparin binding protein is a heparin-binding protein binding agent.

235. The method of claim 234, wherein said heparin-binding protein binding agent is selected from the group of compounds which are listed in Table 15.

236. A method of identifying a protease activator of heparanase, the method comprising:
(a) providing a probe which comprises a mimetic of a cleavable site of heparanase and a cleavage reporting mechanism;
(b) subjecting said probe to a protease; and
(c) monitoring said cleavage reporting mechanism, whereby if said cleavage reporting mechanism reports of cleavage, said protease is identified as an activator of heparanase.

237. The method of claim 236, further comprising:
(d) subjecting said probe to a protease in a presence of an effective amount of an inhibitor of said protease; and
(e) assaying whether said cleavage reporting mechanism fails to report cleavage, whereby if said cleavage reporting mechanism fails to report cleavage, said protease is identified as an activator of heparanase.

238. The method of claim 236, wherein said cleavable site of heparanase is selected from the group consisting of Glu¹⁰⁹-Ser¹¹⁰ (SEQ ID NO: 1) and Gln¹⁵⁷-Lys¹⁵⁸ (SEQ ID NO: 2) in human heparanase or their equivalents in heparanases from non human, animal, origin.

239. The method of claim 236, wherein said mimetic is selected from the group consisting of Z-Pro-Lys-Lys-Glu-R (SEQ ID NO: 10) and Z-Glu-His-Tyr-Gln-R (SEQ ID NO: 11), whereby Z represents an optional first member of a FRET pair or an optional protecting group or Z is non existing and R represent a second member of a FRET pair or a self quenched fluorophore.

240. The method of claim 236, wherein said cleavage reporting mechanism comprises a quenched fluorophore.

241. The method of claim 240, wherein said quenched fluorophore is 7-amino-4-methylcoumarin (AMC).

242. A compound comprising Z-Pro-Lys-Lys-Glu-R or Z-Glu-His-Tyr-Gln-R, whereby Z represents an optional first member of a FRET pair or an optional protecting group or Z is non existing and R represent a second member of a FRET pair or a self quenched fluorophore.

243. A protease substrate mimetic comprising a peptide which comprises at least two amino acids representing a subset or all substrate residues at positions P4, P3, P2, P1, P1′, P2′, P3′, P4′ of the Glu¹⁰⁹-Ser¹¹⁰ (SEQ ID NO: 1) or the Gln¹⁵⁷-Lys¹⁵⁸ (SEQ ID NO: 2) cleavage sites of human heparanase or equivalent sites of a non-human heparanase, with the provision that P1 is represented, the protease substrate further comprising a cleavage reporting mechanism being covalently attached to said peptide, said cleavage reporting mechanism for reporting of cleavage of a bond immediately C terminally to P1.

244. The protease substrate of claim 243, wherein P4, P3, P2, P1, P1′, P2′, P3′ and P4′ are all represented.

245. The protease substrate of claim 243, wherein only P4, P3, P2 and P1 are represented.

246. The protease substrate of claim 243, wherein only P3, P2, P1, P1′, P2′, P3′ and are represented.

247. The protease substrate of claim 243, wherein said cleavage reporting mechanism comprises a Z group covalently attached at the N terminal of said peptide and an R group covalently attached at the C terminal of said peptide, whereby Z represents an optional first member of a FRET pair or an optional protecting group or Z is non existing and R represent a second member of a FRET pair or a self quenched fluorophore.

248. A method of producing active heparanase, the method comprsinig:
(a) providing a pro-heparanase;
(b) contacting said pro-heparanase with:
(i) at least one protease participating in pro-heparanase activation; and
(ii) heparin, heparin mimetic, heparan sulfate and/or heparan sulfate mimetic, thereby producing said heparanase.

249. The method of claim 248, wherein step (a) is effected by purifying said pro-heparanase from cells.

250. The method of chim 248, wherein said at least one protease participating in pro-heparanase activation is selected from the group consisting of a serine protease, a cysteine protease and an aspartic protease.

251. The method of claim 250, wherein said serine protease is elastase or cathepsin G.

252. The method of claim 250, wherein said cysteine protease is cathepsin B.

253. The method of claim 250, wherein said aspartic protease is cathepsin D.

254. A kit useful for treating a heparanase associated disease or disorder in a subject, the kit comprising a container including at least one protease participating in said pro-heparanase activation and/or heparin, heparin mimetic, heparan sulfate and/or heparan sulfate mimetic.

255. The kit of claim 254, further comprising an additional container including pro-heparanase.

256. A method of inhibiting heparanase activation comprising contacting an inactive heparanase with an agent capable of inhibiting heparanase activation, thereby inhibiting heparanase activation.

257. The method of claim 256, wherein said inactive heparanase is set forth in SEQ ID NO: 34.

258. The method of claim 256, wherein said agent capable of inhibiting heparanase activation is selected from the group consisting of:
(i) an agent capable of inhibiting at least one protease participating in said pro-heparanase activation;
(ii) an agent capable of inhibiting binding of heparin to pro-heparanase; and/or
(iii) an agent capable of inhibiting heparanase heterodimerization.

259. The method of claim 258, wherein said agent capable of inhibiting at least one protease participating in said pro-heparanase activation is selected from the group consisting of a cysteine protease inhibitor, an aspartic protease inhibitor and a serine protease inhibitor.

260. The method of claim 259, wherein said cysteine protease inhibitor is selected from the group consisitng of CA074, CA074Me, E-64, Cathepsin B inhibitor I (Z-Phe-Ala-CH₂F-A), Cathepsin B inhibitor II (Ac-Leu-Val-lysinal), Leupeptin, Leupeptin analogs, Cathepsin inhibitor I (Phe-Gly-NHO-Bz), Cathepsin inhibitor II (Phe-Gly-NHO-Bz-pMe), Cathepsin inhibitor III (Phe-Gly-NHO-Bz-pOme), Calpain inhibitor I (ALLN, N-Acetyl-Leu-Leu-NIe-CHO) and Calpain inhibitor II (ALLM, N-Acetyl-Leu-Leu-Met-CHO).

261. The method of claim 259, wherein said aspartic protease inhibitor is a cathepsin D inhibitor or a cathepsin E inhibitor each selected from the group consisting of Pepstatin A, Pepstatin A Me and a −2macroglobulin.

262. The method of claim 259, wherein said serine protease inhibitor is a compound having the general formula: 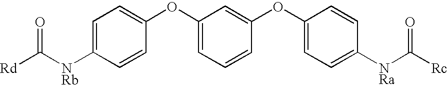
wherein:
Ra and Rb are each independently selected from the group consisting of hydrogen, alkyl, cycloalkyl or aryl; and
Rc and Rd are each independently selected from the group consisting of a substituted and unsubstituted aryl and a substituted and unsubstituted heteroaryl.

263. The method of claim 262, wherein each of Rc and Rd is a heteroaryl.

264. The method of claim 263, wherein said heteroaryl is 3-pyridine.

265. The method of claim 262, wherein each of Rc and Rd is a substituted aryl.

266. The method of claim 265, wherein said substituted aryl is a phenyl substituted by an electron withdrawing group.

267. The method of claim 258, wherein said agent capable of inhibiting at least one protease participating in said pro heparanase activation is a peptide.

268. The method of claim 267, wherein said peptide is derived from SEQ ID NO: 36.

269. The method of claim 268, wherein said peptide is as set forth in SEQ ID NO: 22, 23, 24, 25, 26, 27, 28, 29 or 30.

270. The method of claim 267, wherein said peptide is conjugated to an electrophilic group.

271. The method of claim 270, wherein said electrophilic group is derived from a chemical group selected from the group consisting of aldehydes, boronates, nitriles, β-lactams, vinyl sulfones, epoxides, halomethylketones, isocoumarin and thiodiazoles.

272. The method of claim 258, wherein said agent capable of inhibiting binding of heparin to pro heparanase, is a heparin-binding agent.

273. The method of claim 258, wherein said agent capable of inhibiting binding of heparin to pro-heparanase, is a proheparanse binding agent.

274. The method of claim 272, wherein said heparin binding agent is a planar, positively charged compound.

275. The method of claim 274, wherein said planar, positively charged compound is selected from the group of compounds listed in Table 11.

276. The method of claim 273, wherein said pro-heparanase binding agent is a compound having the general formula: 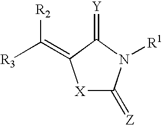
wherein:
X is O, S, NR₄ or NR₅—C(=D);
Y and Z are each independently O, S or NR₄;
R₁ is selected from the group consisting of hydrogen, a substituted or unsubstituted alkyl, a substituted or unsubstituted cycloalkyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
—(CH₂)n-CH(R₆)-Q₁(OH);
and
R₂ and R₃ are each independently selected from the group consisting of hydrogen, a substituted or unsubstituted aryl and a substituted or unsubstituted heteroaryl, at least one of R₂ and R₃ being said substituted or unsubstituted aryl or heteroaryl,
and wherein:
D is O, S or NR₄;
R₄ and R₅ are each independently selected from the group consisting of hydrogen, a substituted or unsubstituted alkyl, a substituted or unsubstituted cycloalkyl and a substituted or unsubstituted aryl;
n is integer that equals 0-20;
R₆ is selected from the group consisting of hydrogen, alkyl and Q₂(OH); and
Q₁ and Q₂ are each inependently selected from the group consisting of C═O and S(═O)₂,
whereas each of said substituted alkyl, substituted alkenyl, substituted allyl, substituted cycloalkyl, substituted aryl, substituted heteroaryl and substituted heteroalicyclic independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

277. The method of claim 276, wherein:
X is S;
Y is O; and
Z is S or O.

278. The method of claim 277, wherein Z is S.

279. The method of claim 276, wherein:
X is NR₅—C=D;
Y is O;
Z is O or S; and
D is O or S.

280. The method of claim 276, wherein R₁ is said acid-containing moiety.

281. The method of claim 280, wherein n is greater than 1.

282. The method of claim 281, wherein n equals 2-5.

283. The method of claim 276, wherein said R₁ is a substituted or unsubstituted heteroaryl.

284. The method of claim 283, wherein said heteroaryl is selected from the group consisting of terahydrothiphenyl-1,1-dioxide and 1,5-dimethyl-2-phenyl-1,2-dihydro-3-one-pyrazolyl.

285. The method of claim 276, wherein R₁ is a substituted or unsubstituted aryl.

286. The method of claim 285, wherein said aryl is selected from the group consisting of unsubstituted phenyl, 3-halophenyl, 3-trihalomethylphenyl and 3-nitrophenyl.

287. The method of claim 276, wherein at least one of R₂ and R₃ is a substituted or unsubstituted heteroaryl.

288. The method of claim 287, wherein said heteroaryl has the general formula: 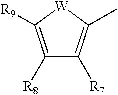
wherein:
W is O or S; and
R₇, R₈ and R₉ are each independently selected from the group consisting of hydrogen, a substituted or unsubstituted alkyl, a substituted or unsubstituted cycloalkyl, a substituted or unsubstituted aryl and a substituted or unsubstituted heteroaryl,
whereas each of said substituted alkyl, substituted cycloalkyl, substituted aryl and substituted heteroaryl independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

289. The method of claim 288, wherein:
R₇ and R₈ are each hydrogen; and
R₉ is an aryl having the general formula: 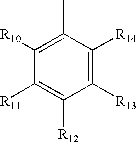
wherein each of R₁₀-R₁₄ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

290. The method of claim 289, wherein:
R₁₀ and R₁₄ are each independently selected from the group consisting of hydrogen, alkyl, cycloalkyl, hydroxy, alkoxy, thiohydroxy, thioalkoxy, aryloxy and thioaryloxy; and
R₁₁-R₁₃ are each independently selected from the group consisting of hydrogen, halo, nitro, trihaloalkyl and C-carboxy.

291. The method of claim 288, wherein:
R₇ and R₈ are each hydrogen; and
R₉ is a substituted or unsubstituted benzothiazole.

292. The method of claim 276, wherein at least one of R₂ and R₃ is a substituted or unsubstituted aryl having the general formula: 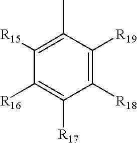
wherein each of R₁₅-R₁₉ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

293. The method of claim 289, wherein:
X is S;
Y is O;
Z is S; and
R₁ is said acid-containing moiety.

294. The method of claim 293, wherein:
n equals 2-5;
Q₁ is C═O; and
Q₂ is hydrogen.

295. The method of claim 293, wherein each of R₁₀-R₁₄ is indpenedently selected from the group consisting of hydrogen, nitro and halo.

296. The method of claim 289, wherein:
X is S;
Y is O;
Z is S; and
R₁ is selected from the group consisting of aryl, alkoxy-substituted alkyl, and heteroaryl.

297. The method of claim 296, wherein each of R₁₀-R₁₄ is indpenedently selected from the group consisting of hydrogen, nitro, C-carboxy and halo.

298. The method of claim 297, wherein at least one of R₁₀-R₁₄ is C-carboxy and said C-carboxy is a carboxylic acid group.

299. The method of claim 297, wherein R₁ is phenyl.

300. The method of claim 297, wherein R₁ is 3-methoxypropyl.

301. The method of claim 297, wherein R₁ is tetrahydrothiphenyl-1,1-dioxide.

302. The method of claim 297, wherein R₁ is 1,5-dimethyl-2-phenyl-1,2-dihydro-3-one-pyrazolyl.

303. The method of claim 258, wherein said agent capable of inhibiting heparanase heterodimerization is a peptide of no more than 50 amino acids.

304. The method of claim 303, wherein said peptide is derived from SEQ ID NO: 33 or 35.

305. The method of claim 304, wherein said peptide is as set forth in SEQ ID NO: 16, 17, 18, 19, 20, 21, 31 or 32.

306. A method of inhibiting heparanase activity, comprising contacting the heparanase with a compound having the general formula: 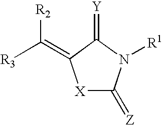
wherein:
X is O, S, NR₄ or NR₅—C(=D);
Y and Z are each independently O, S or NR₄;
R₁ is selected from the group consisting of hydrogen, a substituted or unsubstituted alkyl, a substituted or unsubstituted cycloalkyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
—(CH₂)n-CH(R₆)-Q₁(OH);
and
R₂ and R₃ are each independently selected from the group consisting of hydrogen, a substituted or unsubstituted aryl and a substituted or unsubstituted heteroaryl, at least one of R₂ and R₃ being said substituted or unsubstituted aryl or heteroaryl,
and wherein:
D is O, S or NR₄;
R₄ and R₅ are each independently selected from the group consisting of hydrogen, a substituted or unsubstituted alkyl, a substituted or unsubstituted cycloalkyl and a substituted or unsubstituted aryl;
n is integer that equals 0-20;
R₆ is selected from the group consisting of hydrogen, alkyl and Q₂(OH); and
Q₁ and Q₂ are each inependently selected from the group consisting of C═O and S(═O)₂,
whereas each of said substituted alkyl, substituted alkenyl, substituted allyl, substituted cycloalkyl, substituted aryl, substituted heteroaryl and substituted heteroalicyclic independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido,
provided that either R₁ is said acid-containing moiety or at least one of said R₂ and R₃ comprises at least one C-carboxy group.

307. The method of claim 306, wherein:
X is S;
Y is O; and
Z is S or O.

308. The method of claim 307, wherein Z is S.

309. The method of claim 306, wherein:
X is NR₅—C=D;
Y is O;
Z is O or S; and
D is O or S.

310. The method of claim 306, wherein R₁ is said acid-containing moiety.

311. The method of claim 310, wherein n is greater than 1.

312. The method of claim 311, wherein n equals 2-5.

313. The method of claim 306, wherein at least one of R₂ and R₃ is a substituted or unsubstituted heteroaryl.

314. The method of claim 313, wherein said heteroaryl has the general formula: 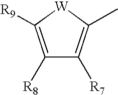
wherein:
W is O or S; and
R₇, R₈ and R₉ are each independently selected from the group consisting of hydrogen, a substituted or unsubstituted alkyl, a substituted or unsubstituted cycloalkyl, a substituted or unsubstituted aryl and a substituted or unsubstituted heteroaryl,
whereas each of said substituted alkyl, substituted cycloalkyl, substituted aryl and substituted heteroaryl independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

315. The method of claim 314, wherein:
R₇ and R₈ are each hydrogen; and
R₉ is an aryl having the general formula: 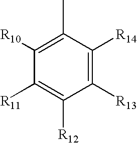
wherein each of R₁₀-R₁₄ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

316. The method of claim 315, wherein:
R₁₀ and R₁₄ are each independently selected from the group consisting of hydrogen, alkyl, cycloalkyl, hydroxy, alkoxy, thiohydroxy, thioalkoxy, aryloxy and thioaryloxy; and
R₁₁-R₁₃ are each independently selected from the group consisting of hydrogen, halo, nitro, trihaloalkyl and C-carboxy.

317. The method of claim 306, wherein at least one of R₂ and R₃ is a substituted or unsubstituted aryl having the general formula: 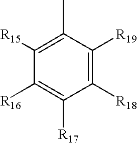
wherein each of R₁₅-R₁₉ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

318. The method of claim 315, wherein:
X is S;
Y is O;
Z is S; and
R₁ is said acid-containing moiety.

319. The method of claim 318, wherein:
n equals 2-5;
Q₁ is C═O; and
Q₂ is hydrogen.

320. The method of claim 318, wherein each of R₁₀-R₁₄ is indpenedently selected from the group consisting of hydrogen, nitro and halo.

321. The method of claim 315, wherein:
X is S;
Y is O;
Z is S; and
at least one of R₁₀-R₁₄ is C-carboxy.

322. The method of claim 321, wherein said C-carboxy is a carboxylic acid group.

323. The method of claim 321, wherein R₁ is alkyl.

324. A method of inhibiting heparanase activity, comprising contacting the heparanase with a compound having the general formula: 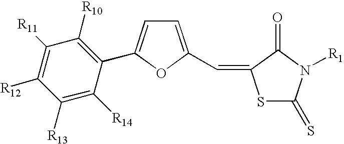
wherein:
R₁ is selected from the group consisting of hydrogen, a substituted or unsubstituted alkyl, a substituted or unsubstituted cycloalkyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
—(CH₂)n-CH(R₆)-Q₁(OH),
whereas,
n is integer that equals 0-20;
R₆ is selected from the group consisting of hydrogen, alkyl and Q₂(OH); and
Q₁ and Q₂ are each inependently selected from the group consisting of C═O and S(═O)₂; and
R₁₀-R₁₄ are each independently selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido,
provided that either R₁ is said acid-containing moiety or at least one of said R₁₀-R₁₄ is C-carboxy.

325. The method of claim 324, wherein R₁ is said acid-containing moiety.

326. The method of claim 325, wherein:
n equals 2-5;
Q₁ is C═O; and
Q₂ is hydrogen.

327. The method of claim 326, wherein each of R₁₀-R₁₄ is indpenedently selected from the group consisting of hydrogen, nitro and halo.

328. The method of claim 324, wherein at least one of said R₁₀-R₁₄ is C-carboxy.

329. The method of claim 328, wherein R₁₁ is said C-carboxy.

330. The method of claim 328, wherein said C-carboxy is a carboxylic acid group.

331. A method of treating a heparanase associated disease or disorder in a subject, the method comprising providing to the subject a therapeutic effective amount of a compound having the general formula: 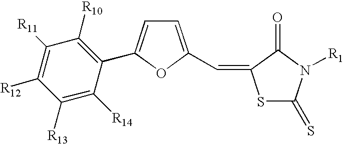
wherein:
R₁ is selected from the group consisting of hydrogen, a substituted or unsubstituted alkyl, a substituted or unsubstituted cycloalkyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
—(CH₂)n-CH(R₆)-Q₁(OH),
whereas,
n is integer that equals 0-20;
R₆ is selected from the group consisting of hydrogen, alkyl and Q₂(OH); and
Q₁ and Q₂ are each inependently selected from the group consisting of C═O and S(═O)₂; and
R₁₀-R₁₄ are each independently selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido,
provided that either R₁ is said acid-containing moiety or at least one of said R₁₀-R₁₄ is C-carboxy.

332. The method of claim 331, wherein R₁ is said acid-containing moiety.

333. The methpod of claim 332, wherein:
n equals 2-5;
Q₁ is C═O; and
Q₂ is hydrogen.

334. The method of claim 333, wherein each of R₁₀-R₁₄ is indpenedently selected from the group consisting of hydrogen, nitro and halo.

335. The method of claim 331, wherein at least one of said R₁₀-R₁₄ is C-carboxy.

336. The method of claim 335, wherein R₁₁ is said C-carboxy.

337. The method of claim 335, wherein said C-carboxy is a carboxylic acid group.

338. A method of modulating an adhesion activity of heparanase, the method comprising modulating heparin binding to heparanase, thereby modulating the adhesion activity of heparanase.

339. The method of claim 338, wherein said modulating said adhesion activity of heparanase is decreasing adhesion activity of heparanase.

340. The method of claim 339, wherein modulating heparin binding to heparanase is effected by a heparin-binding agent.

341. The method of claim 338, wherein modulating heparin binding to heparanase is effected by an agent capable of binding a heparin binding domain of heparanase.

342. The method of claim 340, wherein said heparin binding agent is a planar, positively charged compound.

343. The method of claim 342, wherein said planar, positively charged compound is selected from the group of compounds listed in Table 11.

344. The method of claim 341, wherein said agent capable of binding said heparin binding domain of heparanase is a compound having the general formula: 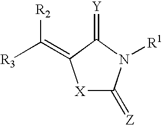
wherein:
X is O, S, NR₄ or NR₅—C(=D);
Y and Z are each independently O, S or NR₄;
R₁ is selected from the group consisting of hydrogen, a substituted or unsubstituted alkyl, a substituted or unsubstituted cycloalkyl, a substituted or unsubstituted alkenyl, a substituted or unsubstituted allyl, a substituted or unsubstituted aryl, a substituted or unsubstituted heteroaryl, a substituted or unsubstituted heteroalicyclic and an acid-containing moiety having the general formula:
—(CH₂)n-CH(R₆)-Q₁(OH);
and
R₂ and R₃ are each independently selected from the group consisting of hydrogen, a substituted or unsubstituted aryl and a substituted or unsubstituted heteroaryl, at least one of R₂ and R₃ being said substituted or unsubstituted aryl or heteroaryl,
and wherein:
D is O, S or NR₄;
R₄ and R₅ are each independently selected from the group consisting of hydrogen, a substituted or unsubstituted alkyl, a substituted or unsubstituted cycloalkyl and a substituted or unsubstituted aryl;
n is integer that equals 0-20;
R₆ is selected from the group consisting of hydrogen, alkyl and Q₂(OH); and
Q₁ and Q₂ are each inependently selected from the group consisting of C═O and S(═O)₂,
whereas each of said substituted alkyl, substituted alkenyl, substituted allyl, substituted cycloalkyl, substituted aryl, substituted heteroaryl and substituted heteroalicyclic independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

345. The method of claim 344, wherein:
X is S;
Y is O; and
Z is S or O.

346. The method of claim 345, wherein Z is S.

347. The method of claim 344, wherein:
X is NR₅—C=D;
Y is O;
Z is O or S; and
D is O or S.

348. The method of claim 344, wherein R₁ is said acid-containing moiety.

349. The method of claim 348, wherein n is greater than 1.

350. The method of claim 349, wherein n equals 2-5.

351. The method of claim 344, wherein said R₁ is a substituted or unsubstituted heteroaryl.

352. The method of claim 351, wherein said heteroaryl is selected from the group consisting of terahydrothiphenyl-1,1-dioxide and 1,5-dimethyl-2-phenyl-1,2-dihydro-3-one-pyrazolyl.

353. The method of claim 344, wherein R₁ is a substituted or unsubstituted aryl.

354. The method of claim 353, wherein said aryl is selected from the group consisting of unsubstituted phenyl, 3-halophenyl, 3-trihalomethylphenyl and 3-nitrophenyl.

355. The method of claim 344, wherein at least one of R₂ and R₃ is a substituted or unsubstituted heteroaryl.

356. The method of claim 355, wherein said heteroaryl has the general formula: 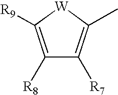
wherein:
W is O or S; and
R₇, R₈ and R₉ are each independently selected from the group consisting of hydrogen, a substituted or unsubstituted alkyl, a substituted or unsubstituted cycloalkyl, a substituted or unsubstituted aryl and a substituted or unsubstituted heteroaryl,
whereas each of said substituted alkyl, substituted cycloalkyl, substituted aryl and substituted heteroaryl independently comprises at least one substituent selected from the group consisting of halo, nitro, alkoxy, aryloxy, hydroxy, thiohydroxy, thioalkoxy, thioaryloxy, alkyl, aryl, heteroaryl, heteroalicyclic, trihaloalkyl, C-carboxy, O-carboxy, oxo, C-amido, N-amido, S-sulfonamido and N-sulfonamido.

357. The method of claim 356, wherein:
R₇ and R₈ are each hydrogen; and
R₉ is an aryl having the general formula: 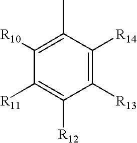
wherein each of R₁₀-R₁₄ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

358. The method of claim 357, wherein:
R₁₀ and R₁₄ are each independently selected from the group consisting of hydrogen, alkyl, cycloalkyl, hydroxy, alkoxy, thiohydroxy, thioalkoxy, aryloxy and thioaryloxy; and
R₁₁-R₁₃ are each independently selected from the group consisting of hydrogen, halo, nitro, trihaloalkyl and C-carboxy.

359. The method of claim 356, wherein:
R₇ and R₈ are each hydrogen; and
R₉ is a substituted or unsubstituted benzothiazole.

360. The method of claim 344, wherein at least one of R₂ and R₃ is a substituted or unsubstituted aryl having the general formula: 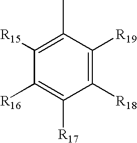
wherein each of R₁₅-R₁₉ is indpenently selected from the group consisting of hydrogen, alkyl, hydroxy, thiohydroxy, alkoxy, thioalkoxy, halo, nitro, trihaloalkyl, C-carboxy, O-carboxy, C-amido, N-amido, S-sulfonamido and N-sulfonamido, or, alternatively, at least two of R₁₀-R₁₄ form a five- or six-membered cyclic, heteroalicyclic, aromatic or heteroaromatic ring.

361. The method of claim 357, wherein:
X is S;
Y is O;
Z is S; and
R₁ is said acid-containing moiety.

362. The method of claim 361, wherein:
n equals 2-5;
Q₁ is C═O; and
Q₂ is hydrogen.

363. The method of claim 361, wherein each of R₁₀-R₁₄ is indpenedently selected from the group consisting of hydrogen, nitro and halo.

364. The method of claim 357, wherein:
X is S;
Y is O;
Z is S; and
R₁ is selected from the group consisting of aryl, alkoxy-substituted alkyl, and heteroaryl.

365. The method of claim 364, wherein each of R₁₀-R₁₄ is indpenedently selected from the group consisting of hydrogen, nitro, C-carboxy and halo.

366. The method of claim 365, wherein at least one of R₁₀-R₁₄ is C-carboxy and said C-carboxy is a carboxylic acid group.

367. The method of claim 365, wherein R₁ is phenyl.

368. The method of claim 365, wherein R₁ is 3-methoxypropyl.

369. The method of claim 365, wherein R₁ is tetrahydrothiphenyl-1,1-dioxide.

370. The method of claim 365, wherein R₁ is 1,5-dimethyl-2-phenyl-1,2-dihydro-3-one-pyrazolyl.


SEQUENCE LISTING (37 sequences)

SEQ ID NO: 1 - protein, 2 aa, Artificial sequence
  Synthetic peptide
ES

SEQ ID NO: 2 - protein, 2 aa, Artificial sequence
  Synthetic peptide
QK

SEQ ID NO: 3 - protein, 4 aa, Artificial sequence
  Synthetic peptide
PKKE

SEQ ID NO: 4 - protein, 4 aa, Artificial sequence
  Synthetic peptide
EHYQ

SEQ ID NO: 5 - protein, 535 aa, Mus musculus
MLRLLLLWLWGPLGALAQGAPAGTAPTDDVVDLEFYTKRPLRSVSPSFLSITIDASLATDPRFLTFLGSPRLRALARGLSPAYLRFGGTKTDFLIFDPDKEPTSEERSYWKSQVNHDICRSEPVSAAVLRKLQVEWPFQELLLLREQYQKEFKNSTYSRSSVDMLYSFAKCSGLDLIFGLNALLRTPDLRWNSSNAQLLLDYCSSKGYNISWELGNEPNSFWKKAHILIDGLQLGEDFVELHKLLQRSAFQNAKLYGPDIGQPRGKTVKLLRSFLKAGGEVIDSLTWHHYYLNGRIATKEDFLSSDVLDTFILSVQKILKVTKEITPGKKVWLGETSSAYGGGAPLLSNTFAAGFMWLDKLGLSAQMGIEVVMRQVFFGAGNYHLVDENFEPLPDYWLSLLFKKLVGPRVLLSRVKGPDRSKLRVYLHCTNVYHPRYQEGDLTLYVLNLHNVTKHLKVPPPLFRKPVDTYLLKPSGPDGLLSKSVQLNGQILKMVDEQTLPALTEKPLPAGSALSLPAFSYGFFVIRNAKIAACI

SEQ ID NO: 6 - protein, 545 aa, Bos taurus
MLACRKPGLRPPLLLLLPLLGPLGPCSPGTPAAAAPADDAAELEFFTERPLHLVSPAFLSFTIDANLATDPRFFTFLGSSKLRTLARGLAPAYLRFGGNKGDFLIFDPKKEPAFEERSYWLSQSNQDICKSGSIPSDVEEKLRLEWPFQEQVLLREQYQKKFTNSTYSRSSVDMLYTFASCSGLNLIFGVNALLRTTDMHWDSSNAQLLLDYCSSKNYNISWELGNEPNSFQRKAGIFINGRQLGEDFIEFRKLLGKSAFKNAKLYGPDIGQPRRNTVKMLKSFLKAGGEVIDSVTWHHYYVNGRIATKEDFLNPDILDTFISSVQKTLRIVEKIRPLKKVWLGETSSAFGGGAPFLSNTFAAGFMWLDKLGLSARMGIEVVMRQVLFGAGNYHLVDGNFEPLPDYWLSLLFKKLVGNKVLMASVKGPDRSKFRVYLHCTNTKHPRYKEGDLTLYALNLHNVTKHLELPHHLFNKQVDKYLIKPSGTDGLLSKSVQLNGQILKMVDEQTLPALTEKPLHPGSSLGMPPFSYGFFVIRNAKVAACI

SEQ ID NO: 7 - protein, 536 aa, Rattus norvegicus
MLRPLLLLWLWGRLRALTQGTPAGTAPTKDVVDLEFYTKRLFQSVSPSFLSITIDASLATDPRFLTFLGSPRLRALARGLSPAYLRFGGTKTDFLIFDPNKEPTSEERSYWQSQDNNDICGSERVSADVLRKLQMEWPFQELLLLREQYQREFKNSTYSRSSVDMLYSFAKCSRLDLIFGLNALLRTPDLRWNSSNAQLLLNYCSSKGYNISWELGNEPNSFWKKAQISIDGLQLGEDFVELHKLLQKSAFQNAKLYGPDIGQPRGKTVKLLRSFLKAGGEVIDSLTWHHYYLNGRVATKEDFLSSDVLDTFILSVQKILKVTKEMTPGKKVWLGETSSAYGGGAPLLSNTFAAGFMWLDKLGLSAQLGIEVVMRQVFFGAGNYHLVDENFEPLPDYWLSLLFKKLVGPKVLMSRVKGPDRSKLRVYLHCTNVYHPRYREGDLTLYVLNLHNVTKHLKLPPPMFSRPVDKYLLKPFGSDGLLSKSVQLNGQTLKMVDEQTLPALTEKPLPAGSSLSVPAFSYGFFVIRNAKIAACI

SEQ ID NO: 8 - protein, 543 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 9 - protein, 523 aa, Gallus gallus
MLVLLLLVLLLAVPPRRTAELQLGLREPIGAVSPAFLSLTLDASLARDPRFVALLRHPKLHTLASGLSPGFLRFGGTSTDFLIFNPNKDSTWEEKVLSEFQAKDVCEAWPSFAVVPKLLLTQWPLQEKLLLAEHSWKKHKNTTITRSTLDILHTFASSSGFRLVFGLNALLRRAGLQWDSSNAKQLLGYCAQRSYNISWELGNEPNSFRKKSGICIDGFQLGRDFVHLRQLLSQHPLYRHAELYGLDVGQPRKHTQHLLRSFMKSGGKAIDSVTWHHYYVNGRSATREDFLSPEVLDSFATAIHDVLGIVEATVPGKKVWLGETGSAYGGGAPQLSNTYVAGFMWLDKLGLAARRGIDVVMRQVSFGAGSYHLVDAGFKPLPDYWLSLLYKRLVGTRVLQASVEQADARRPRVYLHCTNPRHPKYREGDVTLFALNLSNVTQSLQLPKQLWSKSVDQYLLLPHGKDSILSREVQLNGRLLQMVDDETLPALHEMALAPGSTLGLPAFSYGFYVIRNAKAIACI

SEQ ID NO: 10 - protein, 4 aa, Artificial sequence
  Synthetic peptide
PKKE

SEQ ID NO: 11 - protein, 4 aa, Artificial sequence
  Synthetic peptide
EHYQ

SEQ ID NO: 12 - protein, 8 aa, Artificial sequence
  Synthetic peptide
PKKESTFE

SEQ ID NO: 13 - protein, 8 aa, Artificial sequence
  Synthetic peptide
EHYQKKFK

SEQ ID NO: 14 - protein, 2 aa, Artificial sequence
  Synthetic peptide
RR

SEQ ID NO: 15 - protein, 2 aa, Artificial sequence
  Synthetic peptide
FR

SEQ ID NO: 16 - protein, 22 aa, Artificial sequence
  Synthetic peptide
CPAYLRFGGTKTDFLIFDPLLE

SEQ ID NO: 17 - protein, 13 aa, Artificial sequence
  Synthetic peptide
PRFLILLGSPKLR

SEQ ID NO: 18 - protein, 13 aa, Artificial sequence
  Synthetic peptide
LRTLARGLSPAYL

SEQ ID NO: 19 - protein, 13 aa, Artificial sequence
  Synthetic peptide
FLILLGSPKLRTL

SEQ ID NO: 20 - protein, 7 aa, Artificial sequence
  Synthetic peptide
PRFLILL

SEQ ID NO: 21 - protein, 15 aa, Artificial sequence
  Synthetic peptide
CTWHHYYLNGRTATR

SEQ ID NO: 22 - protein, 10 aa, Artificial sequence
  Synthetic peptide
EQLLLREHYQ

SEQ ID NO: 23 - protein, 9 aa, Artificial sequence
  Synthetic peptide
QLLLREHYQ

SEQ ID NO: 24 - protein, 8 aa, Artificial sequence
  Synthetic peptide
LLLREHYQ

SEQ ID NO: 25 - protein, 7 aa, Artificial sequence
  Synthetic peptide
LLREHYQ

SEQ ID NO: 26 - protein, 6 aa, Artificial sequence
  Synthetic peptide
LREHYQ

SEQ ID NO: 27 - protein, 5 aa, Artificial sequence
  Synthetic peptide
REHYQ

SEQ ID NO: 28 - protein, 4 aa, Artificial sequence
  Synthetic peptide
EHYQ

SEQ ID NO: 29 - protein, 3 aa, Artificial sequence
  Synthetic peptide
HYQ

SEQ ID NO: 30 - protein, 2 aa, Artificial sequence
  Synthetic peptide
YQ

SEQ ID NO: 31 - protein, 8 aa, Artificial sequence
  Synthetic peptide
DPRFLILL

SEQ ID NO: 32 - protein, 6 aa, Artificial sequence
  Synthetic peptide
RFLILL

SEQ ID NO: 33 - protein, 386 aa, Homo sapiens
KKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 34 - protein, 508 aa, Homo sapiens
QDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 35 - protein, 74 aa, Homo sapiens
QDVVDLDFFTQEPLHLVSPSFLSVTIDANLATDPRFLILLGSPKLRTLARGLSPAYLRFGGTKTDFLIFDPKKE

SEQ ID NO: 36 - protein, 48 aa, Homo sapiens
STFEERSYWQSQVNQDICKYGSIPPDVEEKLRLEWPYQEQLLLREHYQ

SEQ ID NO: 37 - protein, 460 aa, Homo sapiens
QDVVDLDFFTQEPLHLVSPSFLSVTIDANLATDPRFLILLGSPKLRTLARGLSPAYLRFGGTKTDFLIFDPKKEKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI